SEQUENCE LISTING (118 sequences)

SEQ ID NO: 1 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 2 - DNA, 1764 bp, Kluyveromyces lactis
atggcagatcattcgagcagctcatcttcgctgcagaagaagccaattaatactatcgagcataaagacactttgggcaatgatcgggatcacaaggaagccttgaacagtgataatgataatacttctggattgaaaatcaatggtgtccccatcgaggacgctagagaggaagtgctcttaccaggttacttgtcgaagcaatattacaaattgtacggtttatgttttataacatatctgtgtgctactatgcaaggttatgatggggctttaatgggttctatctataccgaagatgcatatttgaaatactaccatttggatattaactcatcctctggtactggtctagtgttctctattttcaacgttggtcaaatttgcggtgcattctttgttcctcttatggattggaaaggtagaaaacctgctattttaattgggtgtctgggtgttgttattggtgctattatttcgtctttaacaacaacaaagagtgcattaattggtggtagatggttcgtggcctttttcgctacaatcgctaatgcagcagctccaacatactgtgcagaagtggctccagctcacttaagaggtaaggttgcaggtctttataacaccctttggtctgtcggttccattgttgctgcctttagcacttacggtaccaacaaaaacttccctaactcctccaaggcttttaagattccattatacttacaaatgatgttcccaggtcttgtgtgtatatttggttggttaatcccagaatctccaagatggttggttggtgttggccgtgaggaagaagctcgtgaattcattatcaaataccacttaaatggcgatagaactcatccattattggatatggagatggcagaaataatagaatctttccatggtacagatttatcaaaccctctagaaatgttagatgtaaggagcttattcagaacgagatcggataggtacagagcaatgttggttatacttatggcttggttcggtcaattttccggtaacaatgtgtgttcgtactatttgcctaccatgttgagaaatgttggtatgaagagtgtctcattgaatgtgttaatgaatggtgtttattccatcgtcacttggatttcttcaatttgcggtgcattctttattgataagattggtagaagggaaggtttccttggttctatctcaggtgctgcattagcattgacaggtctatctatctgtactgctcgttatgagaagactaagaagaagagtgcttccaatggtgcattggtgttcatttatctctttggtggtatcttttcttttgctttcactccaatgcaatccatgtactcaacagaagtgtctacaaacttgacgagatctaaggcccaactcctcaactttgtggtttctggtgttgcccaatttgttaatcaatttgctactccaaaggcaatgaagaatatcaaatattggttctatgtgttctacgttttcttcgatattttcgaatttattgttatctacttcttcttcgttgaaactaagggtagaagcttagaagaattagaagttgtctttgaagctccaaacccaagaaaggcatccgttgatcaagcattcttggctcaagtcagggcaactttggtccaacgaaatgacgttagagttgcaaatgctcaaaatttgaaagagcaagagcctctaaagagcgatgctgatcatgtcgaaaagctttcagaggcagaatctgtttaa

SEQ ID NO: 3 - protein, 1025 aa, Kluyveromyces lactis
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

SEQ ID NO: 4 - DNA, 3078 bp, Kluyveromyces lactis
atgtcttgccttattcctgagaatttaaggaaccccaaaaaggttcacgaaaatagattgcctactagggcttactactatgatcaggatattttcgaatctctcaatgggccttgggcttttgcgttgtttgatgcacctcttgacgctccggatgctaagaatttagactgggaaacggcaaagaaatggagcaccatttctgtgccatcccattgggaacttcaggaagactggaagtacggtaaaccaatttacacgaacgtacagtaccctatcccaatcgacatcccaaatcctcccactgtaaatcctactggtgtttatgctagaacttttgaattagattcgaaatcgattgagtcgttcgagcacagattgagatttgagggtgtggacaattgttacgagctttatgttaatggtcaatatgtgggtttcaataaggggtcccgtaacggggctgaatttgatatccaaaagtacgtttctgagggcgaaaacttagtggtcgtcaaggttttcaagtggtccgattccacttatatcgaggaccaagatcaatggtggctctctggtatttacagagacgtttctttactaaaattgcctaagaaggcccatattgaagacgttagggtcactacaacttttgtggactctcagtatcaggatgcagagctttctgtgaaagttgatgtccagggttcttcttatgatcacatcaatttcacactttacgaacctgaagatggatctaaagtttacgatgcaagctctttgttgaacgaggagaatgggaacacgactttttcaactaaagaatttatttccttctccaccaaaaagaacgaagaaacagctttcaagatcaacgtcaaggccccagaacattggaccgcagaaaatcctactttgtacaagtaccagttggatttaattggatctgatggcagtgtgattcaatctattaagcaccatgttggtttcagacaagtggagttgaaggacggtaacattactgttaatggcaaagacattctctttagaggtgtcaacagacatgatcaccatccaaggttcggtagagctgtgccattagattttgttgttagggacttgattctaatgaagaagtttaacatcaatgctgttcgtaactcgcattatccaaaccatcctaaggtgtatgacctcttcgataagctgggcttctgggtcattgacgaggcagatcttgaaactcatggtgttcaagagccatttaatcgtcatacgaacttggaggctgaatatccagatactaaaaataaactctacgatgttaatgcccattacttatcagataatccagagtacgaggtcgcgtacttagacagagcttcccaacttgtcctaagagatgtcaatcatccttcgattattatctggtccttgggtaacgaagcttgttatggcagaaaccacaaagccatgtacaagttaattaaacaattggatcctaccagacttgtgcattatgagggtgacttgaacgctttgagtgcagatatctttagtttcatgtacccaacatttgaaattatggaaaggtggaggaagaaccacactgatgaaaatggtaagtttgaaaagcctttgatcttgtgtgagtacggccatgcaatgggtaacggtcctggctctttgaaagaatatcaagagttgttctacaaggagaagttttaccaaggtggctttatctgggaatgggcaaatcacggtattgaattcgaagatgttagtactgcagatggtaagttgcataaagcttatgcttatggtggtgactttaaggaagaggttcatgacggagtgttcatcatggatggtttgtgtaacagtgagcataatcctactccgggccttgtagagtataagaaggttattgaacccgttcatattaaaattgcgcacggatctgtaacaatcacaaataagcacgacttcattacgacagaccacttattgtttatcgacaaggacacgggaaagacaatcgacgttccatctttaaagccagaagaatctgttactattccttctgatacaacttatgttgttgccgtgttgaaagatgatgctggtgttctaaaggcaggtcatgaaattgcctggggccaagctgaacttccattgaaggtacccgattttgttacagagacagcagaaaaagctgcgaagatcaacgacggtaaacgttatgtctcagttgaatccagtggattgcattttatcttggacaaattgttgggtaaaattgaaagcctaaaggtcaagggtaaggaaatttccagcaagtttgagggttcttcaatcactttctggagacctccaacgaataatgatgaacctagggactttaagaactggaagaagtacaatattgatttaatgaagcaaaacatccatggagtgagtgtcgaaaaaggttctaatggttctctagctgtagtcacggttaactctcgtatatccccagttgtattttactatgggtttgagactgttcagaagtacacgatctttgctaacaaaataaacttgaacacttctatgaagcttactggcgaatatcagcctcctgatttcccaagagttgggtacgaattctggctaggagatagttatgaatcatttgaatggttaggtcgcgggcccggcgaatcatatccggataagaaggaatctcaaagattcggtctttacgattccaaagatgtagaggaattcgtatatgactatcctcaagaaaatggaaatcatacagatacccactttttgaacatcaaatttgaaggtgcaggaaaactatcgatcttccaaaaggagaagccatttaacttcaagatttcagacgaatacggggttgatgaagctgcccacgcttgtgacgttaaaagatacggcagacactatctaaggttggaccatgcaatccatggtgttggtagcgaagcatgcggacctgctgttctggaccagtacagattgaaagctcaagatttcaactttgagtttgatctcgcttttgaataa

SEQ ID NO: 5 - DNA, 5050 bp, Artificial Sequence
  Description of Artificial Sequence Synthetic polynucleotide
gtttaaactactattagctgaattgccactgctatcgttgttagtggcgttagtgcttgcattcaaagacatggagggcgttattacgccggagctcctcgacagcagatctgatgactggtcaatatatttttgcattgaggctctgtttggaattatattttgagatgacccatctaatgtactggtatcaccagatttcatgtcgttttttaaagcggctgcttgagtcttagcaatagcgtcaccatctggtgaatcctttgaaggaaccactgacgaaggtttggacagtgacgaagaggatctttcctgctttgaattagtcgcgctgggagcagatgacgagttggtggagctgggggcaggattgctggccgtcgtgggtcctgaatgggtccttggctggtccatctctattctgaaaacggaagaggagtagggaatattactggctgaaaataagtcttgaatgaacgtatacgcgtatatttctaccaatctctcaacactgagtaatggtagttataagaaagagaccgagttagggacagttagaggcggtggagatattccttatggcatgtctggcgatgataaaacttttcaaacggcagccccgatctaaaagagctgacacccgggagttatgacaattacaacaacagaattctttctatatatgcacgaacttgtaatatggaagaaattatgacgtacaaactataaagtaaatattttacgtaacacatggtgctgttgtgcttctttttcaagagaataccaatgacgtatgactaagtttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgtgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcgacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacccttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagtcttcaccaattggtctgcagccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatggcttcagaaaaagaaattaggagagagagattcttgaacgttttccctaaattagtagaggaattgaacgcatcgcttttggcttacggtatgcctaaggaagcatgtgactggtatgcccactcattgaactacaacactccaggcggtaagctaaatagaggtttgtccgttgtggacacgtatgctattctctccaacaagaccgttgaacaattggggcaagaagaatacgaaaaggttgccattctaggttggtgcattgagttgttgcaggcttacttcttggtcgccgatgatatgatggacaagtccattaccagaagaggccaaccatgttggtacaaggttcctgaagttggggaaattgccatcaatgacgcattcatgttagaggctgctatctacaagcttttgaaatctcacttcagaaacgaaaaatactacatagatatcaccgaattgttccatgaggtcaccttccaaaccgaattgggccaattgatggacttaatcactgcacctgaagacaaagtcgacttgagtaagttctccctaaagaagcactccttcatagttactttcaagactgcttactattctttctacttgcctgtcgcattggccatgtacgttgccggtatcacggatgaaaaggatttgaaacaagccagagatgtcttgattccattgggtgaatacttccaaattcaagatgactacttagactgcttcggtaccccagaacagatcggtaagatcggtacagatatccaagataacaaatgttcttgggtaatcaacaaggcattggaacttgcttccgcagaacaaagaaagactttagacgaaaattacggtaagaaggactcagtcgcagaagccaaatgcaaaaagattttcaatgacttgaaaattgaacagctataccacgaatatgaagagtctattgccaaggatttgaaggccaaaatttctcaggtcgatgagtctcgtggcttcaaagctgatgtcttaactgcgttcttgaacaaagtttacaagagaagcaaatagaactaacgctaatcgataaaacattagatttcaaactagataaggaccatgtataagaactatatacttccaatataatatagtataagctttaagatagtatctctcgatctaccgttccacgtgactagtccaaggattttttttaacccgggatatatgtgtactttgcagttatgacgccagatggcagtagtggaagatattctttattgaaaaatagcttgtcaccttacgtacaatcttgatccggagcttttctttttttgccgattaagaattcggtcgaaaaaagaaaaggagagggccaagagggagggcattggtgactattgagcacgtgagtatacgtgattaagcacacaaaggcagcttggagtatgtctgttattaatttcacaggtagttctggtccattggtgaaagtttgcggcttgcagagcacagaggccgcagaatgtgctctagattccgatgctgacttgctgggtattatatgtgtgcccaatagaaagagaacaattgacccggttattgcaaggaaaatttcaagtcttgtaaaagcatataaaaatagttcaggcactccgaaatacttggttggcgtgtttcgtaatcaacctaaggaggatgttttggctctggtcaatgattacggcattgatatcgtccaactgcatggagatgagtcgtggcaagaataccaagagttcctcggtttgccagttattaaaagactcgtatttccaaaagactgcaacatactactcagtgcagcttcacagaaacctcattcgtttattcccttgtttgattcagaagcaggtgggacaggtgaacttttggattggaactcgatttctgactgggttggaaggcaagagagccccgaaagcttacattttatgttagctggtggactgacgccgtttaaac

SEQ ID NO: 6 - DNA, 5488 bp, Artificial Sequence
  Description of Artificial Sequence Synthetic polynucleotide
gtttaaacttgctaaattcgagtgaaacacaggaagaccagaaaatcctcatttcatccatattaacaataatttcaaatgtttatttgcattatttgaaactagggaagacaagcaacgaaacgtttttgaaaattttgagtattttcaataaatttgtagaggactcagatattgaaaaaaagctacagcaattaatacttgataagaagagtattgagaagggcaacggttcatcatctcatggatctgcacatgaacaaacaccagagtcaaacgacgttgaaattgaggctactgcgccaattgatgacaatacagacgatgataacaaaccgaagttatctgatgtagaaaaggattaaagatgctaagagatagtgatgatatttcataaataatgtaattctatatatgttaattaccttttttgcgaggcatatttatggtgaaggataagttttgaccatcaaagaaggttaatgtggctgtggtttcagggtccatacccgggagttatgacaattacaacaacagaattctttctatatatgcacgaacttgtaatatggaagaaattatgacgtacaaactataaagtaaatattttacgtaacacatggtgctgttgtgcttctttttcaagagaataccaatgacgtatgactaagtttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgtgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcgacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacccttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagtcttcaccaattggtctgcagccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgaaactctcaactaaactttgttggtgtggtattaaaggaagacttaggccgcaaaagcaacaacaattacacaatacaaacttgcaaatgactgaactaaaaaaacaaaagaccgctgaacaaaaaaccagacctcaaaatgtcggtattaaaggtatccaaatttacatcccaactcaatgtgtcaaccaatctgagctagagaaatttgatggcgtttctcaaggtaaatacacaattggtctgggccaaaccaacatgtcttttgtcaatgacagagaagatatctactcgatgtccctaactgttttgtctaagttgatcaagagttacaacatcgacaccaacaaaattggtagattagaagtcggtactgaaactctgattgacaagtccaagtctgtcaagtctgtcttgatgcaattgtttggtgaaaacactgacgtcgaaggtattgacacgcttaatgcctgttacggtggtaccaacgcgttgttcaactctttgaactggattgaatctaacgcatgggatggtagagacgccattgtagtttgcggtgatattgccatctacgataagggtgccgcaagaccaaccggtggtgccggtactgttgctatgtggatcggtcctgatgctccaattgtatttgactctgtaagagcttcttacatggaacacgcctacgatttttacaagccagatttcaccagcgaatatccttacgtcgatggtcatttttcattaacttgttacgtcaaggctcttgatcaagtttacaagagttattccaagaaggctatttctaaagggttggttagcgatcccgctggttcggatgctttgaacgttttgaaatatttcgactacaacgttttccatgttccaacctgtaaattggtcacaaaatcatacggtagattactatataacgatttcagagccaatcctcaattgttcccagaagttgacgccgaattagctactcgcgattatgacgaatctttaaccgataagaacattgaaaaaacttttgttaatgttgctaagccattccacaaagagagagttgcccaatctttgattgttccaacaaacacaggtaacatgtacaccgcatctgtttatgccgcctttgcatctctattaaactatgttggatctgacgacttacaaggcaagcgtgttggtttattttcttacggttccggtttagctgcatctctatattcttgcaaaattgttggtgacgtccaacatattatcaaggaattagatattactaacaaattagccaagagaatcaccgaaactccaaaggattacgaagctgccatcgaattgagagaaaatgcccatttgaagaagaacttcaaacctcaaggttccattgagcatttgcaaagtggtgtttactacttgaccaacatcgatgacaaatttagaagatcttacgatgttaaaaaataatcttcccccatcgattgcatcttgctgaacccccttcataaatgctttatttttttggcagcctgctttttttagctctcatttaatagagtagttttttaatctatatactaggaaaactctttatttaataacaatgatatatatatacccgggaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttgggtttaaac

SEQ ID NO: 7 - DNA, 4933 bp, Artificial Sequence
  Description of Artificial Sequence Synthetic polynucleotide
gtttaaactactcagtatattaagtttcgaattgaagggcgaactcttattcgaagtcggagtcaccacaacacttccgcccatactctccgaatcctcgtttcctaaagtaagtttacttccacttgtaggcctattattaatgatatctgaataatcctctattagggttggatcattcagtagcgcgtgcgattgaaaggagtccatgcccgacgtcgacgtgattagcgaaggcgcgtaaccattgtcatgtctagcagctatagaactaacctccttgacaccacttgcggaagtctcatcaacatgctcttccttattactcattctcttaccaagcagagaatgttatctaaaaactacgtgtatttcacctctttctcgacttgaacacgtccaactccttaagtactaccacagccaggaaagaatggatccagttctacacgatagcaaagcagaaaacacaaccagcgtacccctgtagaagcttctttgtttacagcacttgatccatgtagccatactcgaaatttcaactcatctgaaacttttcctgaaggttgaaaaagaatgccataagggtcacccgaagcttattcacgcccgggagttatgacaattacaacaacagaattctttctatatatgcacgaacttgtaatatggaagaaattatgacgtacaaactataaagtaaatattttacgtaacacatggtgctgttgtgcttctttttcaagagaataccaatgacgtatgactaagtttaggatttaatgcaggtgacggacccatctttcaaacgatttatatcagtggcgtccaaattgttaggttttgttggttcagcaggtttcctgttgtgggtcatatgactttgaaccaaatggccggctgctagggcagcacataaggataattcacctgccaagacggcacaggcaactattcttgctaattgacgtgcgttggtaccaggagcggtagcatgtgggcctcttacacctaataagtccaacatggcaccttgtggttctagaacagtaccaccaccgatggtacctacttcgatggatggcatggatacggaaattctcaaatcaccgtccacttctttcatcaatgttatacagttggaactttcgacattttgtgcaggatcttgtcctaatgccaagaaaacagctgtcactaaattagctgcatgtgcgttaaatccaccaacagacccagccattgcagatccaaccaaattcttagcaatgttcaactcaaccaatgcggaaacatcactttttaacacttttctgacaacatcaccaggaatagtagcttctgcgacgacactcttaccacgaccttcgatccagttgatggcagctggttttttgtcggtacagtagttaccagaaacggagacaacctccatatcttcccagccatactcttctaccatttgctttaatgagtattcgacacccttagaaatcatattcatacccattgcgtcaccagtagttgttctaaatctcatgaagagtaaatctcctgctagacaagtttgaatatgttgcagacgtgcaaatcttgatgtagagttaaaagcttttttaattgcgttttgtccctcttctgagtctaaccatatcttacaggcaccagatcttttcaaagttgggaaacggactactgggcctcttgtcataccatccttagttaaaacagttgttgcaccaccgccagcattgattgccttacagccacgcatggcagaagctaccaaacaaccctctgtagttgccattggtatatgataagatgtaccatcgataaccaaggggcctataacaccaacgggcaaaggcatgtaacctataacattttcacaacaagcgccaaatacgcggtcgtagtcataatttttatatggtaaacgatcagatgctaatacaggagcttctgccaaaattgaaagagccttcctacgtaccgcaaccgctctcgtagtatcacctaattttttctccaaagcgtacaaaggtaacttaccgtgaataaccaaggcagcgacctctttgttcttcaattgttttgtatttccactacttaataatgcttctaattcttctaaaggacgtattttcttatccaagctttcaatatcgcgggaatcatcttcctcactagatgatgaaggtcctgatgagctcgattgcgcagatgataaacttttgactttcgatccagaaatgactgttttattggttaaaactggtgtagaagccttttgtacaggagcagtaaaagacttcttggtgacttcagttttcaccaattggtctgcagccattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgactgccgacaacaatagtatgccccatggtgcagtatctagttacgccaaattagtgcaaaaccaaacacctgaagacattttggaagagtttcctgaaattattccattacaacaaagacctaatacccgatctagtgagacgtcaaatgacgaaagcggagaaacatgtttttctggtcatgatgaggagcaaattaagttaatgaatgaaaattgtattgttttggattgggacgataatgctattggtgccggtaccaagaaagtttgtcatttaatggaaaatattgaaaagggtttactacatcgtgcattctccgtctttattttcaatgaacaaggtgaattacttttacaacaaagagccactgaaaaaataactttccctgatctttggactaacacatgctgctctcatccactatgtattgatgacgaattaggtttgaagggtaagctagacgataagattaagggcgctattactgcggcggtgagaaaactagatcatgaattaggtattccagaagatgaaactaagacaaggggtaagtttcactttttaaacagaatccattacatggcaccaagcaatgaaccatggggtgaacatgaaattgattacatcctattttataagatcaacgctaaagaaaacttgactgtcaacccaaacgtcaatgaagttagagacttcaaatgggtttcaccaaatgatttgaaaactatgtttgctgacccaagttacaagtttacgccttggtttaagattatttgcgagaattacttattcaactggtgggagcaattagatgacctttctgaagtggaaaatgacaggcaaattcatagaatgctataacaacgcgtcaataatataggctacataaaaatcataataactttgttatcatagcaaaatgtgatataaaacgtttcatttcacctgaaaaatagtaaaaataggcgacaaaaatccttagtaatatgtaaactttattttctttatttacccgggagtcagtctgactcttgcgagagatgaggatgtaataatactaatctcgaagatgccatctaatacatatagacatacatatatatatatatacattctatatattcttacccagattctttgaggtaagacggttgggttttatcttttgcagttggtactattaagaacaatcgaatcataagcattgcttacaaagaatacacatacgaaatattaacgataatgtcaattacgaagactgaactggacggtatattgccattggtggccagaggtaaagttagagacatatatgaggtagacgctggtacgttgctgtttgttgctacggatcgtatctctgcatatgacgttattatggaaaacagcattcctgaaaaggggatcctattgaccaaactgtcagagttctggttcaagttcctgtccaacgatgttcgtaatcatttggtcgacatcgccccaggtaagactattttcgattatctacctgcaaaattgagcgaaccaaagtacaaaacgcaactagaagaccgctctctattggttcacaaacataaactaattccattggaagtaattgtcagaggctacatcaccggatctgcttggaaagagtacgtaaaaacaggtactgtgcatggtttgaaacaacctcaaggacttaaagaatctcaagagttcccagaaccaatcttcaccccatcgaccaaggctgaacaaggtgaacatgacgaaaacatctctcctgcccaggccgctgagctggtgggtgaagatttgtcacgtagagtggcagaactggctgtaaaactgtactccaagtgcaaagattatgctaaggagaagggcatcatcatcgcagacactaaattgtttaaac

SEQ ID NO: 8 - DNA, 6408 bp, Artificial Sequence
  Description of Artificial Sequence Synthetic polynucleotide
gtttaaactattgtgagggtcagttatttcatccagatataacccgagaggaaacttcttagcgtctgttttcgtaccataaggcagttcatgaggtatattttcgttattgaagcccagctcgtgaatgcttaatgctgctgaactggtgtccatgtcgcctaggtacgcaatctccacaggctgcaaaggttttgtctcaagagcaatgttattgtgcaccccgtaattggtcaacaagtttaatctgtgcttgtccaccagctctgtcgtaaccttcagttcatcgactatctgaagaaatttactaggaatagtgccatggtacagcaaccgagaatggcaatttctactcgggttcagcaacgctgcataaacgctgttggtgccgtagacatattcgaagataggattatcattcataagtttcagagcaatgtccttattctggaacttggatttatggctcttttggtttaatttcgcctgattcttgatctcctttagcttctcgacgtgggcctttttcttgccatatggatccgctgcacggtcctgttccctagcatgtacgtgagcgtatttccttttaaaccacgacgctttgtcttcattcaacgtttcccattgtttttttctactattgctttgctgtgggaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcattttttttttttccacctagcggatgactctttttttttcttagcgattggcattatcacataatgaattatacattatataaagtaatgtgatttcttcgaagaatatactaaagtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggcagaaccagcccaaaaaaagcaaaaacaaactgttcaggagcgcaaggcgtttatctcccgtatcactaatgaaactaaaattcaaatcgctatttcgctgaatggtggttatattcaaataaaagattcgattcttcctgcaaagaaggatgacgatgtagcttcccaagctactcagtcacaggtcatcgatattcacacaggtgttggctttttggatcatatgatccatgcgttggcaaaacactctggttggtctcttattgttgaatgtattggtgacctgcacattgacgatcaccatactaccgaagattgcggtatcgcattagggcaagcgttcaaagaagcaatgggtgctgtccgtggtgtaaaaagattcggtactgggttcgcaccattggatgaggcgctatcacgtgccgtagtcgatttatctagtagaccatttgctgtaatcgaccttggattgaagagagagatgattggtgatttatccactgaaatgattccacactttttggaaagtttcgcggaggcggccagaattactttgcatgttgattgtctgagaggtttcaacgatcaccacagaagtgagagtgcgttcaaggctttggctgttgccataagagaagctatttctagcaatggcaccaatgacgttccctcaaccaaaggtgttttgatgtgaagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccacccgggatggtctgcttaaatttcattctgtcttcgaaagctgaattgatactacgaaaaatttttttttgtttctctttctatctttattacataaaacttcatacacagttaagattaaaaacaactaataaataatgcctatcgcaaattagcttatgaagtccatggtaaattcgtgtttcctggcaataatagatcgtcaatttgttgctttgtggtagttttattttcaaataattggaatactagggatttgattttaagatctttattcaaattttttgcgcttaacaaacagcagccagtcccacccaagtctgtttcaaatgtctcgtaactaaaatcatcttgcaatttctttttgaaactgtcaatttgctcttgagtaatgtctcttcgtaacaaagtcaaagagcaaccgccgccaccagcaccggtaagttttgtggagccaattctcaaatcatcgctcagatttttaataagttctaatccaggatgagaaacaccgattgagacaagcagtccatgatttattcttatcaattccaatagttgttcatacagttcattattagtttctacagcctcgtcatcggtgcctttacatttacttaacttagtcatgatctctaagccttgtagggcacattcacccatggcatctagaattggcttcataacttcaggaaatttctcggtgaccaacacacgaacgcgagcaacaagatcttttgtagaccttggaattctagtataggttaggatcattggaatggctgggaaatcatctaagaacttaaaattgtttgtgtttattgttccattatgtgagtctttttcaaatagcagggcattaccataagtggccacagcgttatctattcctgaaggggtaccgtgaatacacttttcacctatgaaggcccattgattcactatatgcttatcgttttctgacagcttttccaagtcattagatcctattaacccccccaagtaggccatagctaaggccagtgatacagaaatagaggcgcttgagcccaacccagcaccgatgggtaaagtagactttaaagaaaacttaatattcttggcatgggggcataggcaaacaaacatatacaggaaacaaaacgctgcatggtagtggaaggattcggatagttgagctaacaacggatccaaaagactaacgagttcctgagacaagccatcggtggcttgttgagccttggccaatttttgggagtttacttgatcctcggtgatggcattgaaatcattgatggaccacttatgattaaagctaatgtccgggaagtccaattcaatagtatctggtgcagatgactcgcttattagcaggtaggttctcaacgcagacacactagcagcgacggcaggcttgttgtacacagcagagtgttcaccaaaaataataacctttcccggtgcagaagttaagaacggtaatgacattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgtctcagaacgtttacattgtatcgactgccagaaccccaattggttcattccagggttctctatcctccaagacagcagtggaattgggtgctgttgctttaaaaggcgccttggctaaggttccagaattggatgcatccaaggattttgacgaaattatttttggtaacgttctttctgccaatttgggccaagctccggccagacaagttgctttggctgccggtttgagtaatcatatcgttgcaagcacagttaacaaggtctgtgcatccgctatgaaggcaatcattttgggtgctcaatccatcaaatgtggtaatgctgatgttgtcgtagctggtggttgtgaatctatgactaacgcaccatactacatgccagcagcccgtgcgggtgccaaatttggccaaactgttcttgttgatggtgtcgaaagagatgggttgaacgatgcgtacgatggtctagccatgggtgtacacgcagaaaagtgtgcccgtgattgggatattactagagaacaacaagacaattttgccatcgaatcctaccaaaaatctcaaaaatctcaaaaggaaggtaaattcgacaatgaaattgtacctgttaccattaagggatttagaggtaagcctgatactcaagtcacgaaggacgaggaacctgctagattacacgttgaaaaattgagatctgcaaggactgttttccaaaaagaaaacggtactgttactgccgctaacgcttctccaatcaacgatggtgctgcagccgtcatcttggtttccgaaaaagttttgaaggaaaagaatttgaagcctttggctattatcaaaggttggggtgaggccgctcatcaaccagctgattttacatgggctccatctcttgcagttccaaaggctttgaaacatgctggcatcgaagacatcaattctgttgattactttgaattcaatgaagccttttcggttgtcggtttggtgaacactaagattttgaagctagacccatctaaggttaatgtatatggtggtgctgttgctctaggtcacccattgggttgttctggtgctagagtggttgttacactgctatccatcttacagcaagaaggaggtaagatcggtgttgccgccatttgtaatggtggtggtggtgcttcctctattgtcattgaaaagatatgattacgttctgcgattttctcatgatctttttcataaaatacataaatatataaatggctttatgtataacaggcataatttaaagttttatttgcgattcatcgtttttcaggtactcaaacgctgaggtgtgccttttgacttacttttcccgggagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacagtagcaactacaataacaacaacggcggcgtttaaac

SEQ ID NO: 9 - DNA, 6087 bp, Artificial Sequence
  Description of Artificial Sequence Synthetic polynucleotide
gtttaaacttttccaataggtggttagcaatcgtcttactttctaacttttcttaccttttacatttcagcaatatatatatatatatttcaaggatataccattctaatgtctgcccctaagaagatcgtcgttttgccaggtgaccacgttggtcaagaaatcacagccgaagccattaaggttcttaaagctatttctgatgttcgttccaatgtcaagttcgatttcgaaaatcatttaattggtggtgctgctatcgatgctacaggtgttccacttccagatgaggcgctggaagcctccaagaaggctgatgccgttttgttaggtgctgtgggtggtcctaaatggggtaccggtagtgttagacctgaacaaggtttactaaaaatccgtaaagaacttcaattgtacgccaacttaagaccatgtaactttgcatccgactctcttttagacttatctccaatcaagccacaatttgctaaaggtactgacttcgttgttgtcagagaattagtgggaggtatttactttggtaagagaaaggaagacgtttagcttgcctcgtccccgccgggtcacccggccagcgacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatggcagaaccagcccaaaaaaagcaaaaacaaactgttcaggagcgcaaggcgtttatctcccgtatcactaatgaaactaaaattcaaatcgctatttcgctgaatggtggttatattcaaataaaagattcgattcttcctgcaaagaaggatgacgatgtagcttcccaagctactcagtcacaggtcatcgatattcacacaggtgttggctttttggatcatatgatccatgcgttggcaaaacactctggttggtctcttattgttgaatgtattggtgacctgcacattgacgatcaccatactaccgaagattgcggtatcgcattagggcaagcgttcaaagaagcaatgggtgctgtccgtggtgtaaaaagattcggtactgggttcgcaccattggatgaggcgctatcacgtgccgtagtcgatttatctagtagaccatttgctgtaatcgaccttggattgaagagagagatgattggtgatttatccactgaaatgattccacactttttggaaagtttcgcggaggcggccagaattactttgcatgttgattgtctgagaggtttcaacgatcaccacagaagtgagagtgcgttcaaggctttggctgttgccataagagaagctatttctagcaatggcaccaatgacgttccctcaaccaaaggtgttttgatgtgaagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactgctgtcgattcgatactaacgccgccatccacccgggtttctcattcaagtggtaactgctgttaaaattaagatatttataaattgaagcttggtcgttccgaccaataccgtagggaaacgtaaattagctattgtaaaaaaaggaaaagaaaagaaaagaaaaatgttacatatcgaattgatcttattcctttggtagaccagtctttgcgtcaatcaaagattcgtttgtttcttgtgggcctgaaccgacttgagttaaaatcactctggcaacatccttttgcaactcaagatccaattcacgtgcagtaaagttagatgattcaaattgatggttgaaagcctcaagctgctcagtagtaaatttcttgtcccatccaggaacagagccaaacaatttatagataaatgcaaagagtttcgactcattttcagctaagtagtacaacacagcatttggacctgcatcaaacgtgtatgcaacgattgtttctccgtaaaactgattaatggtgtggcaccaactgatgatacgcttggaagtgtcattcatgtagaatattggagggaaagagtccaaacatgtggcatggaaagagttggaatccatcattgtttcctttgcaaaggtggcgaaatctttttcaacaatggctttacgcatgacttcaaatctctttggtacgacatgttcaattctttctttaaatagttcggaggttgccacggtcaattgcataccctgagtggaactcacatcctttttaatatcgctgacaactaggacacaagctttcatctgaggccagtcagagctgtctgcgatttgtactgccatggaatcatgaccatcttcagcttttcccatttcccaggccacgtatccgccaaacaacgatctacaagctgaaccagacccctttcttgctattctagatatttctgaagttgactgtggtaattggtataacttagcaattgcagagaccaatgcagcaaagccagcagcggaggaagctaaaccagctgctgtaggaaagttattttcggagacaatgtggagtttccattgagataatgtgggcaatgaggcgtccttcgattccatttcctttcttaattggcgtaggtcgcgcagacaattttgagttctttcattgtcgatgctgtgtggttctccatttaaccacaaagtgtcgcgttcaaactcaggtgcagtagccgcagaggtcaacgttctgaggtcatcttgcgataaagtcactgatatggacgaattggtgggcagattcaacttcgtgtcccttttcccccaatacttaagggttgcgatgttgacgggtgcggtaacggatgctgtgtaaacggtcattatagttttttctccttgacgttaaagtatagaggtatattaacaattttttgttgatacttttatgacatttgaataagaagtaatacaaaccgaaaatgttgaaagtattagttaaagtggttatgcagcttttgcatttatatatctgttaatagatcaaaaatcatcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataaatgtcagagttgagagccttcagtgccccagggaaagcgttactagctggtggatatttagttttagatccgaaatatgaagcatttgtagtcggattatcggcaagaatgcatgctgtagcccatccttacggttcattgcaagagtctgataagtttgaagtgcgtgtgaaaagtaaacaatttaaagatggggagtggctgtaccatataagtcctaaaactggcttcattcctgtttcgataggcggatctaagaaccctttcattgaaaaagttatcgctaacgtatttagctactttaagcctaacatggacgactactgcaatagaaacttgttcgttattgatattttctctgatgatgcctaccattctcaggaggacagcgttaccgaacatcgtggcaacagaagattgagttttcattcgcacagaattgaagaagttcccaaaacagggctgggctcctcggcaggtttagtcacagttttaactacagctttggcctccttttttgtatcggacctggaaaataatgtagacaaatatagagaagttattcataatttatcacaagttgctcattgtcaagctcagggtaaaattggaagcgggtttgatgtagcggcggcagcatatggatctatcagatatagaagattcccacccgcattaatctctaatttgccagatattggaagtgctacttacggcagtaaactggcgcatttggttaatgaagaagactggaatataacgattaaaagtaaccatttaccttcgggattaactttatggatgggcgatattaagaatggttcagaaacagtaaaactggtccagaaggtaaaaaattggtatgattcgcatatgccggaaagcttgaaaatatatacagaactcgatcatgcaaattctagatttatggatggactatctaaactagatcgcttacacgagactcatgacgattacagcgatcagatatttgagtctcttgagaggaatgactgtacctgtcaaaagtatcctgagatcacagaagttagagatgcagttgccacaattagacgttcctttagaaaaataactaaagaatctggtgccgatatcgaacctcccgtacaaactagcttattggatgattgccagaccttaaaaggagttcttacttgcttaatacctggtgctggtggttatgacgccattgcagtgattgctaagcaagatgttgatcttagggctcaaaccgctgatgacaaaagattttctaaggttcaatggctggatgtaactcaggctgactggggtgttaggaaagaaaaagatccggaaacttatcttgataaataacttaaggtagataatagtggtccatgtgacatctttataaatgtgaagtttgaagtgaccgcgcttaacatctaaccattcatcttccgatagtacttgaaattgttcctttcggcggcatgataaaattcttttaatgggtacaagctacccgggcccgggaaagattctctttttttatgatatttgtacataaactttataaatgaaattcataatagaaacgacacgaaattacaaaatggaatatgttcatagggtagacgaaactatatacgcaatctacatacatttatcaagaaggagaaaaaggaggatgtaaaggaatacaggtaagcaaattgatactaatggctcaacgtgataaggaaaaagaattgcactttaacattaatattgacaaggaggagggcaccacacaaaaagttaggtgtaacagaaaatcatgaaactatgattcctaatttatatattggaggattttctctaaaaaaaaaaaaatacaacaaataaaaaacactcaatgacctgaccatttgatggagtttaagtcaataccttcttgaaccatttcccataatggtgaaagttccctcaagaattttactctgtcagaaacggccttaacgacgtagtcgacctcctcttcagtactaaatctaccaataccaaatctgatggaagaatgggctaatgcatcatccttacccagcgcatgtaaaacataagaaggttctagggaagcagatgtacaggctgaacccgaggataatgcgatatcccttagtgccatcaataaagattctccttccacgtaggcgaaagaaacgttaacacgtttaaac

SEQ ID NO: 10 - DNA, 1737 bp, Artificial Sequence
  Description of Artificial Sequence Synthetic polynucleotide
ggatccatgtcaactttgcctatttcttctgtgtcattttcctcttctacatcaccattagtcgtggacgacaaagtctcaaccaagcccgacgttatcagacatacaatgaatttcaatgcttctatttggggagatcaattcttgacctatgatgagcctgaagatttagttatgaagaaacaattagtggaggaattaaaagaggaagttaagaaggaattgataactatcaaaggttcaaatgagcccatgcagcatgtgaaattgattgaattaattgatgctgttcaacgtttaggtatagcttaccattttgaagaagagatcgaggaagctttgcaacatatacatgttacctatggtgaacagtgggtggataaggaaaatttacagagtatttcattgtggttcaggttgttgcgtcaacagggctttaacgtctcctctggcgttttcaaagactttatggacgaaaaaggtaaattcaaagagtctttatgcaatgatgcacaaggaatattagccttatatgaagctgcatttatgagggttgaagatgaaaccatcttagacaatgctttggaattcacaaaagttcatttagatatcatagcaaaagacccatcttgcgattcttcattgcgtacacaaatccatcaagccttaaaacaacctttaagaaggagattagcaaggattgaagcattacattacatgccaatctaccaacaggaaacatctcatgatgaagtattgttgaaattagccaagttggatttcagtgttttgcagtctatgcataaaaaggaattgtcacatatctgtaagtggtggaaagatttagatttacaaaataagttaccttatgtacgtgatcgtgttgtcgaaggctacttctggatattgtccatatactatgagccacaacacgctagaacaagaatgtttttgatgaaaacatgcatgtggttagtagttttggacgatacttttgataattatggaacatacgaagaattggagatttttactcaagccgtcgagagatggtctatctcatgcttagatatgttgcccgaatatatgaaattaatctaccaagaattagtcaatttgcatgtggaaatggaagaatctttggaaaaggagggaaagacctatcagattcattacgttaaggagatggctaaagaattagttcgtaattacttagtagaagcaagatggttgaaggaaggttatatgcctactttagaagaatacatgtctgtttctatggttactggtacttatggtttgatgattgcaaggtcctatgttggcagaggagacattgttactgaagacacattcaaatgggtttctagttacccacctattattaaagcttcctgtgtaatagtaagattaatggacgatattgtatctcacaaggaagaacaagaaagaggacatgtggcttcatctatagaatgttactctaaagaatcaggtgcttctgaagaggaagcatgtgaatatattagtaggaaagttgaggatgcctggaaagtaatcaatagagaatctttgcgtccaacagccgttcccttccctttgttaatgccagcaataaacttagctagaatgtgtgaggtcttgtactctgttaatgatggttttactcatgctgagggtgacatgaaatcttatatgaagtccttcttcgttcatcctatggtcgtttgactcgag

SEQ ID NO: 11 - DNA, 7348 bp, Artificial Sequence
  Description of Artificial Sequence Synthetic polynucleotide
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgtaaggccgtttctgacagagtaaaattcttgagggaactttcaccattatgggaaatgcttcaagaaggtattgacttaaactccatcaaatggtcaggtcattgagtgttttttatttgttgtatttttttttttttagagaaaatcctccaatatcaaattaggaatcgtagtttcatgattttctgttacacctaactttttgtgtggtgccctcctccttgtcaatattaatgttaaagtgcaattctttttccttatcacgttgagccattagtatcaatttgcttacctgtattcctttactatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcaccgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggtcaaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaagattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccagccacagcacctaacaaaacggcatcaaccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattacaatggtatatccttgaaatatatataaaaaaaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctgaattggagcgacctcatgctatacctgagaaagcaacctgacctacaggaaagagttactcaagaataagaattttcgttttaaaacctaagagtcactttaaaatttgtatacacttattttttttataacttatttaataataaaaatcataaatcataagaaattcgcttatttagaagtgtcaacaacgtatctaccaacgatttgacccttttccatcttttcgtaaatttctggcaaggtagacaagccgacaaccttgattggagacttgaccaaacctctggcgaagaattgttaattaagagctcagatcttatcgtcgtcatccttgtaatccatcgatactagtgcggccgccctttagtgagggttgaattcgaattttcaaaaattcttactttttttttggatggacgcaaagaagtttaataatcatattacatggcattaccaccatatacatatccatatacatatccatatctaatcttacttatatgttgtggaaatgtaaagagccccattatcttagcctaaaaaaaccttctctttggaactttcagtaatacgcttaactgctcattgctatattgaagtacggattagaagccgccgagcgggtgacagccctccgaaggaagactctcctccgtgcgtcctcgtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatgaacgaatcaaattaacaaccataggatgataatgcgattagttttttagccttatttctggggtaattaatcagcgaagcgatgatttttgatctattaacagatatataaatgcaaaaactgcataaccactttaactaatactttcaacattttcggtttgtattacttcttattcaaatgtaataaaagtatcaacaaaaaattgttaatatacctctatactttaacgtcaaggagaaaaaaccccggatccgtaatacgactcactatagggcccgggcgtcgacatggaacagaagttgatttccgaagaagacctcgagtaagcttggtaccgcggctagctaagatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaagatccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

SEQ ID NO: 12 - DNA, 3901 bp, Artificial Sequence
  Description of Artificial Sequence Synthetic polynucleotide
gtttaaacttcaaagctcgatgcctcataaacttcggtagttatattactctgagatgacttatactctttttccaaatccacattatttggcgcaaaggtctcattggaagattccataagttggcgagagttcaatctttttgaagagccgcttaaatgtaatgatagattgtctggcattattccctcctattcttattatgcgtaggaatgtcttcgaaccgaaagatcttctctatggggtatgctttagagtgaaattaagaaaggagttttatacagatgatacctaatcatcatataagtaagagagaacagagatttaatggaaaatggaaaagggcaaattggcgctgaatcaaatagtttattatatctttacaatttgtcctgattttgtccttgtctaacttgaaaatttttcattctgatgtcatacgacttttttccggtctaggaaatcggtgaaagctttttttttttcctatcttcttgtccatcggaatttttctgtcatttcttttcctcctcgcgcttgtctactaaaatctgaattgtccaaattcagtacaaaattaatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatatcatgtcaagaataggtatccaaaacgcagcggttgaaagcatatcaagaattttgtccctgtgttttaaagtttgtggataatcgaaatctcttacattgaaaacattatcatacaatcatttattaagtagttgaagcatgtatgaactataaaagtgttactactcgttattattgcgtattttgtgatgctaaagttatgagtctcgagaagttaagattatatgaataactaaatactaaatagaaatgtaaatacagtgagaacaaaacaaaaaaaaacgaacagagaaactaaatccacattaattgagagttctatctattagaaaatgcaaactccaactaaatgggaaaacagataacctcttttatttttttttaatgtttgatattcgagtctttttcttttgttaggtttatattcatcatttcaatgaataaaagaagcttcttattttggttgcaaagaatgaaaaaaaaggattttttcatacttctaaagcttcaattataaccaaaaattttataaatgaagagaaaaaatctagtagtatcaagttaaacctattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatttttaatgttacttctcttgcagttagggaactataatgtaactcaaaataagattaaacaaactaaaataaaaagaagttatacagaaaaacccatataaaccagtactaatccataataataatacacaaaaaaactatcaaataaaaccagaaaacagattgaatagaaaaattttttcgatctccttttatattcaaaattcgatatatgaaaaagggaactctcagaaaatcaccaaatcaatttaattagatttttcttttccttctagcgttggaaagaaaaatttttctttttttttttagaaatgaaaaatttttgccgtaggaatcaccgtataaaccctgtataaacgctactctgttcacctgtgtaggctatgattgacccagtgttcattgttattgcgagagagcgggagaaaagaaccgatacaagagatccatgctggtatagttgtctgtccaacactttgatgaacttgtaggacgatgatgtgtattactagtgtcgacactgctgaagaatttgatttttctagccattcccatagacgttacaatccactaaccgattcatggatcttagtttctccacacagagctaaaagaccttggttaggtcaacaggaggctgcttacaagcccacagctccattgtatgatccaaaatgctatctatgtcctggtaacaaaagagctactggtaacctaaacccaagatatgaatcaacgtatattttccccaatgattatgctgccgttaggctcgatcaacctattttaccacagaatgattccaatgaggataatcttaaaaataggctgcttaaagtgcaatctgtgagaggcaattgtttcgtcatatgttttagccccaatcataatctaaccattccacaaatgaaacaatcagatctggttcatattgttaattcttggcaagcattgactgacgatctctccagagaagcaagagaaaatcataagcctttcaaatatgtccaaatatttgaaaacaaaggtacagccatgggttgttccaacttacatccacatggccaagcttggtgcttagaatccatccctagtgaagtttcgcaagaattgaaatcttttgataaatataaacgtgaacacaatactgatttgtttgccgattacgtcaaattagaatcaagagagaagtcaagagtcgtagtggagaatgaatcctttattgttgttgttccatactgggccatctggccatttgagaccttggtcatttcaaagaagaagcttgcctcaattagccaatttaaccaaatggtgaaggaggacctcgcctcgattttaaagcaactaactattaagtatgataatttatttgaaacgagtttcccatactcaatgggtatccatcaggctcctttgaatgcgactggtgatgaattgagtaatagttggtttcacatgcatttctacccacctttactgagatcagctactgttcggaaattcttggttggttttgaattgttaggtgagcctcgtttaaac

SEQ ID NO: 13 - DNA, 6089 bp, Artificial Sequence
  Description of Artificial Sequence Synthetic polynucleotide
gtttaaacatttcttttcctcctcgcgcttgtctactaaaatctgaattgtccaaattcagtacaaaattaatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatatcatgtcaagaataggtatccaaaacgcagcggttgaaagcatatcaagaattttgtccctgtgttttaaagtttgtggataatcgaaatctcttacattgaaaacattatcatacaatcatttattaagtagttgaagcatgtatgaactataaaagtgttactactcgttattattgcgtattttgtgatgctaaagttatgagtagaaaaaaatgagaagttgttctgaacaaagtaaaaaaaacaagtatacttactccttctttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcgccaccaaacaaagcagacggggtcagtggcactaatgattgcagctgttgctgttgccctagaaaaggcgtgactgagcgatgcgaaggtgtgcttcttggtattgtcactggagagttacgagagggtggacggttagataacagcttgactagatcactgaaacttgctcctgatttcaatggcacaggtgaaggccctactgagccaggagaaacatatttaacactgatattgttgacattttcctccggaagagtagggtattgggcgatagttgcagaaccgacaatatttttaatggcgctaccattactattgttataactgatatgcggtaatgggattgcacactgtgataacagaaacggcgcacatacctcttccagtacttgaatgtatttttcacaagtctggattttaaaagtggccagtttttttaatagcatcagaacagtgttaatttgttgtaataattgtgcggtctcgttattctcagcattcgattttgagtttgagagtagagtctttatgggtactaggactgcattgaacaagtaataagaacaattccaggcaaaatatggggtgacattatgattgtccatatagctacttacagacataacagttctttgtgctgcatcgcttaacatgatggagcatcgtttaacttcataactttgatgatcattttgatcctgttctagttgtgactttttctgggtaaaattagtgaaaaaatctcttaatacataaatgataagagacaactgtttccacttcagttcgaatcttgtaaaggatagccaagggtgttccttcaacaaattggttagagcggtggtggaaatatccatttgtaaaaactttggtgcctgtctcgaaacctcctcaatctcattacaaatcatcaagcatttttttgcacatataggacttttttctgcagttactgttttgtctagttcatagatttttgtgaaaacttgtaagagccttgctgtttcaatgatgccatgatatatggtgggacctgttgtggtacgctgcacatcgtcgacagaagaagggaaggagattgtattctgagaaagctggatggatcgaccataaagcagggacaattggatctcccaagagtagacagaccaccaaattcggcgtctttgttccagaatgctgctatcactgaaggacgaggggaggtccctattcaagcccaatgatatggccattcttatggaaaagctgtgaaaattatagctagtatttgttttctgcctccactgtgtatatcgcgacagaagatgtagggctgtcaccaaaattatggaacctgactcgaagaccttgctcgtcaaatgagatttagcattttgatagtaaaaaacatctatatcagtagattccccctctatacaccaggctccaatggctaatatgcagttaaaaaggatttgccattgatccttcgacgcgatttcaatctggttattatacaacatcattagcgtcggtgagtgcacgatagggcagtaggggtgaaaattattgagataactttgaagtaaacgggatgttgtggatctagaagccaacgtgtatctatccgtaatcatggtcgggagcctgttaacgttagagttcgtgtaattttccggtttaaagccaatagatcgaagaatacataagagagaaccgtcgccaaagaacccattattgttggggtccgttttcaggaagggcaagccatccgacatgtcatcctcttcagaccaatcaaatccatgaagagcatccctgggcataaaatccaacggaattgtggagttatcatgatgagctgccgagtcaatcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcattatagttttttctccttgacgttaaagtatagaggtatattaacaattttttgttgatacttttatgacatttgaataagaagtaatacaaactgaaaatgttgaaagtattagttaaagtggttatgcagcttttccatttatatatctgttaatagatcaaaaatcatcgcttcgctgattaattaccccagaaataaggctaaaaaactaatcgcattatcatcctatggttgttaatttgattcgttaatttgaaggtttgtggggccaggttactgccaatttttcctcttcataaccataaaagctagtattgtagaatctttattgttcggagcagtgcggcgcgaggcacatctgcgtttcaggaacgcgaccggtgaagacgaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacagtcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcttacaagcccacagctccattgtatgatccaaaatgctatctatgtcctggtaacaaaagagctactggtaacctaaacccaagatatgaatcaacgtatattttccccaatgattatgctgccgttaggctcgatcaacctattttaccacagaatgattccaatgaggataatcttaaaaataggctgcttaaagtgcaatctgtgagaggcaattgtttcgtcatatgttttagccccaatcataatctaaccattccacaaatgaaacaatcagatctggttcatattgttaattcttggcaagcattgactgacgatctctccagagaagcaagagaaaatcataagcctttcaaatatgtccaaatatttgaaaacaaaggtacagccatgggttgttccaacttacatccacatggccaagcttggtgcttagaatccatccctagtgaagtttcgcaagaattgagtttaaac

SEQ ID NO: 14 - DNA, 5812 bp, Artificial Sequence
  Description of Artificial Sequence Synthetic polynucleotide
gtttaaacatttcttttcctcctcgcgcttgtctactaaaatctgaattgtccaaattcagtacaaaattaatcagtaggacaaagggttctcgtagagtccccggaaaaaaaaaaggacaaaaagtttcaagacggcaatctctttttactgcatctcgtcagttggcaacttgccaagaacttcgcaaatgactttgacatatgataagacgtcaactgccccacgtacaataacaaaatggtagtcatatcatgtcaagaataggtatccaaaacgcagcggttgaaagcatatcaagaattttgtccctgtgttttaaagtttgtggataatcgaaatctcttacattgaaaacattatcatacaatcatttattaagtagttgaagcatgtatgaactataaaagtgttactactcgttattattgcgtattttgtgatgctaaagttatgagtagaaaaaaatgagaagttgttctgaacaaagtaaaaaaaacaagtatacttactccttctttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcgccaccaaacaaagcagacggggtcagtggcactaatgattgcagctgttgctgttgccctagaaaaggcgtgactgagcgatgcgaaggtgtgcttcttggtattgtcactggagagttacgagagggtggacggttagataacagcttgactagatcactgaaacttgctcctgatttcaatggcacaggtgaaggccctactgagccaggagaaacatatttaacactgatattgttgacattttcctccggaagagtagggtattgggcgatagttgcagaaccgacaatatttttaatggcgctaccattactattgttataactgatatgcggtaatgggattgcacactgtgataacagaaacggcgcacatacctcttccagtacttgaatgtatttttcacaagtctggattttaaaagtggccagtttttttaatagcatcagaacagtgttaatttgttgtaataattgtgcggtctcgttattctcagcattcgattttgagtttgagagtagagtctttatgggtactaggactgcattgaacaagtaataagaacaattccaggcaaaatatggggtgacattatgattgtccatatagctacttacagacataacagttctttgtgctgcatcgcttaacatgatggagcatcgtttaacttcataactttgatgatcattttgatcctgttctagttgtgactttttctgggtaaaattagtgaaaaaatctcttaatacataaatgataagagacaactgtttccacttcagttcgaatcttgtaaaggatagccaagggtgttccttcaacaaattggttagagcggtggtggaaatatccatttgtaaaaactttggtgcctgtctcgaaacctcctcaatctcattacaaatcatcaagcatttttttgcacatataggacttttttctgcagttactgttttgtctagttcatagatttttgtgaaaacttgtaagagccttgctgtttcaatgatgccatgatatatggtgggacctgttgtggtacgctgcacatcgtcgacagaagaagggaaggagattgtattctgagaaagctggatggatcgaccataaagcagggacaattggatctcccaagagtagacagaccaccaaattcggcgtctttgttccagaatgctgctatcactgaaggacgaggggaggtccctattcaagcccaatgatatggccattcttatggaaaagctgtgaaaattatagctagtatttgttttctgcctccactgtgtatatcgcgacagaagatgtagggctgtcaccaaaattatggaacctgactcgaagaccttgctcgtcaaatgagatttagcattttgatagtaaaaaacatctatatcagtagattccccctctatacaccaggctccaatggctaatatgcagttaaaaaggatttgccattgatccttcgacgcgatttcaatctggttattatacaacatcattagcgtcggtgagtgcacgatagggcagtaggggtgaaaattattgagataactttgaagtaaacgggatgttgtggatctagaagccaacgtgtatctatccgtaatcatggtcgggagcctgttaacgttagagttcgtgtaattttccggtttaaagccaatagatcgaagaatacataagagagaaccgtcgccaaagaacccattattgttggggtccgttttcaggaagggcaagccatccgacatgtcatcctcttcagaccaatcaaatccatgaagagcatccctgggcataaaatccaacggaattgtggagttatcatgatgagctgccgagtcaatcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcatctttcaggaggcttgcttctctgtcctctcttaaaatgatggcgtgcattacgtagacacaatctggagatgaagctgaaaatctggatccggaaggatgacggaaaaaatagctcataaaacagaaaaaggcccgaagtaacaataggaaaaattaattgcactaaacaaagaaaacgatattatggtgattaaactgatacagaattatgtaaatactttgaaattatagaaggtttgtagaataaaaaaaatactgggcgaatgctgtcgtcgacactagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaataggtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaacttctcttacaagcccacagctccattggtatgatccaaaatgctatctatgtcctggtaacaaaagagctactggtaacctaaacccaagatatgaatcaacgtatattttccccaatgattatgctgccgttaggctcgatcaacctattttaccacagaatgattccaatgaggataatcttaaaaataggctgcttaaagtgcaatctgtgagaggcaattgtttcgtcatatgttttagccccaatcataatctaaccattccacaaatgaaacaatcagatctggttcatattgttaattcttggcaagcattgactgacgatctctccagagaagcaagagaaaatcataagcctttcaaatatgtccaaatatttgaaaacaaaggtacagccatgggttgttccaacttacatccacatggccaagcttggtgcttagaatccatccctagtgaagtttcgcaagaattgagtttaaac

SEQ ID NO: 15 - DNA, 9217 bp, Artificial Sequence
  Description of Artificial Sequence Synthetic polynucleotide
aaacgttaattatactttattcttgttattattatactttcttagttccttttcaattgttaagaaacgatatcacaactgttacgacagagagagacccaagctagagatcacaagctaaaaaagaaccaagtttacatatatatatatatatccatattcatatttctcgagaaagagcctctatttctcattggtaagtaacttcataagagactaagttgtaaaactgtggctttgttatacggtgatttcctttggaggttgctaaggtttatggtgttgagtgcagtgtgcacgacagggaccgctagaatgcggtgagttacaaaattacacgtgacttttctggtcacgtgacctttttttctgtcagcaatccgtaggatgcgcgttggcgctacaagtgtgtcatatctgtactatatttgtacacttatatgtagttgtgacaaaagtctctgttagtactaaattaaacgatgttatatctgtggaccccctcaccttataccactacgtacatatcgttggaaaatctagatcagagggtggtaaatgaagtgtaatagtattcatttttcttataaatcatcccttccgtgatttatacaaaagaagaggagaatatgctgaatacttggtatattactctacattatactccagcccgctccgccgggtccgggagtaatacacatcatcgtcctacaagttcatcaaagtgttggacagacaactataccagcatggatctcttgtatcggttcttttctcccgctctctcgcaataacaatgaacactgggtcaatcatagcctacacaggtgaacagagtagcgtttatacagggtttatacggtgattcctacggcaaaaatttttcatttctaaaaaaaaaaagaaaaatttttctttccaacgctagaaggaaaagaaaaatctaattaaattgatttggtgattttctgagagttccctttttcatatatcgaattttgaatataaaaggagatcgaaaaaatttttctattcaatctgttttctggttttatttgatagtttttttgtgtattattattatggattagtactggtttatatgggtttttctgtataacttctttttattttagtttgtttaatcttattttgagttacattatagttccctaactgcaagagaagtaacattaaaaatgaccactcttgacgacacggcttaccggtaccgcaccagtgtcccgggggacgccgaggccatcgaggcactggatgggtccttcaccaccgacaccgtcttccgcgtcaccgccaccggggacggcttcaccctgcgggaggtgccggtggacccgcccctgaccaaggtgttccccgacgacgaatcggacgacgaatcggacgccggggaggacggcgacccggactcccggacgttcgtcgcgtacggggacgacggcgacctggcgggcttcgtggtcgtctcgtactccggctggaaccgccggctgaccgtcgaggacatcgaggtcgccccggagcaccgggggcacggggtcgggcgcgcgttgatggggctcgcgacggagttcgcccgcgagcggggcgccgggcacctctggctggaggtcaccaacgtcaacgcaccggcgatccacgcgtaccggcggatggggttcaccctctgcggcctggacaccgccctgtacgacggcaccgcctcggacggcgagcaggcgctctacatgagcatgccctgcccctgagtttaacttgatactactagattttttctcttcatttataaaatttttggttataattgaagctttagaagtatgaaaaaatccttttttttcattctttgcaaccaaaataagaagcttcttttattcattgaaatgatgaatataaacctaacaaaagaaaaagactcgaatatcaaacattaaaaaaaaataaaagaggttatctgttttcccatttagttggagtttgcattttctaatagatagaactctcaattaatgtggatttagtttctctgttcgtttttttttgttttgttctcactgtatttacatttctatttagtatttagttattcatataatcttaactgcgagcgggtggcggccaccgcggccggctcaaaggtcaatacttttcccaattcaggcaatttaaacgtacttcaatgacataccggcccatgtgctaacgtctaacagtaactgttagaataatccattaagagtctaaagcctgtggctttttaattgatgaattccacaagactttttgctgcaattaggagaagatcaagcagaataaaaaacaaattatgaagtacggaaacttcttgcacctaacaaaatatattgaaaagatggctttaaacagattctgcctctgaaagcttttcgacatgatcagcatcgctctttagaggctcttgctctttcaaattttgagcatttgcaactctaacgtcatttcgttggaccaaagttgccctgacttgagccaagaatgcttgatcaacggatgcctttcttgggtttggagcttcaaagacaacttctaattcttctaagcttctacccttagtttcaacgaagaagaagtagataacaataaattcgaaaatatcgaagaaaacgtagaacacatagaaccaatatttgatattcttcattgcctttggagtagcaaattgattaacaaattgggcaacaccagaaaccacaaagttgaggagttgggccttagatctcgtcaagtttgtagacacttctgttgagtacatggattgcattggagtgaaagcaaaagaaaagataccaccaaagagataaatgaacaccaatgcaccattggaagcactcttcttcttagtcttctcataacgagcagtacagatagatagacctgtcaatgctaatgcagcacctgagatagaaccaaggaaaccttcccttctaccaatcttatcaataaagaatgcaccgcaaattgaagaaatccaagtgacgatggaataaacaccattcattaacacattcaatgagacactcttcataccaacatttctcaacatggtaggcaaatagtacgaacacacattgttaccggaaaattgaccgaaccaagccataagtataaccaacattgctctgtacctatccgatctcgttctgaataagctccttacatctaacatttctagagggtttgataaatctgtaccatggaaagattctattatttctgccatctccatatccaataatggatgagttctatcgccatttaagtggtatttgataatgaattcacgagcttcttcctcacggccaacaccaaccaaccatcttggagattctgggattaaccaaccaaatatacacacaagacctgggaacatcatttgtaagtataatggaatcttaaaagccttggaggagttagggaagtttttgttggtaccgtaagtgctaaaggcagcaacaatggaaccgacagaccaaagggtgttataaagacctgcaaccttacctcttaagtgagctggagccacttctgcacagtatgttggagctgctgcattagcgattgtagcgaaaaaggccacgaaccatctaccaccaattaatgcactctttgttgttgttaaagacgaaataatagcaccaataacaacacccagacacccaattaaaatagcaggttttctacctttccaatccataagaggaacaaagaatgcaccgcaaatttgaccaacgttgaaaatagagaacactagaccagtaccagaggatgagttaatatccaaatggtagtatttcaaatatgcatcttcggtatagatagaacccattaaagccccatcataaccttgcatagtagcacacagatatgttataaaacataaaccgtacaatttgtaatattgcttcgacaagtaacctggtaagagcacttcctctctagcgtcctcgatggggacaccattgattttcaatccagaagtattatcattatcactgttcaaggcttccttgtgatcccgatcattgcccaaagtgtctttatgctcgatagtattaattggcttcttctgcagcgaagatgagctgctcgaatgatctgccattttcgcacgccggggccctgcaggaagtactgttttttgtgtgtgttggtgaaatatcaaaccaagttcttgatgaatttcttatttatgcaagagagagaatagaactgtactacaaatctcattgtgtgaaaatatattgtctatttatatgatttcgagactccagttttggtcattatcaccaagctcttactgctacagagaatgaacatgctcctcccccccttcttcagactatgttgttctgcacgtggataccgtcgcatgcacctaagaagcagatggtggcttgccttactgtattgtaaagatccagtctccagatctgcgaccactccgaaggttgaaacccgagcttcctgtttgctgtctcgcgccttttaaaaaaaaagcgcgattatgggccgctcgtgacagtaaaggaagcaagcagatcgaccccctgaaaatgtggtgtggttactaagcagaagcgtcttcgtcgcatatcctattcctagcgcaacaaggccccacggtgtggtttcatgtgacgtggagtcatgtaggcttgtggtgcgcacatttttactaagctcaacaaccctactggcgctgggacgcccagccgggcggcgcgccgggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctaggcccgccaccgagggcggccgcatgtcttgccttattcctgagaatttaaggaaccccaaaaaggttcacgaaaatagattgcctactagggcttactactatgatcaggatattttcgaatctctcaatgggccttgggcttttgcgttgtttgatgcacctcttgacgctccggatgctaagaatttagactgggaaacggcaaagaaatggagcaccatttctgtgccatcccattgggaacttcaggaagactggaagtacggtaaaccaatttacacgaacgtacagtaccctatcccaatcgacatcccaaatcctcccactgtaaatcctactggtgtttatgctagaacttttgaattagattcgaaatcgattgagtcgttcgagcacagattgagatttgagggtgtggacaattgttacgagctttatgttaatggtcaatatgtgggtttcaataaggggtcccgtaacggggctgaatttgatatccaaaagtacgtttctgagggcgaaaacttagtggtcgtcaaggttttcaagtggtccgattccacttatatcgaggaccaagatcaatggtggctctctggtatttacagagacgtttctttactaaaattgcctaagaaggcccatattgaagacgttagggtcactacaacttttgtggactctcagtatcaggatgcagagctttctgtgaaagttgatgtccagggttcttcttatgatcacatcaatttcacactttacgaacctgaagatggatctaaagtttacgatgcaagctctttgttgaacgaggagaatgggaacacgactttttcaactaaagaatttatttccttctccaccaaaaagaacgaagaaacagctttcaagatcaacgtcaaggccccagaacattggaccgcagaaaatcctactttgtacaagtaccagttggatttaattggatctgatggcagtgtgattcaatctattaagcaccatgttggtttcagacaagtggagttgaaggacggtaacattactgttaatggcaaagacattctctttagaggtgtcaacagacatgatcaccatccaaggttcggtagagctgtgccattagattttgttgttagggacttgattctaatgaagaagtttaacatcaatgctgttcgtaactcgcattatccaaaccatcctaaggtgtatgacctcttcgataagctgggcttctgggtcattgacgaggcagatcttgaaactcatggtgttcaagagccatttaatcgtcatacgaacttggaggctgaatatccagatactaaaaataaactctacgatgttaatgcccattacttatcagataatccagagtacgaggtcgcgtacttagacagagcttcccaacttgtcctaagagatgtcaatcatccttcgattattatctggtccttgggtaacgaagcttgttatggcagaaaccacaaagccatgtacaagttaattaaacaattggatcctaccagacttgtgcattatgagggtgacttgaacgctttgagtgcagatatctttagtttcatgtacccaacatttgaaattatggaaaggtggaggaagaaccacactgatgaaaatggtaagtttgaaaagcctttgatcttgtgtgagtacggccatgcaatgggtaacggtcctggctctttgaaagaatatcaagagttgttctacaaggagaagttttaccaaggtggctttatctgggaatgggcaaatcacggtattgaattcgaagatgttagtactgcagatggtaagttgcataaagcttatgcttatggtggtgactttaaggaagaggttcatgacggagtgttcatcatggatggtttgtgtaacagtgagcataatcctactccgggccttgtagagtataagaaggttattgaacccgttcatattaaaattgcgcacggatctgtaacaatcacaaataagcacgacttcattacgacagaccacttattgtttatcgacaaggacacgggaaagacaatcgacgttccatctttaaagccagaagaatctgttactattccttctgatacaacttatgttgttgccgtgttgaaagatgatgctggtgttctaaaggcaggtcatgaaattgcctggggccaagctgaacttccattgaaggtacccgattttgttacagagacagcagaaaaagctgcgaagatcaacgacggtaaacgttatgtctcagttgaatccagtggattgcattttatcttggacaaattgttgggtaaaattgaaagcctaaaggtcaagggtaaggaaatttccagcaagtttgagggttcttcaatcactttctggagacctccaacgaataatgatgaacctagggactttaagaactggaagaagtacaatattgatttaatgaagcaaaacatccatggagtgagtgtcgaaaaaggttctaatggttctctagctgtagtcacggttaactctcgtatatccccagttgtattttactatgggtttgagactgttcagaagtacacgatctttgctaacaaaataaacttgaacacttctatgaagcttactggcgaatatcagcctcctgatttcccaagagttgggtacgaattctggctaggagatagttatgaatcatttgaatggttaggtcgcgggcccggcgaatcatatccggataagaaggaatctcaaagattcggtctttacgattccaaagatgtagaggaattcgtatatgactatcctcaagaaaatggaaatcatacagatacccactttttgaacatcaaatttgaaggtgcaggaaaactatcgatcttccaaaaggagaagccatttaacttcaagatttcagacgaatacggggttgatgaagctgcccacgcttgtgacgttaaaagatacggcagacactatctaaggttggaccatgcaatccatggtgttggtagcgaagcatgcggacctgctgttctggaccagtacagattgaaagctcaagatttcaactttgagtttgatctcgcttttgaataagaattttatacttagataagtatgtacttacaggtatatttctatgagatactgatgtatacatgcatgataatatttaaacggttattagtgccgattgtcttgtgcgataatgacgttcctatcaaagcaatacacttaccacctattacatgggccaagaaaatattttcgaacttgtttagaatattagcacagagtatatgatgatatccgttagattatgcatgattcattcctacaactttttcgtagcataaggcgtcgggctgggagcccgcgcttggtcttttctcttcttctgtgctcttattctttgcccctgtcctaactttccatttatatagcccgtggtcgtgttctcgctgctcgtttaggcactaaacccaaaaccgataacgccttccgatgcaaagtgcagtggaaaagaaaaagggcaaagcaaataggatggtaagtcggtattgttgttgaagatgggctatgaaatgtactgagtcagagcacgccaggcagcaggttcactctgtgtaagcaaggtttgtagttcctgcggagttagagctcccagaacccaccgggacacgctcgcagggtctctagaacgggacccaggttctctgccgattccaatagccaatttggcaaagggtacacggcctccactgcattttagcaggcttcgcagcccattatgacctctaatactggtgctgggggctctgagctgcacttttccacacgccacacgtt

SEQ ID NO: 16 - DNA, 2121 bp, Artificial Sequence
  Description of Artificial Sequence Synthetic polynucleotide
gaattcgcccttntggatggcggcgttagtatcgaatcgacagcagtatagcgaccagcattcacatacgattgacgcatgatattactttctgcgcacttaacttcgcatctgggcagatgatgtcgaggcgaaaaaaaatataaatcacgctaacatttgattaaaatagaacaactacaatataaaaaaactatacaaatgacaagttcttgaaaacaagaatctttttattgtcagtactgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttgccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgaaacgtgagtcttttccttacccatggttgtttatgttcggatgtgatgtgagaactgtatcctagcaagattttaaaaggaagtatatgaaagaagaacctcagtggcaaatcctaaccttttatatttctctacaggggcgcggcgtggggacaattcaacgcgtctgtgaggggagcgtttccctgctcgcaggtctgcagcgaggagccgtaatttttgcttcgcgccgtgcggccatcaaaatgtatggatgcaaatgattatacatggggatgtatgggctaaatgtacgggcgacagtcacatcatgcccctgagctgcgcacgtcaagactgtcaaggagggtattctgggcctccatgtcgctggccgggtgacccggcggggacgaggcaagctaaacagatctgatcttgaaactgagtaagatgctcagaatacccgtcaagataagagtataatgtagagtaatataccaagtattcagcatattctcctcttcttttgtataaatcacggaagggatgatttataagaaaaatgaatactattacacttcatttaccaccctctgatctagattttccaacgatatgtacgtagtggtataaggtgagggggtccacagatataacatcgtttaatttagtactaacagagacttttgtcacaactacatataagtgtacaaatatagtacagatatgacacacttgtagcgccaacgcgcatcctacggattgctgacagaaaaaaaggtcacgtgaccagaaaagtcacgtgtaattttgtaactcaccgcattctagcggtccctgtcgtgcacactgcactcaacaccataaaccttagcaacctccaaaggaaatcaccgtataacaaagccacagttttacaacttagtctcttatgaagttacttaccaatgagaaatagaggctctttctcgagaaatatgaatatggatatatatatatatatatatatatatatatatatatgtaaacttggttcttttttagcttgtgatctctagcttgggtctctctctgtcgtaacagttgtgatatcgnaagggcgaattc

SEQ ID NO: 17 - DNA, 4510 bp, Artificial Sequence
  Description of Artificial Sequence Synthetic polynucleotide
actagtgctgaccgggataggcaatccagagcctcagtacgctggtacccgtcacaatgtagggctatatatgctggagctgctacgaaagcggcttggtctgcaggggagaacttattcccctgtgcctaatacgggcggcaaagtgcattatatagaagacgaacattgtacgatactaagatcggatggccagtacatgaatctaagtggagaacaggtgtgcaaggtctgggcccggtacgccaagtaccaagcccgacacgtagttattcatgacgagttaagtgtggcgtgtggaaaagtgcagctcagagcccccagcaccagtattagaggtcataatgggctgcgaagcctgctaaaatgcagtggaggccgtgtaccctttgccaaattggctattggaatcggcagagaacctgggtcccgttctagagaccctgcgagcgtgtcccggtgggttctgggagctctaactccgcaggaactacaaaccttgcttacacagagtgaacctgctgcctggcgtgctctgactcagtacatttcatagcccatcttcaacaacaataccgacttaccatcctatttgctttgccctttttcttttccactgcactttgcatcggaaggcgttatcggttttgggtttagtgcctaaacgagcagcgagaacacgaccacgggctatataaatggaaagttaggacaggggcaaagaataagagcacagaagaagagaaaagacgaagagcagaagcggaaaacgtatacacgtcacatatcacacacacacagagctcctcgagaagttaagattatatgaataactaaatactaaatagaaatgtaaatacagtgagaacaaaacaaaaaaaaacgaacagagaaactaaatccacattaattgagagttctatctattagaaaatgcaaactccaactaaatgggaaaacagataacctcttttatttttttttaatgtttgatattcgagtctttttcttttgttaggtttatattcatcatttcaatgaataaaagaagcttcttattttggttgcaaagaatgaaaaaaaaggattttttcatacttctaaagcttcaattataaccaaaaattttataaatgaagagaaaaaatctagtagtatcaagttaaacttaacggccttttgccagatattgattcatctcttcttccggcaccattccacctcccgtcgcccacaccagatgagtggtattacgcagttgttctgcgctgaaaccgtgcatctgttggtaacttactgatgcacacacgcgctgaggtccggccatacccgccagtgccgaaggttcaagacgaataccttcttcctgcgccagccagccaagcatgtcatacatggtttgatcgctaagggtatagaagccatccagcagacgctccattgcccgcccgacaaagcctgatgcgcgaccaactgcaaggccatccgctgcggtaaggttgtcgataccaatatcctgaacagaaatctgatcgtgtaatcctgtatggacgcctaacaacatacaaggggagtgcgttggttcggcaaaaaagcagtgaacatgatcgccaaacgccagtttaagcccgaatgcgacgccaccaggaccaccgccaacaccacacggcagatagacaaacagagggttatcagcatcgacgatacggccttgctgggcaaattgcgctttaagacgctggccagcgacggaatacccaaggaacaacgtgcgggaattttcgtcatcaataaagaaacagttcgggtcagactgcgctgctttacgtccttcctcgacggcaacaccataatcttgctcatattccacgaccgtaacgccatgcgtgcgcagtttcgcttttttccatgcccgggcatcagcagacatatgaactgtcaccttaaagccaatgcgggcgctcataatgccgattgataaccccagatttccggttgagcccacagcaatgctgtattggctaaagaactgtttaaactccggagaaagcagtttgctgtagtcatcatcaagcgtcagcaaccccgcttccagagccagtttttctgcgtgtgccaggacttcataaatcccgccgcgtgcttttatggagccggaaatgggcaaatggctatcttttttcagtaacagttgcccgctgatcggttgctgatattctttttccagccgtttttgcatagctcgaatggcaaccagttctgattcaataatccccccagtggcagcagtttcaggaaatgcttttgccagatagggtgcaaaacgggataagcgcgcatgggcgtcctgaacatcctgttcggtcaggccaacataaggtaaaccttcagccaatgaggtcgtgccaggattaaaccaggtggtttctttaagagcaaccagatcctttaccaacggatactgggcgatgagcgagttcattttagcgttttccatttttaatgttacttctcttgcagttagggaactataatgtaactcaaaataagattaaacaaactaaaataaaaagaagttatacagaaaaacccatataaaccagtactaatccataataataatacacaaaaaaactatcaaataaaaccagaaaacagattgaatagaaaaattttttcgatctccttttatattcaaaattcgatatatgaaaaagggaactctcagaaaatcaccaaatcaatttaattagatttttcttttccttctagcgttggaaagaaaaatttttctttttttttttagaaatgaaaaatttttgccgtaggaatcaccgtataaaccctgtataaacgctactctgttcacctgtgtaggctatgattgacccagtgttcattgttattgcgagagagcgggagaaaagaaccgatacaagagatccatgctggtatagttgtctgtccaacactttgatgaacttgtaggacgatgatgtgtattactagtgtcgacagtataatgtagagtaatataccaagtattcagcatattctcctcttcttttgtataaatcacggaagggatgatttataagaaaaatgaatactattacacttcatttaccaccctctgatctagattttccaacgatatgtacgtagtggtataaggtgagggggtccacagatataacatcgtttaatttagtactaacagagacttttgtcacaactacatataagtgtacaaatatagtacagatatgacacacttgtagcgccaacgcgcatcctacggattgctgacagaaaaaaaggtcacgtgaccagaaaagtcacgtgtaattttgtaactcaccgcattctagcggtccctgtcgtgcacactgcactcaacaccataaaccttagcaacctccaaaggaaatcaccgtataacaaagccacagttttacaacttagtctcttatgaagttacttaccaatgagaaatagaggctctttctcgagaaatatgaatatggatatatatatatatatatatatatatatatatatatatgtaaacttggttcttttttagcttgtgatctctagcttgggtctctctctgtcgtaacagttgtgatatcgtttcttaacaattgaaaaggaactaagaaagtataataataacaagaataaagtataattaacatgggaaagctattacaattggcattgcatccggtcgagatgaaggcagctttgaagctgaagttttgcagaacaccgctattctccatctatgatcagtccacgtctccatatctcttgcactgtttcgaactgttgaacttgacctccagatcgtttgctgctgtgatcagagagctgcatccagaattgagaaactgtgttactctcttttatttgattttaagggctttggataccatcgaagacgatatgtccatcgaacacgatttgaaaattgacttgttgcgtcacttccacgagaaattgttgttaactaaatggagtttcgacggaaatgcccccgatgtgaaggacagagccgttttgacagatttcgaatcgattcttattgaattccacaaattgaaaccagaatatcaagaagtcatcaaggagatcaccgagaaaatgggtaatggtatggccgactacatcttagatgaaaattacaacttgaatgggttgcaaaccgtccacgactacgacgtgtactgtcactacgtagctggtttggtcggtgatggtttgacccgtttgattgtcattgccaagtttgccaacgaatctttgtattctaatgagcaattgtatgaaagcatgggtcttttcctacaaaaaaccaacatcatcagagattacaatgaagatttggtcgatggtagatccttctggcccaaggaaatctggtcacaatacgctcctcagttgaaggacttcatgaaacctgaaaacgaacaactggggttggactgtataaaccacctcgtct

SEQ ID NO: 18 - DNA, 40 bp, Artificial Sequence
  Description of Artificial Sequence Synthetic primer
tgactcagtacatttcataggacagcattcgcccagtatt

SEQ ID NO: 19 - DNA, 46 bp, Artificial Sequence
  Description of Artificial Sequence Synthetic primer
agatgaagctgaaaatctggatccggaaggatgacggaaaaaatag

SEQ ID NO: 20 - DNA, 45 bp, Artificial Sequence
  Description of Artificial Sequence Synthetic primer
attttttccgtcatccttccggatccagattttcagcttcatctc

SEQ ID NO: 21 - DNA, 41 bp, Artificial Sequence
  Description of Artificial Sequence Synthetic primer
tgtgtattactagtgtcgactgagcgaagcttctgaataag

SEQ ID NO: 22 - DNA, 47 bp, Artificial Sequence
  Description of Artificial Sequence Synthetic primer
ggcgcgccgcccggctgggcgtcccagcgccagtagggttgttgagc

SEQ ID NO: 23 - DNA, 62 bp, Artificial Sequence
  Description of Artificial Sequence Synthetic primer
tttcgcacgccggggccctgcaggaagtactgttttttgtgtgtgttggtgaaatatcaaac

SEQ ID NO: 24 - DNA, 58 bp, Artificial Sequence
  Description of Artificial Sequence Synthetic primer
cgtcgggctgggagcccgcgcttggtcttttctcttcttctgtgctcttattctttgc

SEQ ID NO: 25 - DNA, 24 bp, Artificial Sequence
  Description of Artificial Sequence Synthetic primer
aacgtgtggcgtgtggaaaagtgc

SEQ ID NO: 26 - DNA, 54 bp, Artificial Sequence
  Description of Artificial Sequence Synthetic primer
aaacgttaattatactttattcttgttattattatactttcttagttccttttc

SEQ ID NO: 27 - DNA, 67 bp, Artificial Sequence
  Description of Artificial Sequence Synthetic primer
cccggacccggcggagcgggctggagtataatgtagagtaatataccaagtattcagcatattctcc

SEQ ID NO: 28 - DNA, 66 bp, Artificial Sequence
  Description of Artificial Sequence Synthetic primer
gagtgaacctgctgcctggcgtgctctgactcagtacatttcatagtggatggcggcgttagtatc

SEQ ID NO: 29 - DNA, 65 bp, Artificial Sequence
  Description of Artificial Sequence Synthetic primer
cgtgtatacgttttccgcttctgctcttcgtcttttctcttcttccgatatcacaactgttacga

SEQ ID NO: 30 - DNA, 30 bp, Artificial Sequence
  Description of Artificial Sequence Synthetic primer
gtttaaactactattagctgaattgccact

SEQ ID NO: 31 - DNA, 46 bp, Artificial Sequence
  Description of Artificial Sequence Synthetic primer
actgcaaagtacacatatatcccgggtgtcagctcttttagatcgg

SEQ ID NO: 32 - DNA, 46 bp, Artificial Sequence
  Description of Artificial Sequence Synthetic primer
ccgatctaaaagagctgacacccgggatatatgtgtactttgcagt

SEQ ID NO: 33 - DNA, 30 bp, Artificial Sequence
  Description of Artificial Sequence Synthetic primer
gtttaaacggcgtcagtccaccagctaaca

SEQ ID NO: 34 - DNA, 30 bp, Artificial Sequence
  Description of Artificial Sequence Synthetic primer
gtttaaacttgctaaattcgagtgaaacac

SEQ ID NO: 35 - DNA, 46 bp, Artificial Sequence
  Description of Artificial Sequence Synthetic primer
aaagatgaattgaaaagcttcccgggtatggaccctgaaaccacag

SEQ ID NO: 36 - DNA, 46 bp, Artificial Sequence
  Description of Artificial Sequence Synthetic primer
ctgtggtttcagggtccatacccgggaagcttttcaattcatcttt

SEQ ID NO: 37 - DNA, 30 bp, Artificial Sequence
  Description of Artificial Sequence Synthetic primer
gtttaaacccaacaataataatgtcagatc

SEQ ID NO: 38 - DNA, 30 bp, Artificial Sequence
  Description of Artificial Sequence Synthetic primer
gtttaaactactcagtatattaagtttcga

SEQ ID NO: 39 - DNA, 70 bp, Artificial Sequence
  Description of Artificial Sequence Synthetic primer
atctctcgcaagagtcagactgactcccgggcgtgaataagcttcgggtgacccttatggcattcttttt

SEQ ID NO: 40 - DNA, 70 bp, Artificial Sequence
  Description of Artificial Sequence Synthetic primer
aaaaagaatgccataagggtcacccgaagcttattcacgcccgggagtcagtctgactcttgcgagagat

SEQ ID NO: 41 - DNA, 30 bp, Artificial Sequence
  Description of Artificial Sequence Synthetic primer
gtttaaacaatttagtgtctgcgatgatga

SEQ ID NO: 42 - DNA, 30 bp, Artificial Sequence
  Description of Artificial Sequence Synthetic primer
gtttaaactattgtgagggtcagttatttc

SEQ ID NO: 43 - DNA, 44 bp, Artificial Sequence
  Description of Artificial Sequence Synthetic primer
gcggggacgaggcaagctaaactttagtatattcttcgaagaaa

SEQ ID NO: 44 - DNA, 44 bp, Artificial Sequence
  Description of Artificial Sequence Synthetic primer
tttcttcgaagaatatactaaagtttagcttgcctcgtccccgc

SEQ ID NO: 45 - DNA, 60 bp, Artificial Sequence
  Description of Artificial Sequence Synthetic primer
caatcaacgtggagggtaattctgctagcctctcccgggtggatggcggcgttagtatcg

SEQ ID NO: 46 - DNA, 60 bp, Artificial Sequence
  Description of Artificial Sequence Synthetic primer
cgatactaacgccgccatccacccgggagaggctagcagaattaccctccacgttgattg

SEQ ID NO: 47 - DNA, 30 bp, Artificial Sequence
  Description of Artificial Sequence Synthetic primer
gtttaaacgccgccgttgttgttattgtag

SEQ ID NO: 48 - DNA, 30 bp, Artificial Sequence
  Description of Artificial Sequence Synthetic primer
gtttaaacttttccaataggtggttagcaa

SEQ ID NO: 49 - DNA, 55 bp, Artificial Sequence
  Description of Artificial Sequence Synthetic primer
gggtgacccggcggggacgaggcaagctaaacgtcttcctttctcttaccaaagt

SEQ ID NO: 50 - DNA, 55 bp, Artificial Sequence
  Description of Artificial Sequence Synthetic primer
actttggtaagagaaaggaagacgtttagcttgcctcgtccccgccgggtcaccc

SEQ ID NO: 51 - DNA, 62 bp, Artificial Sequence
  Description of Artificial Sequence Synthetic primer
aatatcataaaaaaagagaatctttcccgggtggatggcggcgttagtatcgaatcgacagc

SEQ ID NO: 52 - DNA, 62 bp, Artificial Sequence
  Description of Artificial Sequence Synthetic primer
gctgtcgattcgatactaacgccgccatccacccgggaaagattctctttttttatgatatt

SEQ ID NO: 53 - DNA, 45 bp, Artificial Sequence
  Description of Artificial Sequence Synthetic primer
gtttaaacgtgttaacgtttctttcgcctacgtggaaggagaatc

SEQ ID NO: 54 - DNA, 35 bp, Artificial Sequence
  Description of Artificial Sequence Synthetic primer
tccccccgggttaaaaaaaatccttggactagtca

SEQ ID NO: 55 - DNA, 35 bp, Artificial Sequence
  Description of Artificial Sequence Synthetic primer
tccccccgggagttatgacaattacaacaacagaa

SEQ ID NO: 56 - DNA, 30 bp, Artificial Sequence
  Description of Artificial Sequence Synthetic primer
tccccccgggtatatatatatcattgttat

SEQ ID NO: 57 - DNA, 30 bp, Artificial Sequence
  Description of Artificial Sequence Synthetic primer
tccccccgggaaaagtaagtcaaaaggcac

SEQ ID NO: 58 - DNA, 30 bp, Artificial Sequence
  Description of Artificial Sequence Synthetic primer
tccccccgggatggtctgcttaaatttcat

SEQ ID NO: 59 - DNA, 45 bp, Artificial Sequence
  Description of Artificial Sequence Synthetic primer
tccccccgggtagcttgtacccattaaaagaattttatcatgccg

SEQ ID NO: 60 - DNA, 30 bp, Artificial Sequence
  Description of Artificial Sequence Synthetic primer
tccccccgggtttctcattcaagtggtaac

SEQ ID NO: 61 - DNA, 30 bp, Artificial Sequence
  Description of Artificial Sequence Synthetic primer
tccccccgggtaaataaagaaaataaagtt

SEQ ID NO: 62 - DNA, 47 bp, Artificial Sequence
  Description of Artificial Sequence Synthetic primer
aatttttgaaaattcaatataaatggcttcagaaaaagaaattagga

SEQ ID NO: 63 - DNA, 47 bp, Artificial Sequence
  Description of Artificial Sequence Synthetic primer
tcctaatttctttttctgaagccatttatattgaattttcaaaaatt

SEQ ID NO: 64 - DNA, 51 bp, Artificial Sequence
  Description of Artificial Sequence Synthetic primer
agttttcaccaattggtctgcagccattatagttttttctccttgacgtta

SEQ ID NO: 65 - DNA, 51 bp, Artificial Sequence
  Description of Artificial Sequence Synthetic primer
taacgtcaaggagaaaaaactataatggctgcagaccaattggtgaaaact

SEQ ID NO: 66 - DNA, 47 bp, Artificial Sequence
  Description of Artificial Sequence Synthetic primer
aatttttgaaaattcaatataaatgaaactctcaactaaactttgtt

SEQ ID NO: 67 - DNA, 47 bp, Artificial Sequence
  Description of Artificial Sequence Synthetic primer
aacaaagtttagttgagagtttcatttatattgaattttcaaaaatt

SEQ ID NO: 68 - DNA, 47 bp, Artificial Sequence
  Description of Artificial Sequence Synthetic primer
aatttttgaaaattcaatataaatgtctcagaacgtttacattgtat

SEQ ID NO: 69 - DNA, 47 bp, Artificial Sequence
  Description of Artificial Sequence Synthetic primer
atacaatgtaaacgttctgagacatttatattgaattttcaaaaatt

SEQ ID NO: 70 - DNA, 51 bp, Artificial Sequence
  Description of Artificial Sequence Synthetic primer
tgcagaagttaagaacggtaatgacattatagttttttctccttgacgtta

SEQ ID NO: 71 - DNA, 51 bp, Artificial Sequence
  Description of Artificial Sequence Synthetic primer
taacgtcaaggagaaaaaactataatgtcattaccgttcttaacttctgca

SEQ ID NO: 72 - DNA, 47 bp, Artificial Sequence
  Description of Artificial Sequence Synthetic primer
aatttttgaaaattcaatataaatgtcagagttgagagccttcagtg

SEQ ID NO: 73 - DNA, 47 bp, Artificial Sequence
  Description of Artificial Sequence Synthetic primer
cactgaaggctctcaactctgacatttatattgaattttcaaaaatt

SEQ ID NO: 74 - DNA, 51 bp, Artificial Sequence
  Description of Artificial Sequence Synthetic primer
ggtaacggatgctgtgtaaacggtcattatagttttttctccttgacgtta

SEQ ID NO: 75 - DNA, 51 bp, Artificial Sequence
  Description of Artificial Sequence Synthetic primer
taacgtcaaggagaaaaaactataatgaccgtttacacagcatccgttacc

SEQ ID NO: 76 - DNA, 47 bp, Artificial Sequence
  Description of Artificial Sequence Synthetic primer
aatttttgaaaattcaatataaatgactgccgacaacaatagtatgc

SEQ ID NO: 77 - DNA, 47 bp, Artificial Sequence
  Description of Artificial Sequence Synthetic primer
gcatactattgttgtcggcagtcatttatattgaattttcaaaaatt

SEQ ID NO: 78 - DNA, 70 bp, Artificial Sequence
  Description of Artificial Sequence Synthetic primer
ggtaagacggttgggttttatcttttgcagttggtactattaagaacaatcacaggaaacagctatgacc

SEQ ID NO: 79 - DNA, 70 bp, Artificial Sequence
  Description of Artificial Sequence Synthetic primer
ttgcgttttgtactttggttcgctcaattttgcaggtagataatcgaaaagttgtaaaacgacggccagt

SEQ ID NO: 80 - DNA, 44 bp, Artificial Sequence
  Description of Artificial Sequence Synthetic primer
ttgtgatgctaaagttatgagtctcgagaagttaagattatatg

SEQ ID NO: 81 - DNA, 44 bp, Artificial Sequence
  Description of Artificial Sequence Synthetic primer
catataatcttaacttctcgagactcataactttagcatcacaa

SEQ ID NO: 82 - DNA, 28 bp, Artificial Sequence
  Description of Artificial Sequence Synthetic primer
gtttaaacttcaaagctcgatgcctcat

SEQ ID NO: 83 - DNA, 28 bp, Artificial Sequence
  Description of Artificial Sequence Synthetic primer
gtttaaacgaggctcacctaacaattca

SEQ ID NO: 84 - DNA, 44 bp, Artificial Sequence
  Description of Artificial Sequence Synthetic primer
gatgtgtattactagtgtcgacactgctgaagaatttgattttt

SEQ ID NO: 85 - DNA, 44 bp, Artificial Sequence
  Description of Artificial Sequence Synthetic primer
aaaaatcaaattcttcagcagtgtcgacactagtaatacacatc

SEQ ID NO: 86 - DNA, 28 bp, Artificial Sequence
  Description of Artificial Sequence Synthetic primer
gtttaaactcaattcttgcgaaacttca

SEQ ID NO: 87 - DNA, 44 bp, Artificial Sequence
  Description of Artificial Sequence Synthetic primer
attcatataatcttaacttctcttacaagcccacagctccattg

SEQ ID NO: 88 - DNA, 44 bp, Artificial Sequence
  Description of Artificial Sequence Synthetic primer
caatggagctgtgggcttgtaagagaagttaagattatatgaat

SEQ ID NO: 89 - DNA, 44 bp, Artificial Sequence
  Description of Artificial Sequence Synthetic primer
tggggctctttacatttccacagtcgacactagtaatacacatc

SEQ ID NO: 90 - DNA, 44 bp, Artificial Sequence
  Description of Artificial Sequence Synthetic primer
gatgtgtattactagtgtcgactgtggaaatgtaaagagcccca

SEQ ID NO: 91 - DNA, 44 bp, Artificial Sequence
  Description of Artificial Sequence Synthetic primer
cgatagaagacagtagcttcattatagttttttctccttgacgt

SEQ ID NO: 92 - DNA, 44 bp, Artificial Sequence
  Description of Artificial Sequence Synthetic primer
acgtcaaggagaaaaaactataatgaagctactgtcttctatcg

SEQ ID NO: 93 - DNA, 63 bp, Artificial Sequence
  Description of Artificial Sequence Synthetic primer
gttctgaacaaagtaaaaaaaacaagtatacttactccttctttgggtttggtggggtatctt

SEQ ID NO: 94 - DNA, 20 bp, Artificial Sequence
  Description of Artificial Sequence Synthetic primer
actagtgctgaccgggatag

SEQ ID NO: 95 - DNA, 44 bp, Artificial Sequence
  Description of Artificial Sequence Synthetic primer
atcttaacttctcgaggagctctgtgtgtgtgtgatatgtgacg

SEQ ID NO: 96 - DNA, 44 bp, Artificial Sequence
  Description of Artificial Sequence Synthetic primer
cgtcacatatcacacacacacagagctcctcgagaagttaagat

SEQ ID NO: 97 - DNA, 44 bp, Artificial Sequence
  Description of Artificial Sequence Synthetic primer
gtatattactctacattatactgtcgacactagtaatacacatc

SEQ ID NO: 98 - DNA, 44 bp, Artificial Sequence
  Description of Artificial Sequence Synthetic primer
gatgtgtattactagtgtcgacagtataatgtagagtaatatac

SEQ ID NO: 99 - DNA, 44 bp, Artificial Sequence
  Description of Artificial Sequence Synthetic primer
ccaattgtaatagctttcccatgttaattatactttattcttgt

SEQ ID NO: 100 - DNA, 44 bp, Artificial Sequence
  Description of Artificial Sequence Synthetic primer
acaagaataaagtataattaacatgggaaagctattacaattgg

SEQ ID NO: 101 - DNA, 20 bp, Artificial Sequence
  Description of Artificial Sequence Synthetic primer
agacgaggtggtttatacag

SEQ ID NO: 102 - DNA, 28 bp, Artificial Sequence
  Description of Artificial Sequence Synthetic primer
gtttaaactcaattcttgcgaaacttca

SEQ ID NO: 103 - DNA, 63 bp, Artificial Sequence; identical to SEQ ID NO: 93
  Description of Artificial Sequence Synthetic primer

SEQ ID NO: 104 - DNA, 63 bp, Artificial Sequence
  Description of Artificial Sequence Synthetic primer
aagataccccaccaaacccaaagaaggagtaagtatacttgttttttttactttgttcagaac

SEQ ID NO: 105 - DNA, 28 bp, Artificial Sequence
  Description of Artificial Sequence Synthetic primer
gtttaaacatttcttttcctcctcgcgc

SEQ ID NO: 106 - DNA, 44 bp, Artificial Sequence
  Description of Artificial Sequence Synthetic primer
aaaatactgggcgaatgctgtcgtcgacactagtaatacacatc

SEQ ID NO: 107 - DNA, 44 bp, Artificial Sequence
  Description of Artificial Sequence Synthetic primer
gatgtgtattactagtgtcgacgacagcattcgcccagtatttt

SEQ ID NO: 108 - DNA, 32 bp, Artificial Sequence
  Description of Artificial Sequence Synthetic primer
taataaggatccatgtcaactttgcctatttc

SEQ ID NO: 109 - DNA, 32 bp, Artificial Sequence
  Description of Artificial Sequence Synthetic primer
ttatagctagctcaaacgaccataggatgaac

SEQ ID NO: 110 - DNA, 47 bp, Artificial Sequence
  Description of Artificial Sequence Synthetic primer
cctgcagggccccggcgtgcgaaaatggcagatcattcgagcagctc

SEQ ID NO: 111 - DNA, 50 bp, Artificial Sequence
  Description of Artificial Sequence Synthetic primer
gcgagcgggtggcggccaccgcggccggctcaaaggtcaatacttttccc

SEQ ID NO: 112 - DNA, 58 bp, Artificial Sequence
  Description of Artificial Sequence Synthetic primer
aggcccgccaccgagggcggccgcatgtcttgccttattcctgagaatttaaggaacc

SEQ ID NO: 113 - DNA, 60 bp, Artificial Sequence
  Description of Artificial Sequence Synthetic primer
caagcgcgggctcccagcccgacgccttatgctacgaaaaagttgtaggaatgaatcatg

SEQ ID NO: 114 - DNA, 60 bp, Artificial Sequence
  Description of Artificial Sequence Synthetic primer
ccagcccgctccgccgggtccgggagtaatacacatcatcgtcctacaagttcatcaaag

SEQ ID NO: 115 - DNA, 71 bp, Artificial Sequence
  Description of Artificial Sequence Synthetic primer
ccgcggtggccgccacccgctcgcagttaagattatatgaataactaaatactaaatagaaatgtaaatac

SEQ ID NO: 116 - DNA, 48 bp, Artificial Sequence
  Description of Artificial Sequence Synthetic primer
ggacgcccagccgggcggcgcgccgggccagaaaaaggaagtgtttcc

SEQ ID NO: 117 - DNA, 69 bp, Artificial Sequence
  Description of Artificial Sequence Synthetic primer
gcggccgccctcggtggcgggcctagataattacttccttgatgatctgtaaaaaagagaaaaagaaag

SEQ ID NO: 118 - DNA, 17 bp, Artificial Sequence
  Description of Artificial Sequence Synthetic oligonucleotide
cgsnnnnnnnnnnnscg

(19) United States
(12) Patent Application Publication
(10) Pub. No.: US 2009/0253174 A1
(43) Pub. Date: Oct. 8, 2009

(54) Expression of Heterologous Sequences

(76) Inventors: Zach Serber, Sausalito, CA (US); Arthur Leo Kruckerberg, Wilmington, DE (US)

Correspondence Address: WILSON SONSINI GOODRICH & ROSATI, 650 PAGE MILL ROAD, PALO ALTO, CA, 94304-1050, US

(21) Appl. No.: 12/419,803

(22) Filed: Apr. 7, 2009

Related U.S. Application Data
(60) Provisional application No. 61/123,562, filed on Apr. 8, 2008.

(51) Int. Cl. C12P 21/06; C12N 1/15

(52) U.S. Cl. 435/69.1; 435/254.11

(57) ABSTRACT

The present invention provides compositions and methods for expression of heterologous sequences. The compositions and methods are particularly useful for expressing large quantity of heterologous proteins and nucleic acids of therapeutic, diagnostic and industrial applications.

### CROSS-REFERENCE

This application claims the benefit of U.S. Provisional Application No. 61/123,562 filed Apr. 8, 2008, which application is incorporated herein by reference in its entirety.

### BACKGROUND OF THE INVENTION

Numerous human therapeutics, vaccines, diagnostics, as well as many industrial agents and commercially valuable products can be produced recombinantly utilizing a wide range of expression systems. Gene expression systems are broadly categorized into two classes: inducible and non-inducible (constitutive) systems. Inducible gene expression systems typically have minimal protein production, for example negligible or almost no protein production, being produced until an inducing agent is provided. On the other hand, non-inducible (constitutive) gene expression systems typically does not need such induction, and protein production generally occurs continuously from a constitute gene expression system.

In some situations, such as certain research settings, inducible gene expression systems are more desirable because it permits control of protein production at physiologically optimal time points and levels (e.g., levels that are not toxic to the physiological state of the cell).

A frequently used inducible gene expression system is based on the GAL regulon in yeast. Yeast can utilize galactose as a carbon source and use the GAL genes to import galactose and metabolize it inside the cell. The GAL genes include structural genes GAL1, GAL2, GAL7, and GAL10 genes, which respectively encode galactokinase, galactose permease, α-D-galactose-1-phosphate uridyltransferase, and uridine diphosphogalactose-4-epimerase, and regulator genes GAL4, GAL80, and GAL3. The GAL4 and GAL80 gene products or proteins are respectively positive and negative regulators of the expression of the GALE, GAL2, GAL7, and GAL10 genes.

In the absence of galactose, very little expression of the structural proteins (Gal1p, Gal2p, Gal7p, and Gal10p) is typically detected. Gal4p activates transcription by binding upstream activating sequences (UAS), such as those of the GAL structural genes. However, Gal4p transcription activity is inhibited by Gal80p. In the absence of galactose, Gal80p interacts with Gal4p, preventing Gal4p transcriptional activity. In the presence of galactose, however, Gal3p interacts with Gal80p, relieving Gal4p repression by Gal80p. This allows expression of genes downstream of Gal4p binding sequences, such as the GAL1, GAL2, GAL7, and GAL10.

The conventional galactose-inducible expression system has a number of profound drawbacks even though it provides tight regulation and supports high level of production of heterologous proteins. The most severe limitation is that it requires direct supplementation of galactose to activate expression of the heterologous protein. In practice, a large quantity of galactose is directly added to the culture medium to induce expression of a given sequence after the host cell reaches a desired density. However, galactose is an expensive commodity. In many instances, it is cost prohibitive to utilize galactose for large-scale production, especially of products with low profit margin. Thus, there remains a considerable need for an alternative design of an expression system that is equally robust but more cost effective than the conventional system. The present invention satisfies this need and provides related advantages as well.

### SUMMARY OF THE INVENTION

The present invention provides methods for the heterologous production of products in cell culture using a galactose-inducible expression system.

In one aspect, the present invention encompasses a method of expressing a heterologous sequence in a host cell, comprising: culturing the host cell in a medium and under conditions such that the heterologous sequence is expressed, wherein the heterologous sequence is operably linked to a galactose-inducible regulatory element, and expression of the heterologous sequence is induced without directly supplementing galactose to said medium. In some embodiments, the medium comprises a non-galactose sugar (e.g., lactose) and expression of said heterologous sequence is induced by the non-galactose sugar and to a level comparable to that obtained by culturing said host cell in a galactose-supplemented medium, wherein quantities of the supplemented galactose and non-galactose sugar are comparable as measured in moles. The heterologous sequence whose expression can be induced includes any nucleic acid sequences such as antisense molecules, siRNA, miRNA, EGS, aptamers, and ribozymes. The nucleic acid sequences can also encode proteinaceous products. Where designed, the heterologous sequences can be present on a single expression vector or on multiple expression vectors.

The present invention also provides a method of producing an isoprenoid in a host cell comprising: culturing a host cell expressing one or more heterologous sequences encoding one or more enzymes in a mevalonate-independent deoxyxylulose 5-phosphate (DXP) pathway or mevalonate (MEV) pathway, wherein said one or more heterologous sequences are operably linked to a galactose-inducible regulatory element and expression of said one or more heterologous sequences is induced without directly supplementing galactose to said medium. In some embodiments, expression of the one or more heterologous sequences is induced in the presence of lactose. The heterologous sequences can be present on a single expression vector or on multiple expression vectors. The isoprenoid produced may be combustible. In some embodiments, the host cell further comprises an exogenous sequence encoding a prenyltransferase or an isoprenoid synthase. In some embodiments, the methods comprise medium comprising lactose and/or lactase.

In yet another aspect of the present invention is the host cell used in methods of the present invention. The host cell can comprise a galactose transporter, such as GAL2 galactose transporter. In other embodiments, the host cell can comprise a lactose transporter. The host cell may also comprise an exogenous sequence encoding a lactase enzyme. In some embodiments, the exogenous sequence encodes a secretable lactase.

In some embodiments, the host cell can produce an isoprenoid via deoxyxylulose 5-phosphate (DXP) pathway, wherein the heterologous sequence encodes one or more enzymes in the mevalonate-independent deoxyxylulose 5-phosphate (DXP) pathway of mevalonate (MEV) pathway, wherein the heterologous sequence encodes one or more enzymes in the pathway. In some embodiments, the isoprenoid produced is combustible. In some embodiments, the galactose-inducible regulatory element is episomal. In other embodiments, the galactose-inducible regulatory element is integrated into the genome of said host cell. The galactose-inducible regulatory element may comprise a galactose-inducible promoter selected from the group consisting of a GAL7, GAL2, GAL1 GAL10, GAL3, GCY1, GAL80 promoter. The host cell may also comprise a lactase or biologically active fragment thereof. The host cell may exhibit a reduced capability to catabolize galactose. In some embodiments, the host cell lacks a functional GAL1, GAL7, and/or GAL10 protein. In some embodiments, the host cell expresses Gal4 protein. In some embodiments, the host cell expresses GAL4 under the control of a constitutive promoter.

In yet another aspect, the host cell is a prokaryotic cell. In other embodiments, the host cell is a eukaryotic cell, such as a Saccharomyces cerevisiae cell. The host cell can be modified to express a heterologous sequence operably linked to a galactose-inducible regulatory element when cultured in a medium, wherein expression of said heterologous sequence is induced without directly supplementing galactose to said medium. The medium may comprise a non-galactose compound, for example, lactose, and expression of the heterologous sequence is induced to a level comparable to that obtained by culturing the host cell in a medium supplemented with moles of galactose comparable to the non-galactose compound. Further provided in the present invention is a cell culture comprising the subject host cells.

The present invention also provides an expression vector. The subject expression vector typically comprises a first heterologous sequence operably linked to a galactose-inducible regulatory element and a second heterologous sequence encoding a lactase or biologically active fragment thereof, wherein upon introduction to a host cell, said expression vector causes expression of said first heterologous sequence in said host cell when said cell is cultured in a medium that is supplemented with lactose in an amount sufficient to induce expression of said first heterologous sequence. The second heterologous sequence may encode a lactase or biologically active fragment that hydrolyzes lactose to glucose and galactose. The expression vector can further comprise a heterologous sequence encoding an enzyme or biologically active fragment thereof of the DXP pathway or the MEV pathway. The vector can also comprise a heterologous sequence encoding a lactose transporter or galactose transporter.

Also provided herein is a set of expression vectors comprising at least a first expression vector and at least a second expression vector, wherein the first expression vector comprises a first heterologous sequence operably linked to a galactose-inducible regulatory element, and a second expression vector comprise a second heterologous sequence encoding a lactase or biologically active fragment thereof wherein upon introduction to a host cell, the set of expression vectors cause expression of the first heterologous sequence in the host cell when the cell is cultured in a medium, wherein the medium is supplemented with lactose in an amount sufficient to induce expression of the first heterologous sequence. The second heterologous sequence encoding a lactase or biologically active fragment thereof can be expressed to hydrolyze lactose to glucose and galactose. The set of expression vectors can further comprise a heterologous sequence encoding an enzyme or biologically active fragment thereof of the DXP pathway or the MEV pathway. The set can also further comprise a heterologous sequence encoding a lactose transporter of a galactose transporter. Also provided is a kit comprising an expression vector of the present invention or the set of expression vectors and instructions for use of the corresponding kit.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic representation of the conversion of lactose into β-D-galactose and D-glucose as catalyzed by lactase.

FIG. 2 shows maps of DNA fragments ERG20-P_{GAL}-tHMGR (A), ERG13-P_{GAL}-tHMGR (B), IDI1-P_{GAL}-tHMGR (C), ERG10-P_{GAL}-ERG12 (D), and ERG8-P_{GAL}-ERG19 (E).

FIG. 3 shows a map of plasmid pAM404.

FIG. 4 shows maps of DNA fragments GAL7^{4 to 1021}HPH-GAL1^{1637 to 2587} (A), GAL7^{125 to 598}-pH-GAL1^{4 to 549}-GAL4-GAL1^{1585 to 2088} (B), and GAL7^{126 to 598}-HPH-P_{GAL4OC}-GAL4-GAL1^{1585 to 2088} (C).

FIG. 5 shows a map of DNA fragment 5′ locus-NatR-LAC12-P_{TDH1}-P_{PGK1}-LAC4-3′ locus.

FIG. 6 shows production of γ-farnesene by host strains Y435 and Y596 in culture medium comprising galactose or lactose.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### General Techniques:

The practice of the present invention employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### DEFINITIONS

Unless defined otherwise, all technical and scientific terms used herein have the same meaning as commonly understood by one of ordinary skill in the art to which this invention belongs. Reference is made here to a number of terms that shall be defined to have the following meanings:

The term “consteuct” or “vector” refers to a recombinant nucleic acid, generally recombinant DNA, that has been generated for the purpose of the expression and/or propagation of a specific nucleotide sequence(s), or is to be used in the construction of other recombinant nucleotide sequences.

The term “exogenous” refers to what is not normally found in and/or produced by a given cell in nature.

The term “endogenous” refers to what is normally found in and/or produced by a given cell in nature.

The term “galactose-inducible expression system” refers to the combination of a galactose induction machinery and a galactose-inducible regulatory element.

The term “galactose induction machinery” refers to the collection of proteins that induces transcription of a heterologous sequence operably linked a galactose-inducible regulatory element in the presence of galactose. An example of a galactose induction machinery is the collection of yeast proteins Gal3p, Gal4p, and Gal80p, or functional homologs thereof.

The term “galactose-inducible expression cassette” refers to a nucleotide sequence that comprises a heterologous sequence operably linked to a galactose-inducible regulatory element. The galactose-inducible expression cassette is induced (i.e., its heterologous sequence is transcribed into mRNA) when galactose is present.

The term “galactose-inducible promoter” refers to a promoter sequence that is bound by regulated by a transcriptional activator regulated by galactose. For example, the galactose-inducible promoter is regulated by Gal4p or functional homologs thereof.

The term “heterologous” refers to what is not normally found in nature. The term “heterologous production of protein” refers to the production of a protein by a cell that does not normally produce the protein, or to the production of a protein at a level at which it is not normally produced by a cell. The term “heterologous sequence” refers to a nucleotide sequence that is not normally found in a given cell in nature. The term encompasses a nucleic acid wherein at least one of the following is true: (a) the nucleic acid that is exogenously introduced into a given cell (hence “exogenous sequence” even though the sequence can be foreign or native to the recipient cell); (b) the nucleic acid comprises a nucleotide sequence that is naturally found in a given cell (e.g., the nucleic acid comprises a nucleotide sequence that is endogenous to the cell) but the nucleic acid is either produced in an unnatural (e.g., greater than expected or greater than naturally found) amount in the cell, or the nucleotide sequence differs from the endogenous nucleotide sequence such that the same encoded protein (having the same or substantially the same amino acid sequence) as found endogenously is produced in an unnatural (e.g., greater than expected or greater than naturally found) amount in the cell; (c) the nucleic acid comprises two or more nucleotide sequences or segments that are not found in the same relationship to each other in nature (e.g., the nucleic acid is recombinant).

The term “host cell” refers to any cell that comprises a galactose induction machinery, and includes any suitable archae, bacterial, or eukaryotic cell.

The terms “induce”, “induction”, and “inducible” refer to the activation of transcription or relief of repression of transcription of a nucleotide sequence. The term “galactose-inducible” refers to the activation of transcription or relief of repression of transcription of a nucleotide sequence in the presence of galactose.

The term “expression” refers to the process by which a polynucleotide is transcribed into mRNA and/or the process by which the transcribed mRNA (also referred to as “transcript”) is subsequently being translated into peptides, polypeptides, or proteins. The transcripts and the encoded polypeptides are collectedly referred to as “gene product.” If the polynucleotide is derived from genomic DNA, expression may include splicing of the mRNA in a eukaryotic cell.

Operably linked” or “operatively linked” refers to a juxtaposition wherein the components so described are in a relationship permitting them to function in their intended manner. For instance, a promoter sequence is operably linked to a coding sequence if the promoter sequence promotes transcription of the coding sequence.

The term “isoprenoid” refers to a molecule derivable from isopentenyl diphosphate (“IPP”), and it may comprise one or more IPP unites.

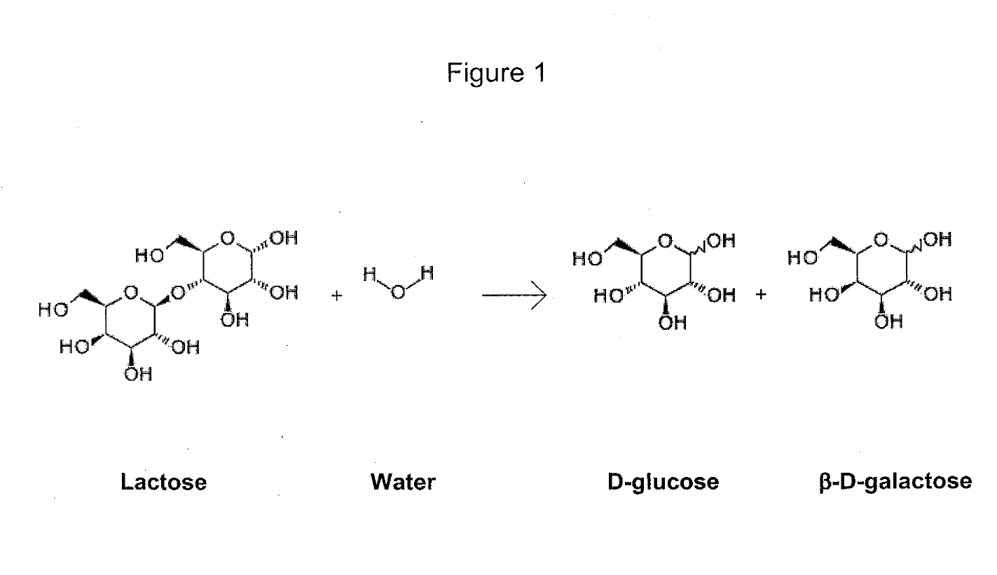

The term “lactase” refers to an enzyme that can hydrolyze the β-glycosidic bond in lactose to generate galactose (e.g., β-D-galactose) and glucose (e.g., D-glucose). The “lactase” catalyzed hydrolysis of lactose is schematically depicted in FIG. 1.

The term “lactose” refers to a disaccharide that has the molecular formula C₁₂H₂₂O₂₁, and that consists of a β-D-galactose molecule and a D-glucose molecule bonded through a β1-4 glycosidic linkage. The structure of “lactose”, and its hydrolysis to β-D-galactose and D-glucose, is shown in FIG. 1.

The term “MEV pathway” refers to a biosynthetic pathway for the conversion of acetyl-CoA into isopentenyl diphosphate isomerase (“IPP”). Enzymes of the MEV pathway include an enzyme that can convert two molecules of acetyl-coenzyme A into acetoacetyl-CoA, an enzyme that can convert acetoacetyl-CoA and acetyl-coenzyme A into 3-hydroxy-3-methylglutaryl-CoA (HMG-CoA), an enzyme that can convert HMG-CoA into mevalonate, an enzyme that can convert mevalonate into mevalonate 5-phosphate, an enzyme that can convert mevalonate 5-phosphate into mevalonate 5-pyrophosphate, and an enzyme that can convert mevalonate 5-pyrophosphate into IPP.

The term “nucleotide sequence” refers to the order of nucleic acid bases in a DNA or RNA strand.

The term “operably linked” refers to a juxtaposition wherein the components so described are in a relationship permitting them to function in their intended manner. For instance, a promoter is operably linked to a protein coding sequence if the promoter affects the transcription into MnRtNA of the protein coding sequence.

The term “prenyl diphosphate synthase” refers to an enzyme that can convert isopentenyl diphosphate isomerase (“IPP”) and/or dimethylallyl pyrophosphate (“DMAPP”) into a prenyl diphosphate. Examples of prenyl diphosphates are farnesyl diphosphate (“FPP”), geranyl diphosphate (“GPP”), and geranylgeranyl diphosphate (“GGPP”).

The term “protein coding sequence” refers to a nucleotide sequence that encodes a protein.

The term “substantially pure” refers to substantially free of one or more other compounds, i.e., the composition contains greater than 80 volume %, greater than 90 volume %, greater than 95 volume %, greater than 96 volume %, greater than 97 volume %, greater than 98 volume %, greater than 99 volume %, greater than 99.5 volume %, greater than 99.6 volume %, greater than 99.7 volume %, greater than 99.8 volume %, or greater than 99.9 volume % of the compound; or less than 20 volume %, less than 10 volume %, less than 5 volume %, less than 3 volume %, less than 1 volume %, less than 0.5 volume %, less than 0.1 volume %, or less than 0.01 volume % of the one or more other compounds, based on the total volume of the composition.

The term “recombinant” refers to a particular nucleic acid (DNA or RNA) is the product of various combinations of cloning, restriction, and/or ligation steps resulting in a construct having a structural coding or non-coding sequence distinguishable from endogenous nucleic acids found in natural systems.

The term “regulatory element” refers to transcriptional and translational control sequences, such as promoters, enhancers, polyadenylation signals, terminators, protein degradation signals, and the like, that provide for and/or regulate expression of a transcript, a coding sequence and/or production of an encoded polypeptide in a cell.

The term “signal peptide” refers to a segment of the amino acid sequence of a protein that mediates secretion of the protein from a cell.

The term “terpene synthase” refers to an enzyme that can convert one or more prenyl pyrophosphates into an isoprenoid.

A polynucleotide or polypeptide has a certain percent “sequence identity” to another polynucleotide or polypeptide, meaning that, when aligned, that percentage of bases or amino acids are the same, and in the same relative position, when comparing the two sequences. To determine sequence identity, sequences can be aligned using methods and computer programs widely available to the public, including BLAST (available over the world wide web at ncbi.nlm.nih.gov/BLAST), FASTA (available in the Genetics Computing Group (GCG) package, Madison, Wis.), Smith-Waterman algorithm, Needleman and Wunsch alignment, and other techniques.

The term “transporter” refers to a protein that mediates the transfer of a compound across a cell membrane or membrane of a cellular organelle.

The terms “polypeptide”, “peptide”, “amino acid sequence” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component. As used herein the term “amino acid” refers to either natural and/or unnatural or synthetic amino acids, including but not limited to glycine and both the D or L optical isomers, and amino acid analogs and peptidomimetics.

### Inducible Expression of Heterologous Sequences

The present invention provides compositions and methods for expressing heterologous sequences resulting in heterologous products in a host cell. In one aspect, the heterologous sequence is operably linked to a galactose-inducible regulatory element, but expression of which is induced without directly supplementing galactose to the culture medium. Induction occurs by the addition of one or more compounds, typically lactose, which can be broken down into galactose, whereby the resulting galactose induces the expression of the heterologous sequences. In other embodiments, expression of the heterologous sequence is induced upon expression of lactase which hydrolyzes lactose present in the medium to generate galactose, which in turn activates expression of the heterologous sequence of interest. The expression of the heterologous sequence can be induced to a level comparable to that obtained by culturing the host cell in a medium supplemented with comparable quantities (as measured in moles) of galactose. In particular, the amount of heterologous product produced by a host cell culture in medium supplemented with lactose is comparable to that produced in a medium supplemented with same or comparable moles of galactose.

In another embodiment, the culture medium further comprises an enzyme that hydrolyzes lactose into galactose, such as lactase or a biologically active fragment thereof. The enzyme can be produced by the host cell that carries the heterologous sequence to be expressed. For example the host cell may produce endogenous lactase or produce lactase from a heterologous nucleic acid sequence. Where desired, the lactase produced is secreted into the cell culture medium. In yet another embodiment, the lactase can be produced by another cell that does not carry the heterologous sequence of interest but are used to supply lactase or biologically active fragment thereof for generating galactose, which in turn activates the expression of the heterologous sequence.

In still other embodiments, expression of the heterologous sequence is induced upon the addition of exogenous lactase to the medium comprising the host cells and lactose.

When the lactose is converted into galactose outside of the host cells comprising the heterologous sequence (e.g. in the medium), galactose generated from lactose can be imported into the host cell by a galactose transporter. This can be carried out by an endogenous galactose transporter or a heterogenous galactose transporter. The imported galactose can then induce the one or more heterologous sequences operably linked to a galactose-inducible regulatory element in the cell.

In yet other embodiments, lactose supplemented to the medium can be transported into the host cell, where it is hydrolyzed inside the cell by endogenous lactase or lactase expressed by a heterologous sequence. The hydrolysis of lactose inside the cell yields glucose and galactose, the latter being utilized to activate expression of the heterologous sequence of interest that is operably linked to a galactose-inducible regulatory element. Suitable lactose transporter again can be endogenous or exogenous, e.g., an exogenous lactase that is expressed by a heterologous sequence.

### Galactose Induction Machinery

The host cell of the present invention comprises a galactose-induction machinery. The galactose induction machinery may be endogenous (e.g., as in Saccharomyces cerevisiae) or heterologous to the host cell. The galactose induction machinery refers to the collection of proteins that induces transcription of a heterologous sequence operably linked a galactose-inducible regulatory element in the presence of galactose. An example of a galactose induction machinery is the collection of yeast proteins Gal3p, Gal4p, and Gal80p, or functional homologs thereof including biologically active fragments thereof. Suitable nucleotide sequences for use in the present invention in generating host cells comprising a heterologous galactose induction machinery include but are not limited to the nucleotide sequences of the Gal4 gene of Saccharomyces cerevisiae (GenBank locus tag YPL248C), the Gal80 gene of Saccharomyces cerevisiae (GenBank locus tag YML051W), and the Gal3 gene of Saccharomyces cerevisiae (GenBank locus tag YDR009W), and their functional homologs.

The host cell of the present invention further comprises a galactose-inducible regulatory element. The regulatory element can be transcriptional or translational control sequences, such as promoters, enhancers, polyadenylation signals, terminators, protein degradation signals, and the like, that provide for and/or regulate expression of a transcript, a coding sequence and/or production of an encoded polypeptide in a cell. The galactose-inducible regulatory element can be endogenous or heterologous. For example, the host cell may comprise a single heterologous galactose-inducible expression cassette, wherein the galactose-inducible expression cassette comprises a galactose-inducible regulatory element. A single heterologous galactose-inducible expression cassette can express one or more heterologous sequences of the same or different sequence identity. In some embodiments, the expression cassette may drive the expression of multiple copies of the same or different heterologous sequences. In some embodiments, the single heterologous galactose-inducible expression cassette can express 2, 3, 4, 5 or copies of the same or different heterologous sequences. In one embodiments, the expression vector may comprise a first heterologous sequence operably linked to a galactose-inducible regulatory element and a second heterologous sequence encoding a lactase or biologically active fragment thereof. Where desired, a single expression cassette can drive the expression of heterologous sequences encoding 2, 3, 4, 5, or more different proteins of a biochemical pathway, such as the MEV or DXP pathway. For example, a single expression cassette can encode both HMGCoA reductase and another enzyme, such as farnesyl diphosphate synthase, isopentyl δ isomerase. In other embodiments, a single expression cassette control expression of mevalonate kinase and acetoacetyl CoA thiolase or diphosphoemevalonate decarboxylase and phosphomevalonate kinase. The expression cassette for expression of any combinations of enzymes in a given pathway can be constructed according to routine recombinant procedures.

The host cell can also comprise a plurality of heterologous galactose-inducible expression cassettes. For example, the host cell can have multiple expression cassettes that control the expression of the same or different heterologous sequences. Where desired, each of the multiple expression cassettes can be designed to control the expression of the same protein, a different protein. Alternatively, a subset of the plurality of heterologous galactose-inducible expression cassettes can be utilized to drive expression of the same protein and another subset expresses different proteins. Furthermore, the host cell can comprise other exogenous sequences that modulate the expression of the heterologous sequence of interest. Depending on the choice of the heterologous product that is to be produced, the other exogenous sequences can encompass lactase, especially a secretable lactase to facilitate the hydrolysis of lactose supplemented to the cell culture medium. Other non-limiting examples include exogenous sequences encoding lactose transporter, galactose transporter and functional homologos. These and other suitable exogenous sequences can be constitutively expressed or be placed under the control of a non-galactose inducible regulatory element.

The subject galactose-inducible regulatory element encompasses a galactose-inducible promoter. Inducible promoters are typically used instead of constitutive promoters in the herelogous production of proteins because the former permits control of protein production at physiologically optimal time points and/or levels (e.g., levels that are not toxic to the physiological state of the cell). Galactose-inducible promoters are frequently used in the heterologous production of proteins because thye are amenable to targeted and tight regulation, and provide high levels of expression. Suitable galactose-inducible promoters for use in the present invention include but are not limited to the promoters of the Saccharomyces ceverisiae genes GAL7 (GenBank accession NC_{—}001134 REGION: 274427 . . . 275527), GAL2 (GenBank accession NC_{—}001144 REGION: 290213 . . . 291937), GAL1 (GenBank accession NC_{—}001134 REGION: 279021 . . . 280607), GAL10 (GenBank accession NC_{—}001134 REGION: 276253 . . . 278352), GAL3 (GenBank accession NC 001136 REGION: 463431 . . . 464993), GCY1 (GenBank accession NC_{—}001147 REGION: 551115 . . . 552053), and GAL80 (GenBank accession NC_{—}001145 REGION: 171594 . . . 172901) or functional homologs thereof. In certain embodiments, the galactose-inducible promoter comprises the nucleotide sequence CG(G or C)(N₁₁)(G or C)CG, where N is any nucleotide. Hybrid promoters may also be used, for example, as disclosed in U.S. Pat. No. 5,739,007, U.S. Pat. No. 5,310,660 or U.S. Pat. No. 5,013,652. In certain embodiments, the galactose-inducible promoter is a synthetic promoter (i.e., the promoter is synthesized chemically).

In certain embodiments, the galactose-inducible promoter provides for high-level transcription of a given heterologous sequence. In other embodiments, the galactose-inducible promoter provides for low-level transcription of the heterologous sequence. A number of genes are induced in the presence of galactose (Ren et al., Genome-wide location and function of DNA binding proteins. Science 290:2306-2309 (2000)). Promoters for these genes, such as UAS_{GAL} may also have differential activation levels. For example, without being bound to theory, a number of UAS_{GAL} have been identified in yeast, and have different relative affinities for Gal4p and thus, differential activation (see for example, Lohr et al., Transcriptional regulation in the yeast GAL gene family: a complex genetic network. FASEB J 9:777-787 (1995)). These and any other variant promoters are encompassed as galactose-inducible regulatory elements for fine-tuning the desired expression levels when practicing the subject methods.

### Culture Medium

Expression of a heterologous sequence typically involves culturing a host cell comprising such heterologous sequence in a culture medium. A suitable culture medium encompasses any medium that provides for growth or maintenance of a host cell culture. The general parameters governing prokaryotic and eukaryotic cell survival are well established in the art, Physicochemical parameters which may be controlled in vitro are, e.g., pH, CO₂, temperature, and osmolarity. The nutritional requirements of cells are usually provided in standard media formulations developed to provide an optimal environment. Nutrients can be divided into several categories: amino acids and their derivatives, carbohydrates, sugars, fatty acids, complex lipids, nucleic acid derivatives and vitamins. Apart from nutrients for maintaining cell metabolism, some cells may require one or more hormones from at least one of the following groups: steroids, prostaglandins, growth factors, pituitary hormones, and peptide hormones to survive or proliferate (Sato, G. H., et al. in “Growth of Cells in Hormonally Defined Media”, Cold Spring Harbor Press, N.Y., 1982; Ham and Wallace (1979) Meth. Enz., 58:44, Barnes and Sato (1980) Anal Biochem., 102:255, or Mather, J. P. and Roberts, P. E. (1998) “Introduction to Cell and Tissue Culture”, Plenum Press, New York.

A suitable culture medium typically comprises a readily available source of energy (e.g., a simple sugar such as glucose, galactose, mannose, fructose, ribose, or combinations thereof), a nitrogen source, and a phosphate source. In certain embodiments, the culture medium is a liquid medium. Suitable liquid media include but are not limited to: YPD (YEPD), YPAD, Hartwell's complete (HC), and synthetic complete (SC) media. In certain embodiments, the culture medium is supplemented with one or more additional agents (e.g., an inducer other than galactose when the production of the galactose transporter, lactose transporter, or lactase in the cell is under control of an inducible promoter). In other embodiments, the culture medium is supplemented with both lactose and galactose in various proportions to yield a desired induction level. Where desired, a “defined medium” can be employed for culturing the host cells. A defined medium typically comprises nutritional and other requirements necessary for the survival and/or growth of the cells in culture such that the components of the medium are known. Traditionally, the defined medium has been formulated by the addition of nutritional and/or growth factors necessary for growth and/or survival. Typically, the defined medium provides at least one component from one or more of the following categories: a) all essential amino acids, and usually the basic set of twenty amino acids plus cystine; b) an energy source, usually in the form of a carbohydrate such as glucose; c) vitamins and/or other organic compounds required at low concentrations; d) free fatty acids; and e) trace elements, where trace elements are defined as inorganic compounds or naturally occurring elements that are typically required at very low concentrations, usually in the micromolar range. The defined medium may also optionally be supplemented with one or more components from any of the following categories: a) one or more mitogenic agents; b) salts and buffers as, for example, calcium, magnesium, and phosphate; c) nucleosides and bases such as, for example, adenosine and thymidine, hypoxanthine; and d) protein and tissue hydrolysates.

Culturing the host cell in a medium can occur in any vessel or on any substrate that maintains cell viability and/or growth. Suitable vessels include but are not limited to a tank for a reactor or fermentor, or a part of a centrifuge that can separate heavier materials from lighter materials in subsequent processing steps. In certain embodiments, the vessel has a capacity of at least 1 liter. In some such embodiments, the vessel has a capacity of at least 10 liter. In some such embodiments, the vessel has a capacity of at least 100 liter. In some embodiments, the vessel has a capacity of from 100 to 3,000,000 liters such as at least 1000 liters, at least 5,000 liters, at least 10,000 liters, vessel at least 25,000 liters, at least 50,000 liters, at least 75,000 liters, at least 100,000 liters, at least 250,000 liters, at least 500,000 liters or at least 1,000,000 liters.

The culture medium of the invention comprises one or more compounds that can be broken down into galactose. In methods of the present invention, the medium typically comprises lactose. Lactose can be hydrolyzed into galactose and glucose and is a relatively cheap compound, typically costing significantly less than galactose, as lactose is the major constituent of whey, which is a waste product of many commercial dairy product manufacturing processes. Given the low cost of lactose, and the availability of enzymes that can hydrolyze lactose, enzymatic hydrolysis of lactose presents a cost-effective means for generating galactose for the induction of galactose-inducible expression systems for the large-scale production of proteins.

In certain embodiments, the lactose concentration in the culture medium is less than 10 g/L, less than 5 g/L, or less than 2 g/L. In certain embodiments, the lactose is added to the medium as a substantially pure compound. In other embodiments, the lactose is added to the medium as a component of a mixture of compounds. In some embodiments, the lactose is added to the medium as a component of whey. In other embodiments, the lactose is added to the medium as a component of milk or a milk product. In yet other embodiments, the lactose is secreted into the culture medium by the host cell. In other embodiments, the lactose is secreted into the culture medium by a cell other than the host cell. In certain embodiments, the lactose is generated in the culture medium through the action of certain enzymes that are present in the culture medium. In certain such embodiments, the enzymes are added to the culture medium in substantially pure form. In other such embodiments, the enzymes are added to the culture medium as components of a mixture of enzymes. In other such embodiments, the enzymes are secreted by the host cell. In still other such embodiments, the enzymes are secreted by a cell other than the host cell. The enzymes can be present in the medium from a combination of the aforementioned methods, for example, added in substantially pure form and also secreted by a host cell and/or a cell that is not the host cell.

In some embodiments, the culture medium of the invention also comprises an enzyme that hydrolyzes lactose to galactose and glucose. The enzyme can be a lactase. Suitable lactases for use in the present invention include but are not limited to (GenBank Accession number; organism): LAC4 (M84410 REGION: 43 . . . 3120; Khuyveromyces lactis), lacZ (X91197, Escherichia coli), LacA (S37150; Aspergillus niger), and other members of Enzyme Commission class 3.1.1.23. Functional variants may also be used. In certain embodiments, the lactase is added to the medium as a substantially pure enzyme. Substantially pure lactase for use in the invention can, for example, be obtained by pulverizing commercially available lactose tablets (e.g., the Dairy Digestive supplement available from Long's Drugstore). In other embodiments, the lactase is added to the medium as a component of a mixture of enzymes and/or compounds.

In certain embodiments, lactase is secreted into the culture medium by the host cell or by a cell other than the host cell. In certain embodiments, the lactase is released into the culture medium by virtue of comprising a native signal peptide that mediates the enzyme's transport out of a cell. Suitable secreted lactases that comprise a native signal peptide include but are not limited to LacA (S37150; Aspergillus niger). In other embodiments, the lactase is released into the culture medium by virtue of being fused to a heterologous signal peptide that mediates the enzyme's transport out of a cell. Suitable signal peptides include but are not limited to the signal peptides of the Saccharomyces cerevisiae alpha-mating factor and the Kluyveromyces lactis killer toxin. In certain embodiments, the lactase is released into the culture medium as a result of cell lysis. Cell lysis may occur, for example, in a high density cell culture or as a result of the expression in a cell of the invention of a heterologous protein (Compagno et al. (1995) Appl. Microbiol. Biotechnol. 43(5):822-825).

Lactase produced in the host cell or in a cell other than the host cell that is secreted may be endogenously produced or heterologously produced. Production of lactase in the host cell or in a cell other than the host cell may be controlled by a promoter. The promoter may be constitutive or inducible. Suitable inducible promoters include but are not limited to the promoters of the Saccharomyces cerevisiae genes ADH2, PHO5, CUP1, MET2S, MET3, CYC1, HIS3, GAPDH, ADC1, TRP1, URA3, LEU2, TP1, and AOX1. In other embodiments, the promoter is constitutive. Suitable constitutive promoters include but are not limited to Saccharomyces cerevisiae genes PGK1, YDH1, YDH3, FBA1, ADH1, LEU2, ENO, TPI1, and PYK1.

### Lactase, Lactose Transporters, and Galactase Transporters

In certain embodiments, the host cell of the invention comprises a lactase, or biologically active fragments thereof, that can hydrolyze lactose into galactose and glucose (FIG. 1). The lactase may be endogenous to the host cell or heterologous, for example, produced from a heterologous nucleic acid sequence. In some embodiments, the lactase is secreted from the host cell into the medium. A secretable lactase typically comprises a signal peptide that is cleaved post-translationally. Alternatively, the endogenous or heterologous lactase may reside within the cell and hydrolyzes lactose that is imported into the cell via e.g., a lactose transporter. Suitable lactases include but are not limited to (GenBank Accession number; organism): LAC4 (M84410 REGION: 43 . . . 3120; Kluyveromyces lactis), lacZ (X91197; Escherichia coli), LacA (S37150; Aspergillus niger), and other members of Enzyme Commission number 3.1.1.23. In certain embodiments, the amino acid sequence of the lactase comprises SEQ ID NO: 3, or a variant thereof. In certain embodiments, the nucleotide sequence encoding the lactase comprises SEQ ID NO: 4, or a homolog thereof.

Production of lactase in the host cell may be controlled by a promoter. In certain embodiments, the promoter is inducible. Suitable inducible promoters include but are not limited to the promoters of the Saccharomyces cerevisiae genes ADH2, PHO5, CUP1, MET25, MET3, CYC1, HIS3, GAPDH, ADC1, TAP1, URA3, LEU2, TP1, and AOX1. In other embodiments, the promoter is constitutive. Suitable constitutive promoters include but are not limited to Saccharomyces cerevisiae genes PGK1, TDH1, TDH3, FBA1, ADR1, LEU2, ENO, TPI1, and PYK1.

In certain embodiments, the host cell of the invention comprises a lactose transporter that can import lactose from the culture medium into the cytosol of the cell. For example, if lactose is present in the medium and lactase is present in the host cell, the host cell comprises a lactose transporter. The lactose transporter may be endogenous or heterologous. In some embodiments, a host cell may comprise both endogenous and heterologous lactose transporters. Suitable lactose transporters include but are not limited to: LAC12 (SenBank accession no. X06997 REGION: 1616 . . . 3379; Kluyveromyces lactis) and LacY (GenBank Locus Tag B0343; Escherichia coli). In certain embodiments, the amino acid sequence of the lactose transporter comprises SEQ ID NO: 1, or a variant thereof. In certain embodiments, the nucleotide sequence encoding the lactose transporter comprises SEQ ID NO: 2, or a homolog thereof.

In certain embodiments, the host cell of the invention comprises a galactose transporter that can import galactose from the culture medium into the cytosol of the cell. For example, a host cell that expresses a galactose transporter is cultured in media comprising lactose and lactase, which permits galactose to be imported into the host cell. The galactose transporter may be endogenous or may be heterologous, for example, expressed from a heterologous nucleotide sequence. The host cell may comprise both endogenous and heterologous galactose transporters. Suitable galactose transporters include but are not limited to: GAL2 (GenBank Locus Tag YLR081W; Saccharomyces cerevisiae), MST4 (AY342321; Oryza sativa Japonica Group), MST4 (DQ087177; Olea europaea), LAC12 (X06997; Kluyveromyces lactis), GAL2 (AAU43755; Saccharomyces mikatae), and HGT1 (KLU22525; Kluyveromyces lactis).

Production of the lactose transporter or galactose transporter in the host cell may be controlled by a promoter. In certain embodiments, the promoter is inducible. Suitable inducible promoters include but are not limited to the promoters of the Saccharomyces cerevisae genes ADH2, PH05, CUP1, MET25, MET3, CYC1, HIS3, GAPDH, ADC1 TR1, URA3, LEU2, TP1, and AOX1. In other embodiments, the promoter is constitutive. Suitable constitutive promoters include but are not limited to Saccharomyces cerevisiae genes PGK1, TDH1, TDH3, FBA 1, ADH1, LEU2, ENO, TPI1, and PYK1.

### Heteroloaous Products

The compositions of the present invention including without limitation vectors, host cells, culture media and galactose-inducible regulatory elements, are suitable for expression of any heterologous sequences in an inducible manner. To induce production of any of the heterologous products, an inducing agent typically a non-galactose sugar is employed. The amount of product produced by host cells cultured in a medium supplemented with lactose can be comparable to the amount of product produced from a culture medium supplemented with a comparable quantity of galactose. In some embodiments, the amount of heterologous product produced is approximately equal to or greater than the amount of product produced from the same host cell upon adding the same quantity of galactose directly into the medium. In some embodiments, the amount of product produced is at least about 1.2 fold, 1.5 fold, 2 fold, 2.5 fold, 3 fold, 4, fold, 5 fold or more than the amount of product produced by adding the same quantity of galactose to the medium.

The heterologous sequence to be expressed can encode a protein or peptide, such as bioactive proteins or peptides. Depending on the nature of the protein, it can be utilized by a host cell for the synthesis or breakdown of lipids, carbohydrates, and combinations thereof. Expression of the heterologous sequences can yield nucleic acid products including but not liinted to oligonucleotides, e.g., ribonucleotides, antisense molecules, RNAi molecules, ribozymes, external-guided sequences (EGS), aptamers, and miRNA.

For example, the heterologous sequences to be expressed by the subject compositions or via the subject methods encompass several classes of catalytic RNAs (ribozymes), including intron-derived ribozymes (WO 88/04300; see also, Cech, T., Annu. Rev. Biochem., 59:543-568, (1990)), hammerhead ribozymes (WO 89/05852 and EP 321021), axehead ribozymes (WO 91/04319 and WO 91/04324) and any other heterologous sequences exemplified herein. EGS molecules may also be encoded by heterologous sequences of the present invention when operably linked to a galactose-inducible regulatory element. EGS typically binds to a target substrate to form a secondary and tertiary structure resembling the natural cleavage site of precursor tRNA for eukaryotic RNAse P. Methods of designing EGS molecules are described, for example in U.S. Pat. No. 5,624,824, U.S. Pat. No. 5,683,873, U.S. Pat. No. 5,728,521, U.S. Pat. No. 5,869,248, U.S. Pat. No. 5,877,162, and U.S. Pat. No. 6,057,153, all of which are incorporated herein in their entirety.

Heterologous sequences may also produce antisense molecules, siRNA, miRNA, and aptamers. The design of heterologous sequences that produce siRNA, antisense molecules, EGS, or miRNA, generally requires knowledge of the mRNA primary sequence of a cellular target. Primary mRNA sequence information of the entire mouse and human genome, as well as the gene sequences from a number of other organisms including avian, canine, feline, rattus, and others are readily available to the public on the NCBI server, www.ncbi.nlm.nih-gov. Standard methods in the design of siRNA are known in the art (Elbashir et al., Methods 26:199-213 (2002)) and public design tools are also readily available, for example, from the Whitehead Institute of Biomedical Research at MIT, http://jura.wi.mit.edu/pubint/http://iona.wi.mit.edu/siRtNAext/ and www.RNAinterference.org, as well as from commercial sites from Promega and Ambion. Databases of miRNA sequences are also publicly available, such as at http://www.microrna.org/ and http://microrna.sanger.ac.uk/. Aptamers may be generated by methods known in the art or sequences obtained from a public database such as http://aptamer.icmb.utexas.edu.

The heterologous sequence may also encode a proteinaceous product, such as a protein or a peptide. The protein may be endogenous or exogenous to the cell. The protein may be an intracellular protein (e.g., a cytosolic protein), a transmembrane protein, or a secreted protein. Heterologous production of proteins is widely employed in research and industrial settings, for example, for production of therapeutics, vaccines, diagnostics, biofuels, and many other applications of interest. Exemplary therapeutic proteins that can be produced by employing the subject compositions and methods include but are not limited to certain native and recombinant human hormones (e.g., insulin, growth hormone, insulin-like growth factor 1, follicle-stimulating hormone, and chorionic gonadotropin), hematopoietic proteins (e.g., erycbropoietin, C-CSF, GM-CSF, and IL-11), thrombotic and hematostatic proteins (e.g., tissue plasminogen activator and activated protein C), immunological proteins (e.g., interleukin), and other enzymes (e.g., deoxyribonuclease I). Examplary vaccines that can be produced by the subject compositions and methods include but are not limited to vaccines against various influenza viruses (e.g., types A, B and C and the various serotypes for each type such as H5N2, H1N1, H3N2 for type A influenza viruses), HIV, hepatitis viruses (e.g., hepatitis A, B, C or D), Lyme disease, and human papillomavirus (HPV). Examples of heterologously produced protein diagnostics include but are not limited to secretin, thyroid stimulating hormone (TSH), HIV antigens, and hepatitis C antigens.

Proteins or peptides produced by the heterologous sequence can include, but are not limited to cytokines, chemokines, lymphokines, ligands, receptors, hormones, enzymes, antibodies and antibody fragments, and growth factors. Non-limiting examples of receptors include TNF type I receptor, IL-1 receptor type II, IL-1 receptor antagonist, IL-4 receptor and any chemically or genetically modified soluble receptors. Examples of enzymes include lactase, activated protein C, factor VII, collagenase (e.g., marketed by Advance Biofactures Corporation under the name Santyl); agalsidase-β (e.g., marketed by Genzyme under the name Fabrazyme); dornase-α (e.g., marketed by Genentech under the name Pulmozyme); alteplase (e.g., marketed by Genentech under the name Activase); pegylated-asparaginase (e.g., marketed by Enzon under the name Oncaspar); asparaginase (e.g., marketed by Merck under the name Elspar); and imiglucerase (e.g., marketed by Genzyme under the name Ceredase). Examples of specific polypeptides or proteins include, but are not limited to granulocyte macrophage colony stimulating factor (GM-CSF), granulocyte colony stimulating factor (G-CSF), macrophage colony stimulating factor (M-CSF), colony stimulating factor (CSF), interferon beta (IFN-β), interferon gamma (IFNγ), interferon gamma inducing factor I (IGIF), transforming growth factor beta (IGF-β), RANTES (regulated upon activation, normal T-cell expressed and presumably secreted), macrophage inflammatory proteins (e.g., MIP-1-α and MIP-1-β), Leishmnania elongation initiating factor (LEIF), platelet derived growth factor (PDGF), tumor necrosis factor (TNF), growth factors, e.g., epidermal growth factor (EGF), vascular endothelial grouth factor (VEGF), fibroblast growth factor, (FGF), nerve growth factor (NGF), brain derived neurotrophic factor (BDNF), neurotrophin-2 (NT-2), neurotrophin-3 (NT-3), neurotrophin-4 (NT-4), neurotrophin-5 (NT-5), glial cell line-derived neurotrophic factor (GDNF), ciliary neurotrophic factor (CNTF), TNF α type II receptor, erythropoietin (EPO), insulin and soluble glycoproteins e.g., gp120 and gp160 glycoproteins. The gp120 glycoprotein is a human immunodeficiency virus (WIV) envelope protein, and the gp160 glycoprotein is a known precursor to the gp120 glycoprotein. Other examples include secretin, nesiritide (human B-type natriuretic peptide (hBNP)), GYP-I .

Other heterologous products may include GPCRs, including, but not limited to Class A Rhodopsin like receptors such as Muscatinic (Muse.) acetylcholine Vertebrate type 1, Musc. acetylcholine Vertebrate type 2, Musc. acetylcholine Vertebrate type 3, Musc. acetylcholine Vertebrate type 4; Adrenoceptors (Alpha Adrenoceptors type 1, Alpha Adrenoceptors type 2, Beta Adrenoceptors type 1, Beta Adrenoceptors type 2, Beta Adrenoceptors type 3, Dopamine Vertebrate type 1, Dopamine Vertebrate type 2, Dopamine Vertebrate type 3, Dopamine Vertebrate type 4, Histamine type 1, Histamine type 2, Histamine type 3, Histamine type 4, Serotonin type 1, Serotonin type 2, Serotonin type 3, Serotonin type 4, Serotonin type 5, Serotonin type 6, Serotonin type 7, Serotonin type 8, other Serotonin types, Trace amine, Angiotensin type 1, Angiotensin type 2, Bombesin, Bradykffin, C5a anaphylatoxin, Finet-leu-phe, APJ like, Interleukin-8 type A, Interleukin-8 type B, Interleukin-8 type others, C-C Chemokine type 1 through type 11 and other types, C—X—C Chemokine (types 2 through 6 and others), C-X3-C Chemokine, Cholecystokinin CCK, CCK type A, CCK type B, CCK others, Endothelin, Melanocortin (Melanocyte stimulating hormone, Adrenocorticotropic hormone, Melanocortin hormone), Duffy antigen, Prolactin-releasing peptide (GPR10), Neuropeptide Y (type 1 through 7), Neuropeptide Y, Neuropeptide Y other, Neurotensin, Opioid (type D, K, M, X), Somatostatin (type 1 through 5), Tachykinin (Substance P(NK1), Substance K (NK2), Neuromedin K (NK3), Tachykinin like 1, Tachykinin like 2, Vasopressin/vasotocin (type 1 through 2), Vasotocin, Oxytocin/mesotocin, Conopressin, Galanin like, Proteinase-activated like, Orexin & neuropeptides FF, QRFP, Chemokine receptor-like, Neuromedin U like (Neuromedin U, PRXamide), hormone protein (Follicle stimulating hormone, Lutropin-choriogonadotropic hormone, Thyrotropin, Gonadotropin type I, Gonadotropin type II), (Rhod)opsin, Rhodopsin Vertebrate (types 1-5), Rhodopsin Vertebrate type 5, Rhodopsin Arthropod, Rhodopsin Arthropod type 1, Rhodopsin Arthropod type 2, Rhodopsin Arthropod type 3, Rhodopsin Mollusc, Rhodopsin, Olfactory (Olfactory 11 fam 1 through 13), Prostaglandin (prostaglandin E2 subtype EP 1, Prostaglandin E2/D2 subtype EP2, prostaglandin E2 subtype EP3, Prostaglandin E2 subtype EP4, Prostaglandin F2-alpha, Prostacyclin, Thromboxane, Adenosine type 1 through 3, Purinoceptors, Purinoceptor P2RY1-4,6,11 GPR91, Purinoceptor P2RY5,8,9,10 GPR35,92,174, Purinoceptor P2RY12-14 GPR87 (JDP-Glucose), Cannabinoid, Platelet activating factor, Gonadotropin-releasing hormone, Gonadotropin-releasing hormone type I, Gonadotropin-releasing hormone type II, Adipokinetic hormone like, Corazonin, Thyrotropin-releasing hormone & Secretagogue, Thyrotropin-releasing hormone, Growth hormone secretagogue, Growth hormone secretagogue like, Ecdysis-triggering hormone (ETHR), Melatonin, Lysosphingolipid & LPA (EDG), Sphingosine 1-phosphate Edg-1, Lysophosphatidic acid Edg-2, Sphingosine 1-phosphate Edg-3, Lysophosphatidic acid Edg4, Sphingosine 1-phosphate Edg-5, Sphingosine 1-phosphate Edg-6, Lysophosphatidic acid Edg-7, Sphingosine 1-phosphate Edg-8, Edg Other Leukotriene B4 receptor, Leukotriene B4 receptor BLT1, Leukotriene B4 receptor BLT2, Class A Orphan/other, Putative neurotransmitters, SREB, Mas proto-oncogene & Mas-related (MRGs), GPR45 like, Cysteinyl leukotriene, G-protein coupled bile acid receptor, Free fatty acid receptor (GP40, GP41, GP43), Class B Secretin like, Calcitonin, Corticotropin releasing factor, Gastric inhibitory peptide, Glucagon, Growth hormone-releasing hormone, Parathyroid hormone, PACAP, Secretin, Vasoactive intestinal polypeptide, Latrophilin, Latrophilin type 1, Latrophilin type 2, Latrophilin type 3, ETL receptors, Brain-specific angiogenesis inhibitor (BAI), Methuselah-like proteins (MTH), Cadherin EGF LAG (CELSR), Very large G-protein coupled receptor, Class C Metabotropic glutamate/pheromone, Metabotropic glutamate group I through III, Calcium-sensing like, Extracellular calcium-sensing, Pheromone, calcium-sensing like other, Putative pheromone receptors, GABA-B, GABA-B subtype 1, GABA-B subtype 2, GABA-B like, Orphan GPRC5, Orphan GPCR6, Bride of sevenless proteins (BOSS), Taste receptors (TiR), Class D Fungal pheromone, Fungal pheromone A-Factor like (STE2,STE3), Fungal pheromone B like (BAR,BBR,RCB,PRA), Class E cAMP receptors, Ocular albinism proteins, Frizzled/Smoothened family, frizzled Group A (Fz 1&2&4&5&7-9), frizzled Group B (Fz 3 & 6), fizzled Group C (other), Vomeronasal receptors, Nematode chemoreceptors, Insect odorant receptors, and Class Z Archaeal/bacterial/fungal opsins.

Bioactive peptides may also be produced by the heterologous sequences of the present invention. Examples include: BOTOX, Myobloc, Neurobloc, Dysport (or other serotypes of botulinum neurotoxins), alglucosidase alfa, daptomycin, YH-16, choriogonadotropin alfa, filgrastim, cetrorelix, interleukin-2, aldesleukin, teceleulin, denileukin diftitox, interferon alfa-n3 (injection), interferon alfa-nl, DL-8234, interferon, Suntory (gamma-1a), interferon gamma, thymosin alpha 1, tasonermin, DigiFab, ViperaTAb, EchiTAb, CroFab, nesiritide, abatacept, alefacept, Rebif, eptoterminalfa, teriparatide (osteoporosis), calcitonin injectable (bone disease), calcitonin (nasal, osteoporosis), etanercept, hemoglobin glutamer 250 (bovine), drotrecogin alfa, collagenase, carperitide, recombinant human epidermal growth factor (topical gel, wound healing), DWP401, darbepoetin alfa, epoetin omega, epoetin beta, epoetin alfa, desirudin, lepirudin, bivalirudin, nonacog alpha, Mononine, eptacog alfa (activated), recombinant Factor VIII+VWF, Recombinate, recombinant Factor VIII, Factor VIII (recombinant), Alphnmate, octocog alfa, Factor VIII, palifermin, Indikinase, tenecteplase, alteplase, pamiteplase, reteplase, nateplase, monteplase, follitropin alfa, rFSH, hpFSH, micafungin, pegfilgrastim, lenograstim, nartograstim, sermorelin, glucagon, exenatide, pramlintide, iniglucerase, galsulfase, Leucotropin, molgramostirn, triptorelin acetate, histrelin (subcutaneous implant, Hydron), deslorelin, histrelin, nafarelin, leuprolide sustained release depot (ATRIGEL), leuprolide implant (DUROS), goserelin, somatropin, Eutropin, KP-102 program, somatropin, somatropin, mecasermin (growth failure), enlfavirtide, Org-33408, insulin glargine, insulin glulisine, insulin (inhaled), insulin lispro, insulin deternir, insulin (buccal, RapidMist), mecasermin rinfabate, anakinra, celmoleukin, 99 mTc-apcitide injection, myelopid, Betaseron, glatiramer acetate, Gepon, sargramostim, oprelvekin, human leukocyte-derived alpha interferons, Bilive, insulin (recombinant), recombinant human insulin, insulin aspart, mecasenin, Roferon-A, interferon-alpha 2, Alfaferone, interferon alfacon-1, interferon alpha, Avonex′ recombinant human luteinizing hormone, dornase alfa, trafermin, ziconotide, taltirelin, diboterminalfa, atosiban, becaplermin, eptifibatide, Zemaira, CTC-111, Shanvac-B , HPV vaccine (quadrivalent), NOV-002, octreotide, lanreotide, ancestirn, agalsidase beta, agalsidase alfa, laronidase, prezatide copper acetate (topical gel), rasburicase, ranibizumab, Actimmune, PEG-Intron, Tricomin, recombinant house dust mite allergy desensitization injection, recombinant human parathyroid hormone (PTH) 1-84 (sc, osteoporosis), epoetin delta, transgenic antithrombin III, Granditropin, Vitrase, recombinant insulin, interferon-alpha (oral lozenge), GEM-21S, vapreotide, idursulfase, omnapatrilat, recombinant serurn albumin, certolizumab pegol, glucarpidase, human recombinant C1 esterase inhibitor (angioedema), lanoteplase, recombinant human growth hormone, enfuvirtide (needle-free injection, Biojector 2000), VGV-1, interferon (alpha), lucinactant, aviptadil (inhaled, pulmonary disease), icatibant, ecallantide, omiganan, Aurograb, pexigananacetate, ADI-PEG-20, LDI-200, degarelix, cintredelinbesudotox, Favld, MDX-1379, ISAtx-247, liraglutide, teriparatide (osteoporosis), tifacogin, AA4500, T4N5 liposome lotion, catumaxomab, DWP413, ART-123, Chrysalin, desmoteplase, amediplase, corifollitropinalpha, TH-9507, teduglutide, Diamyd, DWP-412, growth hormone (sustained release injection), recombinant G-CSF, insulin (inhaled, AIR), insulin (inhaled, Technosphere), insulin (inhaled, AERx), RGN-303, DiaPep277, interferon beta (hepatitis C viral infection (HCV)), interferon alfa-n3 (oral), belatacept, transdermal insulin patches, AMG-531, MBP-8298, Xerecept, opebacan, AIDSVAX, GV-1001, LymphoScan, ranpirnase, Lipoxysan, lusupultide, MP52 (beta-tricalciumphosphate carrier, bone regeneration), melanoma vaccine, sipuleucel-T, CTP-37, Insegia, vitespen, human thrombin (frozen, surgical bleeding), thrombin, TransMID, alfimeprase, Puricase, terlipressin (intravenous, hepatorenal syndrome), EUR-1008M, recombinant FGF-I (injectable, vascular disease), BDM-E, rotigaptide, ETC-216, P-113, MBI-594AN, duramycin (inhaled, cystic fibrosis), SCV-07, OPI-45, Endostatin, Angiostatin, ABT-510, Bowman Birk Inhibitor Concentrate, XMP-629, 99 mTc-Hynic-Annexin V, kahalalide F, CTCE-9908, teverelix (extended release), ozarelix, rornidepsin, BAY-504798, interleukin4, PRX-321, Pepscan, iboctadekin, rhlactoferrin, TRU-015, IL-21, ATN-161, cilengitide, Albuferon, Biphasix, IRX-2, omega interferon, PCK-3145, CAP-232, pasireotide, huN901-DMI, ovarian cancer immunotherapeutic vaccine, SB-249553, Oncovax-CL, OncoVax-P, BLP-25, CerVax-16, multi-epitope peptide melanoma vaccine (MART-1, gp100, tyrosinase), nemifitide, rAAT (inhaled), rAAT (dermatological), CGRP (inhaled, asthma), pegsunercept, thymosinbeta4, plitidepsin, GTP-200, ramoplanin, GRASPA, OBI-1, AC-100, salmon calcitonin (oral, eligen), calcitonin (oral, osteoporosis), examorelin, capromorelin, Cardeva, velafermin, 131I-TM-601, KK-220, T-10, ularitide, depelestat, hematide, Chrysalin (topical), rNAPc2, recombinant Factor V111 (PEGylated liposomal), bFGF, PEGylated recombinant staphylokinase variant, V-10153, SonoLysis Prolyse, NeuroVax, CZEN-002, islet cell neogenesis therapy, rGLP-1, BIM-51077, LY-548806, exenatide (controlled release, Medisorb), AVE-0010, GA-GCB, avorelin, AOD-9604, linaclotid eacetate, CETi-1, Hemospan, VAL (injectable), fast-acting insulin (injectable, Viadel), intranasal insulin, insulin (inhaled), insulin (oral, eligen), recombinant methionyl human leptin, pitrakinra subcutancous injection, eczema), pitrakinra (inhaled dry powder, asthma), Multikine, RG-1068, MM-093, NBI-6024, AT-001, PI-0824, Org-39141, Cpn10(autoimmune iseases/inflammation), talactoferrin (topical), rEV-131 (ophthalmic), rEV-131 (respiratory disease), oral recombinant human insulin (diabetes), RPI-78M, oprelvekin (oral), CYT-99007 CTLA4-Ig, DTY-001, valategrast, interferon alfa-n3 (topical), IRX-3, RDP-58, Tauferon, bile salt stimulated lipase, Merispase, alaline phosphatase, EP-2104R, Melanotan-II, bremelanotide, ATL-104, recombinant human microplasmin, AX-200, SEMAX, ACV-1, Xen-2174, CJC-1008, dynorphin A, SI-6603, LAB GHRH, AER-002, BGC-728, malaria vaccine (virosomes, PeviPRO), ALTU-135, parvovirus B19 vaccine, influenza vaccine (recombinant neuraminidase), malaria/HBV vaccine, anthrax vaccine, Vacc-5q, Vacc-4x, HIV vaccine (oral), HPV vaccine, Tat Toxoid, YSPSL, CHS-13340, PTH(1-34) liposomal cream (Novasome), Ostabolin-C, PTH analog (topical, psoriasis), MBRI-93.02, MTB72F vaccine (tuberculosis), MVA-Ag85A vaccine (tuberculosis), FARA04, BA-210, recombinant plague F1V vaccine, AG-702, OxSODrol, rBetV1, Der-p1/Der-p2/Der-p7 allergen-targeting vaccine (dust mite allergy), PR1 peptide antigen (leukemia), mutant ras vaccine, HPV-16 E7 lipopeptide vaccine, labyrinthin vaccine (adenocarcinoma), CML vaccine, WT1-peptide vaccine (cancer), IDD-5, CDX-110, Pentrys, Norelin, CytoFab, P-9808, VT-111, icrocaptide, telbermin (dermatological, diabetic foot ulcer), rupintrivir, reticulose, rGRF, P1A, alpha-galactosidase A, ACE-011, ALTU-140, CGX-1160, angiotensin therapeutic vaccine, D-4F, ETC-642, APP-018, rhMBL, SCV-07 (oral, tuberculosis), DRF-7295, ABT-828, ErbB2-specific immunotoxin (anticancer), DT3SSIL-3, TST-10088, PRO-1762, Combotox, cholecystokinin-B/gastrin-receptor binding peptides, 111In-hEGF, AE-37, trasnizumab-DM1, Antagonist G, IL-12 (recombinant), PM-02734, IMP-321, rhIGF-BP3, BLX-883, CUV-1647 (topical), L-19 based radioimmunotherapeutics (cancer), Re-188-P-2045, AMG-386, DC/1540/KLH vaccine (cancer), VX-001, AVE-9633, AC-9301, NY-ESO-1 vaccine (peptides), NA17.A2 peptides, melanoma vaccine (pulsed antigen therapeutic), prostate cancer vaccine, CBP-501, recombinant human lactoferrin (dry eye), FX-06, AP-214, WAP-8294A (injectable), ACP-HIP, SUN-11031, peptide YY [3-36] (obesity, intranasal), FGLL, atacicept, BR3-Fc, BN-003, BA-058, human parathyroid hormone 1-34 (nasal, osteoporosis), F-18-CCR1, AT-1100 (celiac disease/diabetes), JPD-003, PTH(7-34) liposomal cream (Novasome), duramycin (ophthalmic, dry eye), CAB-2, CTCE-0214, GlycoPEGylated erythropoietin, EPO-Fc, CNTO-528, AMG-114, JR-013, Factor XIII, aminocandin, PN-951, 716155, SUN-E7001, TH-0318, BAY-73-7977, teverelix (immediate release), EP-51216, hGH (controlled release, Biosphere), OGP-I, sifuvirtide, TV4710, ALG-889, Org-41259, rhCC10, F-991, thymopentin (pulmonary diseases), r(m)CRP, hepatoselective insulin, subalin, L19-IL-2 fusion protein, elafin, NMK-150, ALTU-139, EN-122004, rhTPO, thrombopoietin receptor agonist (thrombocytopenic disorders), AL-108, AL-208, nerve growth factor antagonists (pain), SLV-317, CGX-1007, INNO-105, oral teriparatide (eligen), GEM-OS1, AC-162352, PRX-302, LFn-p24 fusion vaccine (Therapore), EP-1043, S pneumoniae pediatric vaccine, malaria vaccine, Neisseria meningitidis Group B vaccine, neonatal group B streptococcal vaccine, anthrax vaccine, HCV vaccine (gpE1+gpE2+MF-59), otitis media therapy, HCV vaccine (core antigen+ISCOMATRIX), hPTH(1-34) (transdermal, ViaDerm), 768974, SYN-101, PGN-0052, aviscumnine, BIM-23190, tuberculosis vaccine, multi-epitope tyrosinase peptide, cancer vaccine, enkastim, APC-8024, GI-5005, ACC-001, TTS-CD3, vascular-targeted TNF (solid tumors), desmopressin (buccal controlled-release), onercept, and TP-9201.

In certain embodiments, the heterologously produced protein is an enzyme or biologically active fragments thereof. Suitable enzymes include but are not limited to: oxidoreductases, transferases, hydrolases, lyases, isomerases, and ligases. In certain embodiments, the heterologously produced protein is an enzyme of Enzyme Commission (EC) class 1, for example an enzyme from any of EC 1.1 through 1.21, or 1.97. The enzyme can also be an enzyme from EC class 2, 3, 4, 5, or 6. For example, the enzyme can be selected from any of EC 2.1 through 2.9, EC 3.1 to 3.13, EC 4.1 to 4.6, EC 4.99, EC 5.1 to 5.11, EC 5.99, or EC 6.1-6.6.

In certain embodiments the heterologously produced protein is an acetylase, acylase, aldolase, amidase, amylase, ATPase, carboxylase, cyclase, cycloisomerase, deacetylase, deacylase, decarboxylase, decyclase, dehalogenase, dehydratase, dehydrogenase, dehydroxylase, demethylase, depolymerase, desaturase, dioxygenase, dismutase, endonuclease, epimerase, epoxidase, esterase, exonuclease, galactosidase, glucosidase, glycosidase, glycosylase, halogenase, hydratase, hydrogenase, hydrolase, hydroxylase, hydroxytransferase, isomerase, ligase, lipase, lipoxygenase, lyase, methylesterase, monooxygenase, mutase, nuclease, nucleosidase, nucleotidase, oxidase, oxidoreductase, oxygenase, peptidase, peroxidase, phosphatase, phosphodiesterase, phospholipase, polymerase, polymerase, protease, proteinase, racemase, reductase, reductoisomerase, rionuclease, ribonuclease, synthase, synthetase, tautomerase, thioesterase, thioglucosidase, thiolesterase, topoisomerase, or transhydrogenase. Suitable kinases include but are not limited to: tyrosine kinases, serine kinases, threonine kinases, aspartine kinases, and histidine kinases. Suitable phosphorylases include but are not limited to: tyrosine phosphorylases, serine phosphorylases, and threonine phosphorylases.

In certain embodiments, the heterologously produced protein is an isomerase or biologically active fragments thereof. Suitable isomerases include but are not limited to: isopentenyl diphosphate (“IPP”) isomerase or biologically active fragments thereof. In certain embodiments, the heterologously produced protein is a synthase or biologically active fragments thereof. Suitable synthases include but are not limited to: prenyl diphosphate synthases and terpene synthases. Suitable prenyl diphosphate synthases, or prenyltransferases, for example, the prenyltransferase can be an E-isoprenyl diphosphate synthase, including, but not limited to, geranyl diphosphate (GPP) synthase, farnesyl I diphosphate (FPP) synthase, geranylgeranyl diphosphate (GGPP) synthase, hexaprenyl diphosphate (HexPP) synthase, heptaprenyl diphosphate (HepPP) synthase, octaprenyl (OPP) diphosphate synthase, solanesyl diphosphate (SPP) synthase, decaprenyl diphosphate (DPP) synthase, chicle synthase, and gutta-percha synthase; and a Zisoprenyl diphosphate synthase, including, but not limited to, nonaprenyl diphosphate (NPP) synthase, undecaprenyl diphosphate (UPP) synthase, dehydrodolichyl diphosphate synthase, eicosaprenyl diphosphate synthase, natural rubber synthase, and other Zisoprenyl diphosphate syntheses. In some embodiments, the prenyltransferase is encoded by an exogenous sequence.

The nucleotide sequences of numerous prenyl transferases from a variety of species are known, and can be used or modified for use in generating heterologous sequences for producing the aforementioned heterologous proteins. For example, sequences for the following are publicly available: human farnesyl pyrophosphate synthetase InRNA (GenBank Accession No. J05262; Homo sapiens); farnesyl diphosphate synthetase (FPP) gene (GenBank Accession No. J05091; Saccharomyces cerevisiae); isopentenyl diphosphate:dimethylallyl diphosphate isomerase gene (J05090; Saccharomyces cerevisiae); Wang and Ohnuma (2000) Biochim. Biophys. Acta 1529:33-48; U.S. Pat. No. 6,645,747; Arabidopsis thaliana farnesyl pyrophosphate synthetase 2 (FPS2)/FPP synthetase 2/farnesyl diphosphate synthase 2 (At4 g17190) mRNA (GenEBank Accession No. NM_{—}202836); Ginkgo biloba geranylgeranyl diphosphate synthase (ggpps) mRNA (GenBank Accession No. AY371321); Arabidopsis thaliana geranylgeranyl pyrophosphate synthase (GGPS1)/GGPP synthetase /farnesyltranstansferase (At4g36810) mRNA (GenBank Accession No. NM_{—}119845); Synechococcus elongatus gene for farnesyl, geranylgeranyl, geranylfarnesyl, hexaprenyl, heptaprenyl diphosphate synthase (SeIF-HepPS) (GenBank Accession No. AB016095).

In other embodiments, the produced protein is a terpene synthase, including but not limited to: amorpha-4,11-iene synthase, β-caryophyllene synthase, germacrene A synthase, 8-epicedrol synthase, valencene synthase, (+)-δ-cadinene synthase, germacrene C synthase, (E)-β-farnesene synthase, casbene synthase, vetispiradiene synthase, 5-epi-aristolochene synthase, aristoichene synthase, α-humulene synthase, (E,E)-α-farnesene synthase, (−)-β-pinene synthase, γ-terpinene synthase, limonene cyclase, linalool synthase, 1,8-cineole synthase, (+)-sabinene synthase, E-α-bisabolene synthase, (+)-bornyl diphosphate synthase, levopimaradiene synthase, abietadiene synthase, isopimaradiene synthase, (E)-γ-bisabolene synthase, taxadiene synthase, copalyl pyrophosphate synthase, kaurene synthase, longifolene synthase, γ-humulene synthase, δ-selinene synthase, β-phellandrene synthase, limonene synthase, myrcene synthase, terpinolene synthase, (−)-campbene synthase, (+)-3-carene synthase, syn-copalyl diphosphate synthase, α-terpineol synthase, syn-pimara-7,15-diene synthase, ent-sandaaracopimiaradiene synthase, stemer-13-ene synthase, E-β-ocimene, S-linalool synthase, geraniol synthase, γ-terpinene synthase, linalool synthasel, E-β-ocimene synthase, epi-cedrol synthase, α-zingiberene synthase, guaiadiene synthase, cascarilladiene synthase, cis-muuroladiene synthase, aphidicolan-16b-ol synthase, elizabethatriene synthase, sandalol synthase, patchoulol synthase, zinzanol synthase, cedrol synthase, scareol synthase, copalol synthase, and manool synthase.

In some embodiments, the heterologously produced protein is an enzyme, or biologically active fragments thereof, that functions in a metabolic pathway. The heterologously produced protein may be an enzyme that functions in a catabolic pathway. Suitable examples of catabolic pathways include but are not limited to pathways of aerobic respiration, which include glycolysis, oxidative decarboxylation of pyruvate, citric acid cycle, and oxidative phosphorylation; and pathways of anaerobic respiration (fermentation). In other embodiments, the heterologously produced protein is an enzyme that functions in an anabolic pathway. Suitable examples of anabolic pathways include but are not limited to the mevalonate-dependent (“MEV”) pathway and the mevalonate-independent (“DXP”) pathway for the production of isopentenyl diphosphate isomerase (“IPP”). IPP can be further converted to isoprenoids For example, heterologous sequences encoding the MEV pathway enzymes that play a role in controlling the metabolic flux of the pathway, such as those involved in rate limiting steps, or involved in the synthesis of metabolic intermediates may be used in the present invention. Exemplary MEV pathway enzymes of this category include but are not linited to HMG-CoA reductase, HMG-CoA synthase, and mevalonate kinase.

Enzymes, or biologically active fragments thereof, involved in the DXP pathway have been identified and isolated and may be used. These enzymes include 1-deoxyxylulose-5-phosphate synthase (encoded by the “dxs” gene), 1-deoxyxylulose-5-phosphate reductoisomerase (encoded by the “dxr” gene, also known the “ispC” gene), 2C-methyl-D-erythritol cytidyltraisferase enzyme (encoded by the “ispD” gene, also known as the “ygbP” gene), 4-diphosphocytidyl-2-C-methylerythritol kinase (encoded by the “ispE” gene, also known the “ychB” gene), 2C-methyl-D-erythritol 2,4-cyclodiphosphate synthase (encoded by the “ispF” gene, also known as the “ygbB” gene), CTP synthase (encoded by the “pyrG” gene, also known as the “ispF” gene), an enzyme involved in the formation of dimethylallyl diphosphate (encoded by the “lytb” gene, also known as the “ispH” gene), an enzyme involved in the synthesis of 1-hydroxy-2-methyl-2-(E)-butenyl 4-diphosphate synthase (encoded by the “gepE” gene, also known as the “ispG” gene).

Exemplary polypeptide/nucleotide sequences of the DXP pathway include but are not limited to D-1-deoxyxylulose 5-phosphate synthase (Escherichia coli, ACCESSION# AF035440), 1-deoxy-D-xylulose-5-phosphate synthase (Pseudomonas putida KT2440, ACCESSION# NC_{—}002947 locus_tag PP0527), 1-deoxyxylulose-5-phosphate synthase (Salmonella enterica subsp. enterica serovar Paratyphi A str. ATCC 9150, ACCESSION# CP000026, locus tag SPA2301), 1-deoxy-D-xylulose-5-phosphate synthase (Rhodobacter sphaeroides 2.4.1, ACCESSION# NC_{—}007493 locus_tag RSP_{—}0254), 1-deoxy-D-xylulose-5-phosphate synthase (Rhodopseudomonas palustris CGA009, ACCESSION# NC_{—}005296 locus_tag RPA0952), 1-deoxy-D-xylulose-5-phosphate synthase (Xylella fastidiosa Temecula1, ACCESSION# NC_{—}004556 locus_tag PD1293), 1-deoxy-D-xylulose-5-phosphate synthase (Arabidopsis thaliana, ACCESSION# NC_{—}003076 locus_tag AT5G11380), 1-deoxy-D-xylulose 5-phosphate reductoisomerase (Escherichia coli, ACCESSION# AB013300), 1-deoxy-D-xylulose 5-phosphate reductoisomerase (Arabidopsis thaliana, ACCESSION# AF148852), 1-deoxy-D-xylulose 5-phosphate reductoisomerase (Pseudomonas putida KT2440, ACCESSION# NC_{—}002947 locus_tag PF1597), 1-deoxy-D-xylulose 5-phosphate reductoisomerase (Streptomyces coelicolor A3(2), ACCESSION# AL939124 Locus_tag CO5694), 1-deoxy-D-xylulose 5-phosphate reductoisomerase (Rhodobacter sphaeroides 2.4.1, ACCESSION# NC_{—}007493 locus_tag RSP_{—}2709), 1-deoxy-D-xylulose 5-phosphate reductoisomerase (Pseudomonas fluorescens PfO-1, ACCESSION# NC_{—}007492 locus_tag Pfl_{—}1107), 4-diphosphocytidyl-2C-methyl-D-erythritol synthase (Escherichia coli, ACCESSION# AF230736), 4-diphosphocytidyl-2-methyl-D-erithritol synthase (Rhodobacter sphaeroides 2.4.1, ACCESSION#, NC_{—}007493 locus_tag, RSP_{—}2835), 4-Diphosphocytidyl-2C-methyl-D-erydritol synthase (Arabidopsis thaliana, ACCESSION# NC_{—}003071 locus_tag AT2G02500), 2-C-methyl-D-erythritol 4-phosphate cytidylyltransferase (Pseudomonas putida KT2440, ACCESSION# NC_{—}002947 locus_tag PP1614), 4-diphosphocytidyl-2C-methyl-D-erythritol kinase(ispE) gene (Escherichia coli, ACCESSION# AF216300), 4-diphosphocytidyl-2C-methyl-D-erythritol kinase (ispE) (Rhodobacter sphaeroides 2.4.1, ACCESSION# NC_{—}007493 locus_tag RSP_{—}1779), 2C-methyl-D-erythritol 2,4-cyclodiphosphate synthase (Escherichia coli, ACCESSION# AF230738), 2C-methyl-D-erythritol 2,4-cyclodiphosphate synthase (Rhodobacter sphaeroides 2.4.1, ACCESSION# NC_{—}007493 locus_tag RSP_{—}6071), 2-C-methyl-D-erythritol 2,4-cyclodiphosphate synthase (Pseudomonas putida KT2440, ACCESSION# NC_{—}002947 locus_tag PP1618), 1-hydroxy-2-methyl-2-(E)-butenyl 4-diphosphate synthase (Escherichia coli, ACCESSION# AY033515), 4-hydroxy-3-methylbut-2-en-1-yl diphosphate synthase (Pseudomonas putida KT2440, ACCESSION# NC_{—}002947 locus_tag PP0853), 4-hydroxy-3-methylbut-2-en-1-yl diphosphate synthase (Rhodobacter sphaeroides 2.4.1, ACCESSION# NC_{—}007493 locus_tag RSP_{—}2982), IspH (LytB) (Escherichia coli, ACCESSION# AY062212), 4-hydroxy-3-methylbut-2-enyl diphosphate reductase (Pseudomonas putida KT2440, ACCESSION# NC_{—}002947 locus_tag PP0606), and any other DXP pathway genes disclosed in US Application 20060121558, which is incorporated herein by reference.

Nucleotide sequences encoding enzymes involved in the reverse TCA cycle are also known in the art and may be used as heterologous sequences to produce heterologous products that are enzymes in the reverse TAC cycle. Exemplary polypeptide/nucleotide sequences of the TCA Cycle include but are not limited to 2-oxoglutarate ferredoxin oxidoreductase (Hydrogenobacter thermophilus, ACCESSION# AB046568, Bordetella bronchiseptica, ACCESSION# Y10540), (Escherichia coli, ACCESSION# U09868), fumarate reductase (Mannheimia haemolytica, ACCESSION# DQ680277, Escherichia coli, ACCESSION# AY692474), pyruvate:ferredoxin oxidoreductase (Hydrogenobacter thermophilus, ACCESSION# AB042412), isocitrate dehydrogenase (Chlorobium limicola, ACCESSION# AB076021, Rattus norvegicus, ACCESSION# NM_{—}031551), ATP-citrate synthase (Chlorobium limicola, ACCESSION# AB054670, Saccharomyces cerevisiae, ACCESSION# X00782), phosphoenolpyruvate synthase (Escherichia coli, ACCESSION# X59381, M69116), phosphoenolpyruvate carboxylase (Streptococcus thermophilus, ACCESSION# AM 167938, Lupinus luteus, ACCESSION# AM235211), malate dehydrogenase (Chlorobaculum tepidum, ACCESSION# X80838, Mus musculus, ACCESSION# X07297, Klebsiella pneumoniae, ACCESSION# AM051137), and/or fumarase (Rhizopus oryzae, ACCESSION# X78576, Solanum tuberosum, ACCESSION# X91615). Any of these reverse TCA cycle nucleic acids can be used to generate an isoprenoid-producing recombinant host cell according to the methods of this invention.

A wide selection of nucleotide sequences encoding MEV pathway enzymes is available in the art and the enzymes or biologically active fragments thereof can readily be employed in constructing the subject heterologous sequences. The following are non-limiting examples of known nucleotide sequences encoding MEV pathway gene products, with GenBalnk Accession numbers and organism of origin following each MEV pathway enzyme, in parentheses: acetoacetyl-CoA thiolase: (NC_{—}000913 REGION: 2324131 . . . 2325315; E. coli), D49362; Paracoccus denitrificans), and (L20428; Saccharomyces cerevisiae); HMGS: (NC_{—}001145. complement 19061.20536; Saccharomyces cerevisiae), (X96617; Saccharomyces cerevisiae), (X83882; Arabidopsis thaliana), (AB037907; Kitasatospora griseola), and (BT007302; Homo sapiens) (NC_{—}002758, Locus tag SAV2546, GeneID 1122571; Staphylococcus aureus); HMGR: (NM_{—}206548; Drosophila melanogaster), (NGC002758, Locus tag SAV2545, GeneID 1122570; Staphylococcus aureus), (NM204485; Gallus gallus), (AB015627; Streptomyces sp. KO-3988), (AF542543; Nicotiana attenuata), (AB037907; Kitasatospora griseola), (AX128213, providing the sequence encoding a truncated HMGR; Saccharomyces cerevisiae), and (NC_{—}001145: complement (115734 . . . 118898; Saccharomyces cerevisiae)); MK: (L77688; Arabidopsis thaliana), and (X55875; Saccharomyces cerevisiae); PMK: (AF429385; Hevea brasiliensis), (NM_{—}006556; Homo sapiens), (NC_{—}001145. complement 712315.713670; Saccharomyces cerevisiae); MPD: (X597557; Saccharomyces cerevisiae), (AF290095; Enterococcus faecium), and (U49260; Homo sapiens); and IDI: (NC_{—}000913, 3031087 . . . 3031635; E. coli), and (AF082326; Haematococcus pluvialis).

The products of the metabolic pathways may include hydrocarbons, and derivatives there of. For example, saturated, unsaturated, cycloalkanes, and aromatic hydrocarbons may be produced by the methods of the present invention. For example, terpenes and terpenoids, such as isoprenoids, may be produced as a result of the production of heterologous proteins such as an enzyme of the MEV pathway that was encoded by a heterologous sequence of the present invention.

Isoprenoids, including, without limitation, any C₅ through C₂₀ or higher carbon number isoprenoids, may be a heterologous product produced by the methods described herein. The following describes, without limitation, exemplary isoprenoids, such as any C₅ through C₂₀ or higher carbon number isoprenoids. Examples of C₅ compounds of the invention may be derived from IPP or DMAPP. These compounds are also known as hemiterpenes because they are derived from a single isoprene unit (IPP or DMAPP). Isoprene, whose structure is

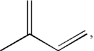

is found in many plants. Isoprene is typically made from IPP by isoprene synthase. Illustrative examples of suitable nucleotide sequences include but are not limited to: (AB198190; Populus alba) and (AJ294819; Polulus alba×Polulus tremula) and may be the heterologous sequence of used in the present invention.

C₁₀ compounds, also known as monoterpenes because they are derived from two isoprene units, of the present invention may be derived from geranyl pyrophosphate (GPP) which is made by the condensation of IPP with DMAPP. In certain embodiments, the host cells of the present invention comprises a heterologous sequence that encodes an enzyme that converts IPP and DMAPP into GPP. An enzyme known to catalyze this step is, for example, geranyl pyrophosphate synthase. Illustrative examples of nucleotide sequences for geranyl pyrophosphate synthase include but are not limited to: (AF513111; Abies grandis), (AF513112; Abies grandis), (AF513113; Abies grandis), (AY534686; Antirrhinum majus), (AY534687; Antirrhinum majus), (Y17376; Arabidopsis thaliana), (AE016877, Locus AP11092; Bacilus cereus; ATCC 14579), (AJ243739; Citrus sinensis), (AY534745; Clarkia breweri), (AY953508; Ips pini), (DQ286930; Lycopersicon esculentum), (AF182828; Mentha×piperita), (AF182827; Mentha×piperita), (MP1249453; Mentha×piperita), (PZE431697, Locus CAD24425; Paracoccus zeaxanthinifaciens), (AY866498; Picrorhiza kurrooa), (AY351862; Vitis vinifera), and (AF203881, Locus AAF12843; Zymomonas mobilis). GPP can then be subsequently converted to a variety of C₁₀ compounds. Illustrative examples of C₁₀ compounds include but are not limited to following monoterpenes.

For example, the monoterpene may be carene, whose structure is

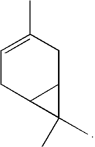

Carene is typically made from GPP by carene synthase. Illustrative examples of suitable nucleotide sequences include but are not limited to: (AF461460, REGION 43 . . . 1926; Picea abies) and (AF527416, REGION: 78 . . . 1871; Salvia stenophylla) for use as heterologous sequences that encode carene synthase.

Another monoterpene, such as geraniol, (also known as rhodnol), whose structure is

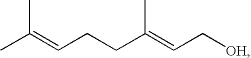

may be a product produced by the present invention. Geraniol is typically made from OPP by geraniol synthase. Illustrative examples of suitable nucleotide sequences include but are not limited to: (AJ457070; Cinnamomum tenuipilum), (AY362553; Ocimum basilicum), (DQ234300; Perilla frutescens strain 1864), (DQ234299; Perilla citriodora strain 1861), (DQ234298; Perilla citriodora strain 4935), and (DQ088667; Perilla citriodora) for encoding geraniol synthase that may be used a a heterologous sequence of the present invention.

The monoterpene, linalool, whose structure is

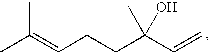

is typically made from GPP by linalool synthase and may be produced by the present invention. Illustrative examples of a suitable nucleotide sequence include, but are not limited to: (AF497485; Arabidopsis thaliana), (AC002294, Locus AAB71482; Arabidopsis thaliana), (AY059757; Arabidopsis thaliana), (NM_{—}104793; Arabidopsis thaliana), (AF154124; Artemisia annua), (AF067603; Clarkia breweri), (AF067602; Clarkia concinna), (AF067601; Clarkia breweri), (U58314; Clarkia breweri), (AY840091; Lycopersicon esculentum), (DQ263741; Lavandula angustifolia), (AY083653; Mentha citrate), (AY693647; Ocimum basilicum), (XM_{—}463918; Oryza sativa), (AP004078, Locus BAD07605; Oryza sativa), (XM_{—}463918, Locus XP_{—}463918; Oryza sativa), (AY917193; Perilla citriodora), (AF271259; Perilla frutescens), (AY473623; Picea abies), (DQ195274; Picea sitchensis), and (AF444798; Perilla frutescens var. crispa cultivar No. 79). These sequences may be used as heterologous sequences of the present invention.

Another monoterpene, limonene whose structure is

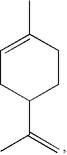

is typically made from GPP by limonene synthase. Illustrative examples of suitable nucleotide sequences that may be used as heterologous sequences of the present invention include but are not limited to: (+)-limonene synthases (AF514287, REGION: 47 . . . 1867; Citrus limon) and (AY055214, REGION: 48 . . . 1889; Agastache rugosa) and (−)-limonene synthases (DQ195275, REGION: 1 . . . 1905; Picea sitchensis), (AF006193, REGION: 73.1986; Abies grandis), and (MC4SLSP, REGION: 29 . . . 1828; Mentha spicata).

The monoterpene, myrcene, whose structure is

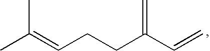

is typically made from GPP by myrcene synthase and is another product that may be produced by the present invention. Illustrative examples of suitable nucleotide sequences that may be used as heterologous sequences of the present invention include but are not limited to: (187908; Abies grandis), (AY195609; Antirrhinum majus), (AY195608; Antirrhinum majus), (NM_{—}127982; Arabidopsis thaliana TPS10), NM_{—}113485; Arabidopsis thaliana ATTPS-CIN), (NM_{—}13483; Arabidopsis thaliana ATIPS-CIN), (AF271259; Perilla frutescens), (AY473626; Picea abies), (AF369919; Picea abies), and (AJ304839; Quercus ilex).

Another monoterpene, ocimene, α- and β-Ocimene, whose structures are

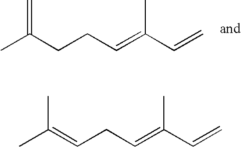

respectively, are typically made from GPP by ocimene synthase, a synthase that may be encoded by the heterologous sequences of the present invention. Illustrative examples of suitable nucleotide sequences that may be used as heterologous sequences include but are not limited to: (AY195607; Antirrhinum majus), (AY195609; Antirrhinum majus), (AY195608; Antirrhinum majus), (AK221024; Arabidopsis thaliana), (NM_{—}113485; Arabidopsis thaliana ATTPS-CIN), (NM_{—}113483; Arabidopsis thaliana ATTPS-CIN), (NM_{—}117775; Arabidopsis thaliana ATTPS03), (NM_{—}001036574; Arabidopsis thaliana ATTPS03), (NM_{—}127982; Arabidopsis thaliana TPS10), (AB110642; Citrus unshiu CitMTSL4), and (AY575970; Lotus corniculatus var. japonicus).

Another monoterpene, α-pinene whose structure is

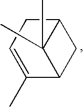

is typically made from GPP by α-pinene synthase, a synthase that may be encoded by the heterologous sequences of the present invention. Illustrative examples of suitable nucleotide sequences that may be used as heterologous sequences to encode the synthase include but are not limited to: (+) α-pinene synthase (AF543530, REGION: 1 . . . 1887; Pinus taeda), (−)α-pinene synthase (AF543527, REGION: 32 . . . 1921; Pinus taeda), and (+)/(−)α-pinene synthase (AGU87909, REGION: 6111892; Abies grandis).

Another monoterpene, β-pinene, whose structure is

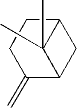

is typically made from GPP by β-pinene synthase. a synthase that may be encoded by the heterologous sequences of the present invention. Illustrative examples of suitable nucleotide sequences that may be used as heterologous sequences to encode the synthase include but are not limited to: (−) β-pinene synthases (AF276072, REGION: 1 . . . 1749; Artemisia annua) and (AF514288, REGION: 26 . . . 1834; Citrus limon).

Another monoterpene, sabinene, whose structure is

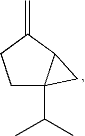

is typically made from GPP by sabinene synthase, a synthase that may be encoded by the heterologous sequences of the present invention. An illustrative example of a suitable nucleotide sequence that may be used as a heterologous sequence of include but is not limited to AF051901, REGION: 26 . . . 1798 from Salvia officinalis.

Another monoterpene, γ-terpinene, whose structure is

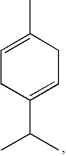

is typically made from GPP by a γ-terpinene synthase, a synthase that may be encoded by the heterologous sequences of the present invention. Illustrative examples of suitable nucleotide sequences that may be used as heterologous sequences include but are not limited to: (AF514286, REGION: 30 . . . 1832 from Citrus limon) and (AB110640, REGION 1 . . . 1803 from Citrus unshiu).

Another monoterpene, terpinolene, whose structure is

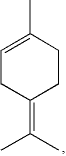

is typically made from GPP by terpinolene synthase, a synthase that may be encoded by the heterologous sequences of the present invention. Illustrative examples of suitable nucleotide sequences that may be used as heterologous sequences include but are not limited to: (AY693650 from Oscimum basilicum) and (AY906866, REGION: 10 . . . 1887 from Pseudotsuga menziesii).

Heterologous products of the present invention may also be C₁₅ compounds. The C₁₅ compounds are generally derive from farnesyl pyrophosphate (FPP) which is made by the condensation of two molecules of IPP with one molecule of DMAPP. An enzyme known to catalyze this step is, for example, farnesyl pyrophosphate synthase. These C₁₅ compounds are also known as sesquiterpenes because they are derived from three isoprene units. In certain embodiments, the host cells of the present invention comprises a heterologous sequence that encodes an enzyme that converts IPP and DMAPP into FPP.

Illustrative examples of nucleotide sequences which encode farnesyl pyrophosphate that may be heterologous sequences of the present invention include but are not limited to: (AF461050; Bos taurus), (AB003187, Micrococcus luteus), (AE009951, Locus AAL95523; Fusobacterium nucleatum subsp. nucleatum ATCC 25586), (GFFPPSGEN; Gibberella fujikurio), (AB016094, Synechococcus elongatus), (CP000009, Locus AAW60034; Gluconobacter oxydans 621H), (AF019892; Helianthus annuus), (HUMFAPS; Homo sapiens), (KLPFPSQCR; Kluyveromyces lactis), (LAU15777; Lupinus albus), (LAU20771; Lupinus albus), (AF309508; Mus musculus), (NCFPPSGEN; Neurospora crassa), (PAFPS1; Parthenium argentatum), (PAFPS2; Parthenium argentatum), (RATFAPS; Rattus norvegicus), (YSCFPP; Saccharomyces cerevisiae), D89104; Schizosaccharomyces pombe), (CP000003, Locus AAT87386; Streptococcus pyogenes), (CP000017, Locus AAZ51849; Streptococcus pyogenes), (CN008022, Locus YP 598856; Streptococcus pyogenes MGAS10270), (NC_{—}008023, Locus YP_{—}600845; Streptococcus pyogenes MGAS2096), (NC_{—}008024, Locus YP_{—}602832; Streptococcus pyogenes MGAS10750), and (MZEFPS; Zea mays, (AB021747, Oryza sativa FPPS1 gene for farnesyl diphosphate synthase), (AB028044, Rhodobacter sphaeroides), (AB028046, Rhodobacter capsulatus), (AB028047, Rhodovulum sulfldophium), (AAU36376; Artemisia annua), (AF112881 and AF136602, Artemisia annua), (AF384040, Mentha×piperita), (D00694, Escherichia coli K-12), (D13293, B. stearothermophilus), (D85317, Oryza sativa), (ATU80605; Arabidopsis thaliana), (ATIFPS2R; Arabidopsis thaliana), (X75789, A. thaliana), (Y12072, G. arboreum), (Z49786, H. brasiliensis), (U80605, Arabidopsis thaliana farnesyl diphosphate synthase precursor (FPS1) mRNA, complete cds), (X76026, K. lactis FPS gene for farnesyl diphosphate synthetase, QCR8 gene for bcl complex, subunit VIII), (X82542, P. argentatum mRNA for farnesyl diphosphate synthase (FPS1), (X82543, P. argentatum mRNA for farnesyl diphosphate synthase (FPS2), (BC010004, Homo sapiens, farnesyl diphosphate synthase (farnesyl pyrophosphate synthetase, dimethylallyltranstransferase, geranyltranstransferase), clone MGC 15352 IMAGE, 4132071, mRNA, complete cds) (AF234168, Dictyostelium discoideum farnesyl diphosphate synthase (Dfps), (L46349, Arabidopsis thaliana farnesyl diphosphate synthase (FPS2) mRNA, complete cds), (L46350, Arabidopsis thaliana farnesyl diphosphate synthase (FPS2) gene, complete cds), (L46367, Arabidopsis thaliana farnesyl diphosphate synthase (FPS1) gene, alternative products, complete cds), (M89945, Rat farnesyl diphosphate synthase gene, exons 1-8), (NM_{—}002004, Homo sapiens farnesyl diphosphate synthase (farnesyl pyrophosphate synthetase, dimethylallyltranstransferase-, geranyltranstransferase) (FDPS), mRNA), (1536376, Artemisia annua farnesyl diphosphate synthase (fps1) mRNA, complete cds), (XM_{—}001352, Homo sapiens farnesyl diphosphate synthase (farnesyl pyrophosphate synthetase, dimethylallyltranstransferase-, geranyltranstransferase) (FOPS), MRINA), (XM_{—}034497, Homo sapiens farnesyl diphosphate synthase (farnesyl pyrophosphate synthetase, dimethylallyltranstransferase, geranyltranstransferase) (FDPS), mRNA), (XM_{—}034498, Homo sapiens farnesyl diphosphate synthase (farnesyl pyrophosphate synthetase, dimethylallyltranstransferase, geranyltranstransferase) (FDPS), mRNA), (XM_{—}034499, Homo sapiens farnesyl diphosphate synthase (farnesyl pyrophosphate synthetase, dimethylallyltranstransferase, geranyltranstransferase) (FDPS), mRNA), and (XM_{—}0345002, Homo sapiens farnesyl diphosphate synthase (farnesyl pyrophosphate synthetase, dimethylallyltranstransferase, geranyltranstransferase) (FOPS), mRNA).

Alternatively, FPP can also be made by adding IPP to GPP. Illustrative examples of nucleotide sequences encoding for an enzyme capable of this reaction include but are not limited to: (AE000657, Locus AAC06913; Aquifex aeolicus VF5), (NM_{—}202836, Arabidopsis thaliana), (D84432, Locus BAA12575; Bacillus subtilis), (112678, Locus AAC28894; Bradyrhizobium japonicum USDA 110), (BACFDPS; Geobacillus stearothermophilus), (NC0029407 Locus NP_{—}873754; Haemophilus ducreyi 35000HP), (L42023, Locus AAC23087; Haemophilus influenzae Rd KW20), (J05262; Homo sapiens), (YP_{—}395294; Lactobacillus sakei subsp. sakei 23K), (NC_{—}005823, Locus YP_{—}000273; Leptospira interrogans serovar Copenhageni str. Fiocruz L1-130), (AB003187; Micrococcus luteus), (NC_{—}002946, Locus YP_{—}208768; Neisseria gonorrhoeae FA 1090), (U00090, Locus AAB91752; Rhizobium sp. NGR234), (J05091; Saccharomyces cerevisae), (CP000031, Locus AAV93568; Silicibacter pomeroyi DSS-3), (AE008481, Locus AAK99890; Streptococcus pneumoniae R6), and (NC_{—}004556, Locus NP 779706; Xylella fastidiosa Temecula1).

FPP can then be subsequently converted to a variety of C₁₅ compounds. One illustrative example of a C₁₅ compound includes but is not limited to amorphadiene, whose structure is

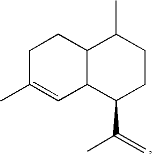

and is a precursor to artemisinin, which is made by Artemisia anna. Amorphadiene is typically made from FPP by amorphadiene synthase, a synthase that may be encoded by the heterologous sequences of the present invention. An illustrative example of a suitable nucleotide sequence is SEQ ID NO. 37 of U.S. Patent Publication No. 2004/0005678.

α-Farnesene, whose structure is

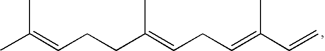

is typically made from FPP by α-farnesene synthase, and may be produced by the methods described herein. The synthase that may be encoded by heterologous sequences such as, but are not limited to DQ309034 from Pyrus communis cultivar d'Anjou (pear; gene name AFS1) and AY182241 from Malus domestica (apple; gene AFS1). Pechouus et al, Planta 219(1):84-94 (2004).

β-Farnesene, whose structure is

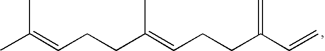

is typically made from FPP by β-farnesene synthase, and may be produced by the methods described herein. The synthase that may be encoded by heterologous sequences such as, but are not limited to: GenBank accession number AF024615 from Mentha×piperta (peppermint; gene Tspa11), and AY835398 from Artemisia annua. Picaud et al., Phytochemistry 66(9): 961-967 (2005) and may be used as heterologous sequences of the present invention.

Farnesol, whose structure is

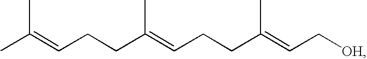

is typically made from FPP by a hydroxylase such as farnesol synthase. Farnesol may be produced through the use of heterologous sequences that may include but are not limited to GenBank accession number AF529266 from Zea mays and YDR481c from Saccharomyces cerevisiae (gene Pho8). Song, L., Applied Biochemistry and Biotechnology 128:149-158 (2006).

Nerolidol, whose structure is

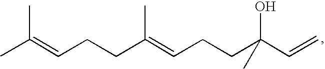

is also known as peruviol, and is typically made from FPP by a hydroxylase such as nerolidol synthase, that maybe encoded by heterologous sequences of the present invention. An illustrative example of a suitable nucleotide sequence that may be used as a heterologous sequence includes but is not limited to AF529266 from Zea mays (maize; gene tps1).

Patchoulol, whose structure is

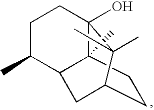

is typically made from FPP by patchouliol synthase. Patchoulol may be produced in the present invention by using heterologous sequences such as, but is not limited to AY508730 REGION: 1 . . . 1659 from Pogostemon cablin.

Valencene, whose structure is

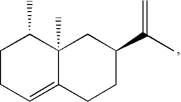

is typically made from FPP by nootkatone synthase. Lllustrative examples of a suitable nucleotide sequence that may be used to encode the synthase includes but is not limited to AF441124 REGION: 1 . . . 1647 from Citrus sinensis and AY917195 REGION: 1 . . . 1653 from Perilla frutescens.

Heterologous products can also include C₂₀ compounds, such as those derived from geranylgeraniol pyrophosphate (GGPP) which is made by the condensation of three molecules of IPP with one molecule of DMAPP. These C₂₀ compounds are also known as diterpenes because they are derived from four isoprene units. In certain embodiments, the host cells of the present invention comprises a heterologous sequence that encodes an enzyme that converts IPP and DMAPP into GGPP. An enzyme known to catalyze this step is, for example, geranylgeranyl pyrophosphate synthase.

Illustrative examples of nucleotide sequences for geranylgeranyl pyrophosphate synthase include but are not limited to: (ATHGERPYRS; Arabidopsis thaliana), (BT005328; Arabidopsis thaliana), (NM_{—}119845, Arabidopsis thaliana), (NZ_AAJM01000380, Locus ZP_{—}00743052; Bacillus thuringiensis serovar israelensis, ATCC 35646 sq1563), (CRGGPPS; Catharanthus roseus), (NZLAABF02000074, Locus ZP_{—}00144509; Fusobacterium nucleatum subsp. vincentii, ATCC 49256), (GFGGPPSGN; Gibberella fujikuroi), (AY371321; Ginkgo biloba), (ABO55496; Hevea brasiliensis), (AB017971; Homo sapiens), (MCI276129; Mucor circinelloides f. lusitanicus), (AB016044; Mus musculus), (AABX01000298, Locus NCU01427; Neurospora crassa), (NCU20940; Neurospora crassa), (NZ_AAKL01000008, Locus ZP_{—}00943566; Ralstonia solanacearum UW551), (AB118238; Rattus norvegicus), (SCU31632; Saccharomyces cerevisiae), (AB3016095; Synechococcus elongates), (SAGGPS; Sinapis alba), (SSOGDS; Sulfolobus acidocaldarius), (NC_{—}007759, Locus YP_{—}461832; Syntrophus aciditrophicus SB), and (NQC006840, Locus YP_{—}204095; Vibrio fischeri ES114).

Alternatively, GGPP can also be made by adding IPP to FPP. Illustrative examples of nucleotide sequences encoding an enzyme capable of this reaction include but are not limited to: (NM_{—}12315; Arabidopsis thaliana), (ERWCRTE; Pantoea agglomerans), (D90087, Locus BAA14124; Pantoea ananatis), (X52291, Locus CAA36538; Rhodobacter capsulatus), (AF195122, Locus AAF24294; Rhodobacter sphaeroides), and (NC_{—}004350, Locus NP-721015; Streptococcus mutans UA159). GGPP can then subsequently be converted to a variety of C₂₀ isoprenoids. Illustrative examples of C₂₀ compounds include for example, geranylgeraniol. Geranylgeraniol, whose structure is

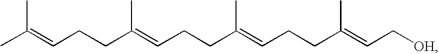

can be made by e.g., adding to the expression constructs a phosphatase gene after the gene for a GGPP synthase.

Abietadiene is another diterpene that may be produced by the methods described herein. Abietadiene encompasses the following isomers:

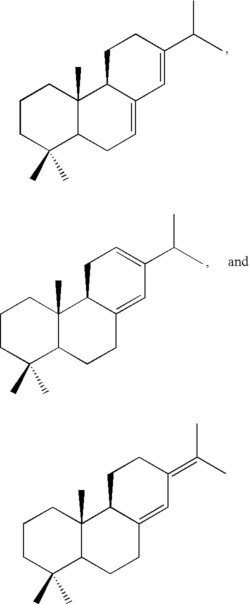

and is typically made by abietadiene synthase. Abietadience synthase may be encoded by a suitable heterologous nucleotide sequence including, but not limited to: (U50768; Abies grandis) and (AY473621; Picea abies).

C₂₀₊ compounds are also within the scope of the present invention. Illustrative examples of such compounds include sesterterpenes (C₂₅ compound made from five isoprene units), tritenes (C₃₀ compounds made from six isoprene units), and tetraterpenes (C₄₀ compound made from eight isoprene units). These compounds are made by using similar methods described herein and substituting or adding nucleotide sequences for the appropriate synthase(s). In some embodiments, the amount of heterologously produced product is greater than 10 mg/L. For example, in some embodiments, the amount of product produced by a cell of the invention is from about 10 mg/L to about 100 mg/L, from about 100 mg/L to about 1,000 mg/L, from about 1,000 mg/L to about 1,500 mg/L, from about 1,500 mg/L to about 2,000 mg/L, from about 2,000 mg/L to about 3,000 mg/L, from about 3,000 mg/L to about 4,000 mg/L, from about 4,000 mg/L to about 5,000 mg/L, from about 5,000 mg/L to about 6,000 mg/L, from about 6,000 mg/L to about 7,000 mg/L, from about 7,000 mg/L to about 8,000 mg/L, or from about 8,000 mg/L to about 10,000 mg/L. In certain embodiments, the amount of heterologously produced product is greater than 10,000 mg/L. In certain such embodiments, the amount of heterologously produced product is from about 10,000 mg/L to about 20,000 mg/L, from about 20,000 mg/L to about 30,000 mg/L, from about 30,000 mg/L to about 40,000 mg/L, or from about 40,000 mg/L to about 50,000 mg/L. In certain embodiments, the amount of heterologously produced product is greater than 50,000 mg/L. Production levels are expressed on a per unit volume (e.g., per liter) cell culture basis. The level of protein or compound produced is readily determined using well-known methods, e.g., gas chromatography-mass spectrometry, liquid chromatography-mass spectrometry, ion chromatography-mass spectrometry, thin layer chromatography, pulsed amperometric detection, and UV-vis spectrometry.

The heterologously produced protein, or compound made by such protein, can be recovered from the host cell or from the culture medium in which the host cell is grown using standard purification methods well known in the art, including, e.g., high performance liquid chromatography, gas chromatography, and other standard chromatographic methods. In some embodiments, the purified protein or compound is pure, e.g., at least about 40% pure, at least about 50% pure, at least about 60% pure, at least about 70% pure, at least about 80% pure, at least about 90% pure, at least about 95% pure, at least about 98%, or more than 98% pure, where the term “pure” refers to protein or compound that is free from side products, macromolecules, contaminants, etc

The heterologous products of the present invention may be commercially and industrially useful. For example, produced isoprenoids may be used as pharmaceuticals, cosmetics, perfumes, pigments and colorants, antibiotics, fungicides, antiseptics, nutraceuticals (e.g. vitamins), fine chemical intermediates, polymers, pheromones, industrial chemicals, and fuels.

In one embodiment, the isoprenoid produced is a vitamin such as Vitamin A, A, or K and other isoprenoid based nutrients. Vitamin K, an important vitamin involved in the blood coagulation system, which is utilized as a hemostatic agent. Vitamin K is also involved in osteo-metabolism, can be applied to the treatment of osteoporosis. In addition, ubiquinone and vitamin K are effective in inhibiting barnacles from clinging to objects, and so make a suitable additive to paint products to prevent barnacles from clinging.

The present invention also provides methods for the production of isoprenoids such as ubiquinone, which plays a role in vivo as an essential component of the electron transport system. Ubiquinone is useful not only as a pharmaceutical effective against cardiac diseases, but also as a beneficial food additive. Phylloquinone and menaquinone have been approved as pharmaceuticals.

The present invention also involves the production of carotenoids, such as β-carotene, astaxanthin, and cryptoxanthin, which are expected to possess cancer preventing and immunopotentiating activity. Carotenoids produced by these methods may also be used as pigments. Carotenoids represent one of the most widely distributed and structurally diverse classes of natural pigments, producing pigment colors of light yellow to orange to deep red. Examples of carotenogenic tissues include carrots, tomatoes, red peppers, and the petals of daffodils and marigolds. Carotenoids are synthesized by all photosynthetic organisms, as well as some bacteria and fungi. These pigments have important functions in photosynthesis, nutrition, and protection against photooxidative damage. For example, animals do not have the ability to synthesize carotenoids but must instead obtain these nutritionally important compounds through their dietary sources. One specific isoprenoid, such as β-carotene (yellow-orange) or astaxanthin (red-orange), can serve to enhance flower color or nutriceutical composition. For example, modified cyanidin and delphinidin anthocyanin pigments may be produced and used to produce shades in red to blue groupings. Lutein and zeaxanthin can be produced, and used in combination with colorless flavonols (Nielsen and Bloor, Scienia Hort. 71:257-266, 1997).

The present invention also encompasses the heterologous production of lipids other than terpenoids. For examples, lipids such as fatty acyls (including fatty acids), glycerolipids, glycerophospholipids, sphingolipids, sterol lipids, prenol lipids, saccharolipids and polyktides. Production of carbohydrates, such as monosaccarides, disaccharides, and polysaccharides.

### Host Cells

Any host cell may be used in the practice of the present invention. The host cell comprises a galactose induction machinery. Illustrative examples of suitable host cells include prokaryotic and eukaryotic cells, such as archae cells, bacterial cells, and fungal cells. In many embodiments, the host cell can be grown in liquid growth medium.

Some non-limiting examples of archae cells include those belonging to the genera: Aeropyrum, Archaeglobus, Hatobacterium, Methanococcus, Methanobacterium, Pyrococcus, Sulfolobus, and Thermoplasma. Some non-limiting examples of archae strains include Aeropyrum pernix, Archaeoglobus fulgidus, Methanococcus jannaschii, Methanobacterium thermoautotrophicum, Pyrococcus abyssi, Pyrococcus horikoshii, Thermoplasma acidophilum, and Thernoplasma volcanium.

Some non-limiting examples of bacterial cells include those belonging to the genera: Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas.

Some non-limiting examples of bacterial strains include Bacillus subtilis, Bacillus amyloliquefacines, Brevibacterium ammoniagenes, Brevibacterium immariophilum, Clostridium beigerinckii, Enterobacter sakazakii, Escherichia coli, Lactococcus lactis, Mesorhizobium loti, Pseudomonas aeruginosa, Pseudomonas mevalonii, Pseudomonas pudica, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

If a bacterial host cell is used, a non-pathogenic strain, such as non-limiting examples Bacillus subtilis, Escherichia coli Lactibacillus acidophilus, Lactobacillus helveticus, Pseudomonas aeruginosa, Pseudomonas mevalonii, Pseudomonas pudita, Rhodobacter sphaeroides, Rodobacter capsulatus, and Rhodospirillum rubrum may be used.

Some non-limiting examples of eukaryotic cells include fungal cells. Some non-limiting examples of fungal cells include those belonging to the genera: Aspergillus, Candida, Chrysosporium, Cryotococcus, Fusarium, Kluyveromyces, Neotyphodium, Neurospora, Penicillium, Pichia, Saccharomyces, and Trichoderma.

Some non-limiting examples of eukaryotic strains include Aspergillus nidulans, Aspergillus niger, Aspergillus oryzae, Candida albicans, Chrysosporium lucknowense, Fusarium graminearum, Fusarium venenatum, Fusarium sp., Hansenula polymorpha, Kluyveromyces sp., Kluyveromyces lactis, Neurospora crassa, Pichia angusta, Pichia finlandica, Pichia kodamae, Pichia membranaefaciens, Pichia methanolica, Pichia opuntiae, Pichia pastoris, Pichiapijperi, Pichia quercuum, Pichia salictaria, Pichia thermotolerans, Pichia trehalophila, Pichia stipitis, Pichia sp., Streptomyces ambofaciens, Streptomyces aureofaciens, Streptomyces aureus, Saccaromyces bayanus, Saccaromyces boulardi, Saccharomyces cerevisiae, StreptomycesfuJngicidicus, Streptomyces griseochromogenes, Streptomyces griseus, Streptomyces lividans, Streptomyces olivogriseus, Streptomyces rameus, Streptomyces tanashiensis, Streptomyces vinaceus, Saccharomyces sp., and Trichoderma reesei.

If a eukaryotic host cell is used, a non-pathogenic strain, such as non-limiting examples Fusarium graminearum, Fusarium venenatum, Pichia pastoris, Saccaromyces boulardi, and Saccaromyces cerevisiae, may be used.

In addition, certain strains have been designated by the Food and Drug Administration as GRAS or Generally Regarded As Safe and maybe used in the present invention. Some non-limiting examples of these strains include Bacillus subtilis, Lactibacillus acidophilus, Lactobacillus helveticus, and Saccharomyces cerevisiae.

In certain embodiments, the host cell may have a defective galactose catabolism pathway. For example, one or more endogenous enzymes that mediate galactose catabolism is functionally disabled. Without being bound by theory, disabling galactose catabolism can permit more galactose to be available for induction of the galactose-inducible promoter. The functional disablement can be achieved in any of a variety of ways known in the art, including by deleting all or a part of a gene such that the gene product is not made or is truncated and is enzymatically inactive; mutating a gene such that the gene product is not made or is truncated and is enzymatically non-functional; inserting a mobile genetic element into a gene such that the gene product is not made or is truncated and is enzymatically non-functional; and deleting or mutating one or more regulatory elements that control expression of a gene such that the gene product is not made. Suitable enzymes that when functionally disabled eliminate or reduce the ability of a Saccharomyces cerevisiae cell to catabolize galactose include GAL1p (GenBank Locus YBR020W), GAL7p (GenlBank Locus YBR018C), and GAL10p (GenBank Locus YBR019C), and other functional homologs.

### Nucleic Acids

In many embodiments, the host cell is a genetically modified cell in which heterologous nucleic acid molecules have been inserted, deleted, or modified (i.e., mutated; e.g., by insertion, deletion, substitution, and/or inversion of nucleotides).

In certain embodiments, the heterologous nucleic acids are inserted into an expression vectors. The choice of expression vector will depend on the choice of host cells. A number of expression vectors suitable for expression in eukaryotic cells including yeast, avian, and mammalian cells are known in the art, many of which are commercially available. Some examples of common vectors include but are not limited to YEpl3 and the Sikorski series pRS303-306, 313-316, 423-426.

In certain embodiments, a nucleotide sequence comprising a galactose-inducible expression cassette and a nucleotide sequence encoding a galactose transporter are present on a single expression vector. In other embodiments, a nucleotide sequence comprising a galactose-inducible expression cassette and a nucleotide sequence encoding a galactose transporter are present on two expression vectors. In certain embodiments, a nucleotide sequence comprising a galactose-inducible expression cassette and a nucleotide sequence encoding a lactose transporter are present on a single expression vector. In other embodiments, a nucleotide sequence comprising a galactose-inducible expression cassette and a nucleotide sequence encoding a lactose transporter are present on two expression vectors. In certain embodiments, a nucleotide sequence comprising a galactose-inducible expression cassette and a nucleotide sequence encoding a lactase are present on a single expression vector. In other embodiments, a nucleotide sequence comprising a galactose-inducible expression cassette and a nucleotide sequence encoding a lactase are present on two expression vectors.

In certain embodiments, a nucleotide sequence comprising a galactose-inducible expression cassette, a nucleotide sequence encoding a galactose transporter, and a nucleotide sequence encoding a lactase are present on a single expression vector. In other embodiments, a nucleotide sequence comprising a galactose-inducible expression cassette, a nucleotide sequence encoding a galactose transporter, and a nucleotide sequence encoding a lactase are present on two or more expression vectors. In certain embodiments, a nucleotide sequence comprising a galactose-inducible expression cassette, a nucleotide sequence encoding a lactase, and a nucleotide sequence encoding a lactose transporter are present on a single expression vector. In other embodiments, a nucleotide sequence comprising a galactose-inducible expression cassette, a nucleotide sequence encoding a lactase, and a nucleotide sequence encoding a lactose transporter are present on two or more expression vectors.

In certain embodiments, the host cell comprises a single heterologous galactose-inducible expression cassette. In other embodiments, the host cell comprises a plurality of heterologous galactose-inducible expression cassettes. In certain embodiments, the cell comprises a single nucleotide sequence encoding a galactose transporter. In other embodiments, the host cell comprises a plurality of nucleotide sequences encoding one or more galactose transporters. In certain embodiments, the host cell comprises a single nucleotide sequence encoding a lactose transporter. In other embodiments, the host cell comprises a plurality of nucleotide sequences encoding one or more lactose transporters. In certain embodiments, the host cell comprises a single nucleotide sequence encoding a lactase. In other embodiments, the host cell comprises a plurality of nucleotide sequence encoding one or more lactases. The plurality of nucleotide sequences encoding one or more proteins may be on a single or multiple expression vectors. The proteins may be the same or different, and may further be provided on the same or different expression vector as one or more heterologous galactose-inducible expression cassette.

In some embodiments, the expression vectors are extra-chromosomal expression vectors. In some embodiments the expression vectors are episomal. For example, the host cell may comprise one or more heterologous galactose-inducible expression cassettes on an extra-chromosomal expression vector or on an episomal vector. In certain embodiments, the host cell comprises one or more copies of nucleotide sequences encoding a galactose transporter on an extra-chromosomal expression vector or an episomal vector. In some embodiments, the host cell comprises one or more copies of nucleotide sequences encoding a lactose transporter on an extra-chromosomal expression vector. In some embodiments, the host cell comprises one or more copies of nucleotide sequences encoding a lactase on an extra-chromosomal expression vector or episomal vector. In some embodiments, the extra-chromosomal expression vector may have a plurality of proteins encoded by a single expression vector. For example, a single extra-chromosomal expression vector or episomal vector may comprise a nucleotide sequence encoding a lactose transporter and a nucleotide sequence encoding lactase. In some embodiments, a single extra-chromosomal expression vector may comprise mutliple copies of nucleotide sequences encoding the same protein, for example a single extra-chromosomal expression vector may have two nucleotide sequences encoding a single lactase. In other embodiments, the single extra-chromosomal expression vector may comprise one or more galactose inducible expression cassettes with one or more other nucleotide sequences that encode a lactase, lactose transporter, or galactose transporter.

In other embodiments, the expression vectors are chromosomal integration vectors, wherein the heterologous nucleotide sequences of the chromosomal integration vectors are introduced into the chromosomes of the host cells, or into the genome of the host cell. In some embodiments, the host cell comprises the one or more heterologous galactose-inducible expression cassettes integrated into a chromosome. In some embodiments, the host cell comprises the one or more copies of nucleotide sequences encoding a galactose transporter integrated into a chromosome. In some embodiments, the host cell comprises the one or more copies of nucleotide sequences encoding a lactose transporter integrated into a chromosome. In some embodiments, the host cell comprises the one or more copies of nucleotide sequences encoding a lactase integrated into a chromosome. In some embodiments, the chromosomal intergration vector comprises sequences for one or more heterologous galactose-inducible expression vector and one or more other nucleotides sequences encoding one or more lactases, lactose transporters, or galactose transporters, that are integrated into a chromosome.

In certain embodiments, a nucleotide sequence encoding a galactose or lactose transporter and a nucleotide sequence encoding a lactase are operably linked to the same regulatory elements. In other embodiments, a nucleotide sequence encoding a galactose or lactose transporter is under control of a first regulatory element, and a nucleotide sequence encoding a lactase is under control of a second regulatory element. Regulatory elements may be promoters. For example, the promoters may be inducible or constitutive. Suitable inducible promoters include but are not limited to the promoters of the Saccharomyces cerevisiae genes ADH2, PHr5, CUPr, MET25, M-ET3, CYC1, HIS3, GAPDH, ADC1, TRP1, URA3, LEU2, TP1, and AOX1. In other embodiments, the promoter is constitutive. Suitable constitutive promoters include but are not limited to Saccharomyces cerevisiae genes PGK1, TDH1, TDHS3, FBA 1, ADH1, LEU2, ENO, TPI1, and PYK1. To generate a genetically modified host cell, one or more heterologous nucleic acids are introduced stably or transiently into a cell, using established techniques, including but not limited to electroporation, calcium phosphate precipitation, DEAE-dextran mediated transfection, and liposome-mediated transfection. For stable transformation, a nucleic acid will generally further include a selectable marker (e.g., a neomycin resistance, ampicillin resistance, tetracycline resistance, chloramphenicol resistance, or kanamycin resistance marker). Stable transformation can also be selected for using a nutritional marker gene that confers prototrophy for an essential amino acid (e.g., the Saccharomyces cerevisiae nutritional marker genes URA3, HIS3, LEU2, MET2, and LYS2, other may include the HISM or KANMX.

### Variant Enzymes and Nucleotide Sequence Homologs

The coding sequence of any known protein of the invention may be altered in various ways known in the art to generate variant proteins comprising targeted changes in the amino acid sequence but not substantially altering the function of the protein. The sequence changes may be substitutions, insertions, or deletions. Also suitable for use are nucleic acid homologs comprising nucleotide sequences having at least about 70%, at least about 75%, at least about 80%, at least about 85%, at least about 90%, at least about 95%, at least about 98%, or at least about 99% nucleotide sequence identity to nucleotide sequences of the invention.

It is understood that equivalents or variants of the wild-type polypeptide or protein also are within the scope of this invention. The terms “equivalent”, “functional homolog”, and “biologically active fragment thereof” are used interchangeably and refer to variants from a selected sequence by any combination of additions, deletions, or substitutions while preserving at least one functional property of the fragment relevant to the context in which it is being used. For instance, an equivalent of a proteinaceous enzyme (e.g., lactase) may have the same or comparable ability to catalyze a given chemical reaction as compared to a wild-type proteinaceous enzyme. As is apparent to one skilled in the art, the equivalent may also be associated with, or conjugated with, other substances or agents to facilitate, enhance, or modulate its function. The invention includes modified polypeptides containing conservative or non-conservative substitutions that do not significantly affect their properties, such as enzymatic activity of the peptides or their tertiary structures. Modification of polypeptides is routine practice in the art. Amino acid residues which can be conservatively substituted for one another include but are not limited to: glycine/alanine; valine/isoleucine/leucine; asparagine/glutamine; aspartic acid/glutamic acid; serine/threonine; lysine/arginine; and phenylalanine/tryosine. These polypeptides also include glycosylated and nonglycosylated polypeptides, as well as polypeptides with other post-translational modifications, such as, for example, glycosylation with different sugars, acetylation, and phosphorylation.

### Codon Usage

In some embodiments, a nucleotide sequence used to generate a host cell of the invention is modified such that the nucleotide sequence reflects the codon preference for the cell. In certain embodiments, the nucleotide sequence will be modified for yeast codon preference (see, e.g., Bennetzen and Hall. 1982. J. Biol. Chem. 257(6): 3026-3031).

### Kits

The present invention also encompasses kits that provide reagents for producing heterologous products through galactose-inducible production of heterologous sequences without direct supplementation of galactose to the cell culture medium. The kit provides reagents such that the amount of product obtained is comparable to that obtained by culturing the host cell in a medium supplemented with comparable moles of galactose. For example, the amount of product produced by lactose-supplemented medium is comparable to that produced from a medium supplemented with comparable quantity of galactose. In some embodiments, the amount of product produced is approximately equal to or greater than the amount of product obtained from a medium directly supplemented with comparable moles of galactose. In some embodiments, the amount of product produced is at least 1.2 fold, 1.5 fold, 2 fold (ie. double), 2.5 fold, 3 fold, 4, fold, 5 fold or more than the amount of product obtained from a medium supplemented with comparable moles of galactose.

Each kit typically comprises reagents that render the production of heterologous products through a galactose-inducible regulatory cassette without directly supplementing galactose to the cell culture medium. In one embodiment, the kit may comprise components for a galactose-inducible expression system. For example, the kit may comprise galactose-inducible regulatory elements that may be operably linked to a heterologous sequence of choice. The kit may further comprise reagents such as cloning reagents for linking the heterologous sequence of choice to the regulatory element. In other embodiments, the kit may further comprise galactose-inducible expression vectors, wherein a heterologous sequence of choice can be inserted. The vectors can be episomal, extrachromosomal or for chromosomal integration. In other embodiments, the kits can comprise vectors for expression lactase, lactase transporters, and/or galactose transporters. In other embodiments, the kid may comprise components for expressing the galactose induction machinery. Different kits may be formulated for different host cell types. For example, some kits may comprise reagents for host cells with endogenous lactase, and thus, the kit may not comprise a vector expressing lactase.

In some embodiments, the kits comprise a set of expression vectors comprising at least a first expression vector and at least a second expression vector, wherein the first expression vector comprises a first heterologous sequence operably linked to a galactose-inducible regulatory element, and a second expression vector comprise a second heterologous sequence encoding a lactase or biologically active fragment thereof.

In other embodiments, the kits may further comprise host cells. In other embodiments, the kits further comprise culture medium, compounds for inducing production of heterologous products, and other cell culture supplies.

Each reagent in a kit can be supplied in a solid form or dissolved/suspended in a liquid buffer suitable for inventory storage, and later for exchange or addition into the reaction medium when the test is performed. Suitable individual packaging is normally provided. The kit can optionally provide additional components that are useful in the procedure. These optional components include, but are not limited to, buffers, purifying reagents, harvesting reagents, means for detection, control samples, control compounds (such as galactose), instructions, and interpretive information.

The kits of the present invention typically comprise instructions for use of reagents contained therein. The instructions can be provided in form of product inserts, manual, recorded in any readable medium including electronic medium.

### EXAMPLES

The practice of the present invention can employ, unless otherwise indicated, conventional techniques of the biosynthetic industry and the like, which are within the skill of the art. To the extent such techniques are not described fully herein, one can find ample reference to them in the scientific literature.

In the following examples, efforts have been made to ensure accuracy with respect to numbers used (for example, amounts, temperature, and so on), but variation and deviation can be accommodated, and in the event a clerical error in the numbers reported herein exists, one of ordinary skill in the arts to which this invention pertains can deduce the correct amount in view of the remaining disclosure herein. Unless indicated otherwise, temperature is reported in degrees Celsius, and pressure is at or near atmospheric pressure at sea level. All reagents, unless otherwise indicated, were obtained commercially. The following examples are intended for illustrative purposes only and do not limit in any way the scope of the present invention.

### Example 1

This example describes methods for making plasmids for the targeted integration of heterologous nucleic acids comprising galactose-inducible promoters operably linked to protein coding sequences into specific chromosomal locations of Saccharomyces cerevisiae.

Genomic DNA was isolated from Saccharomyces cerevisiae strains Y002 (CEN.PK2 background MATA ura3-52 trp1-289 leu2-3, 112 his3Δ1 MAL2-8C SUC2), Y007 (S288C background MATA trp1Δ63), Y051 (S288C background MATα his3Δ1 leu2Δ0 lys2Δ0 ura3Δ0 P_{GAL1}-HMG1¹⁵⁸⁶⁻³²³³ P_{GAL1}-upc2-1 erg9::P_{MET3}-ERG9::HIS3 P_{GAL1}-ERG20 P_{GAL1}-HMG1¹⁵⁸⁶⁻³³²³) and EG123 (MATA ura3 trp1 leu2 his4 can1). The strains were grown overnight in liquid medium containing 1% Yeast extract, 2% Bacto-peptone, and 2% Dextrose (YPD medium). Cells were isolated from 10 mL liquid cultures by centrifugation at 3,100 rptm, washing of cell pellets in 10 mL ultra-pure water, and re-centrifugation. Genomic DNA was extracted using the Y-DER yeast DNA extraction kit (Pierce Biotechnologies, Rockford, Ill.) as per manufacturer's suggested protocol. Extracted genomic DNA was re-suspended in 100 uL 10 mM Tris-Cl, pH 8.5, and OD_{260/280} so readings were taken on a ND-1000 spectrophotometer (NanoDrop Technologies, Wilmington, Del.) to determine genomic DNA concentration and purity.

DNA amplification by Polymerase Chain Reaction (PCR) was done in an Applied Biosystems 2720 Thermocycler (Applied Biosystems Inc, Foster City, Calif.) using the Phusion High Fidelity DNA Polymerase system (Finnzymes OY, Espoo, Finland) as per manufacturer's suggested protocol. Upon the completion of a PCR amplification of a DNA fragment that was to be inserted into the TOPO TA pCR2.1 cloning vector (Invitrogen, Carlsbad, Calif.). A nucleotide overhangs were created by adding 1 uL of Qiagen Taq Polymerase (Qiagen, Valencia, Calif.) to the reaction mixture and performing an additional 10 minute, 72° C. PCR extension step, followed by cooling to 4° C. Upon completion of PCR amplification, 8 uL of a 50% glycerol solution was added to the reaction mix, and the entire mixture was loaded onto a 1% TBE (0.89 M Tris, 0.89 M Boric acid, 0.02 M EDTA sodium salt) agarose gel containing 0.5 ug/nL ethidium bromide.

Agarose gel electrophoresis was performed at 120 V, 400 mA for 30 minutes, and DNA bands were visualized using ultraviolet light. DNA bands were excised from the gel with a sterile razor blade, and the excised DNA was gel purified using the Zymoclean Gel DNA Recovery Kit (Zymo Research, Orange, Calif.) according to manufacturer's suggested protocols. The purified DNA was eluted into 10 uL ultra-pure water, and OD_{260/280} readings were taken on a ND-1000 spectrophotometer to determine DNA concentration and purity.

Ligations were performed using 100-500 ug of purified PCR product and High Concentration T4 DNA Ligase (New England Biolabs, Ipswich, Mass.) as per manufacturer's suggested protocol. For plasmid propagation, ligated constructs were transformed into Escherichia coli DH5α chemically competent cells (Invitrogen, Carlsbad, Calif.) as per manufacturer's suggested protocol. Positive transformants were selected on solid media containing 1.5% Bacto Agar, 1% Tryptone, 0.5% Yeast Extract, 1% NaCl, and 50 ug/mL of an appropriate antibiotic. Isolated transformants were grown for 16 hours in liquid LB medium containing 50 ug/mL carbenicillin or kanamycin antibiotic at 37° C., and plasmid was isolated and purified using a QIAprep Spin Miniprep kit (Qiagen, Valencia, Calif.) as per manufacturer's suggested protocol. Constructs were verified by performing diagnostic restriction enzyme digestions, resolving DNA fragments on an agarose gel, and visualizing the bands using ultraviolet light. Select constructs were also verified by DNA sequencing, which was done by Elim Biopharmaceuticals Inc. (Hayward, Calif.).

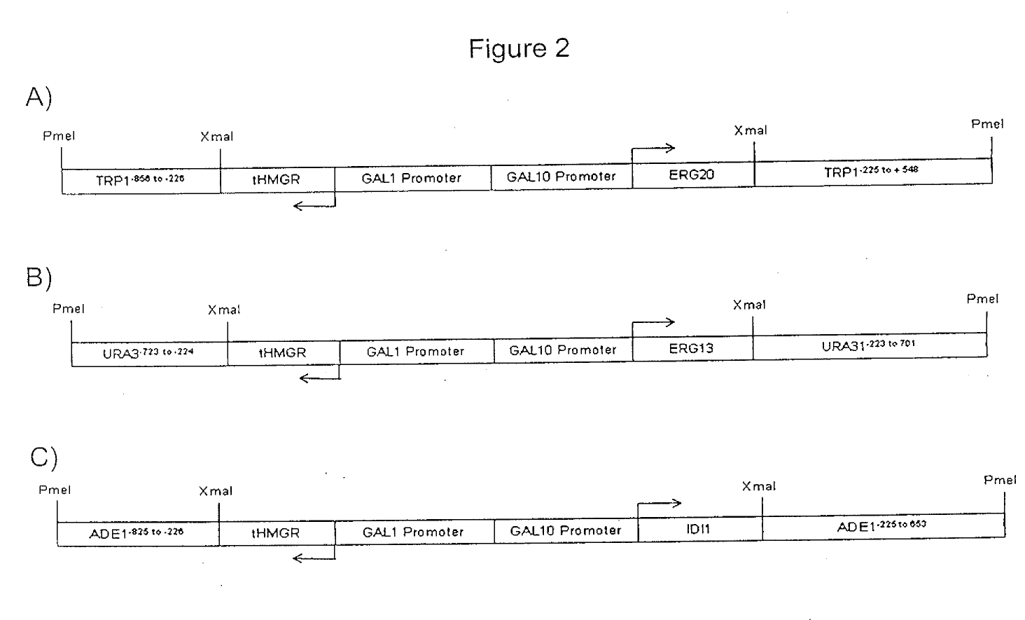

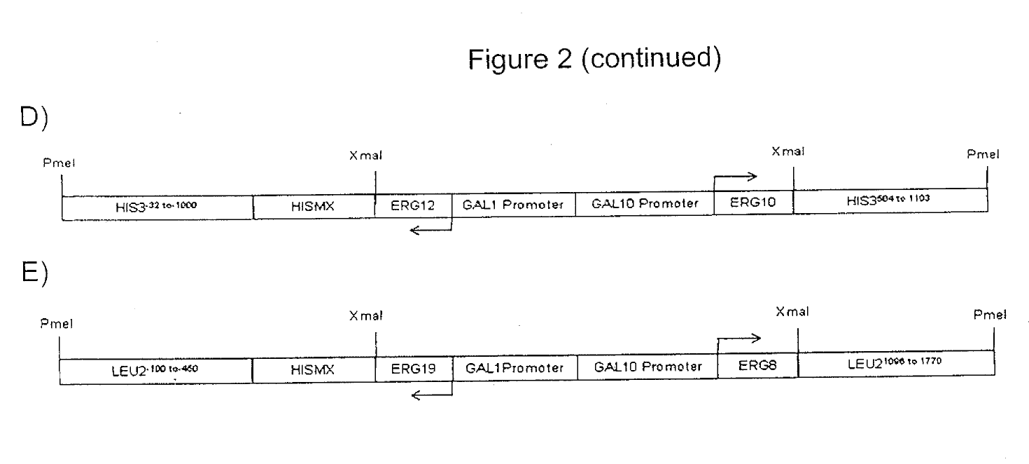

Plasmid pAM489 was generated by inserting the ERG20-P_{GAL}-tHMGR insert of vector pAM471 into vector pAM466. Vector pAM471 was generated by inserting DNA fragment ERG20-P_{GAL}-tHMGR, which comprises the open reading frame (ORF) of the ERG20 gene of Saccharomyces cerevisiae (ERG20 nucleotide positions 1 to 1208; A of ATG start codon is nucleotide 1) (ERG20), the genomic locus containing the divergent GAL1 and GAL10 promoter of Saccharomyces cerevisiae (GAL1 nucleotide position −1 to −668) P_{GAL}, and a truncated ORF of the HMG1 gene of Saccharomyces cerevisiae (HMG1 nucleotide positions 1586 to 3323) (tHMGR), into the TOPO Zero Blunt II cloning vector (Invitrogen, Carlsbad, Calif.). Vector pAM466 was generated by inserting DNA fragment TRP1^{-856 to +548}, which comprises a segment of the wild-type TRP1 locus of Saccharomyces cerevisiae that extends from nucleotide position −856 to position 548 and harbors a non-native internal XmaI restriction site between bases −226 and −225, into the TOPO TA pCR2.1 cloning vector (Invitrogen, Carlsbad, Calif.). DNA fragments ERG20-P_{GAL}-tHMGR and TRP1^{-856 to +548} were generated by PCR amplification as outlined in Table 1. FIG. 2A shows a map of the ERG20-P_{GAL}-tHMGR insert, and SEQ ID NO: 5 shows the nucleotide sequence of the DNA fragment. For the construction of pAM489, 400 ng of pAM471 and 100 ng of pAM466 were digested to completion using XmaI restriction enzyme (New England Biolabs, Ipswich, Mass.), DNA fragments corresponding to the ERG20-P_{GAL}-tHMGR insert and the linearized pAM466 vector were gel purified, and 4 molar equivalents of the purified insert was ligated with 1 molar equivalent of the purified linearized vector, yielding pAM489.

TABLE 1
PCR amplifications performed to generate pAM489

| PCR | | | | |
|---|---|---|---|---|
| Round | Template | Primer 1 | Primer 2 | PCR Product |
| 1 | 100 ng of Y051 genomic DNA | 61-67-CPK001-G | 61-67-CPK002-G | TRP1^{-856 to -226} |
| | | (SEQ ID NO: 30) | (SEQ ID NO: 31) | |
| | | 61-67-CPK003-G | 61-67-CPK004-G | TRP1^{-225 to +548} |
| | | (SEQ ID NO: 32) | (SEQ ID NO: 33) | |
| | 100 ng of EG123 genomic DNA | 61-67-CPK025-G | 61-67-CPK050-G | ERG20 |
| | | (SEQ ID NO: 54) | (SEQ ID NO: 62) | |
| | 100 ng of Y002 genomic DNA | 61-67-CPK051-G | 61-67-CPK052-G | P_{GAL} |
| | | (SEQ ID NO: 63) | (SEQ ID NO: 64) | |
| | | 61-67-CPK053-G | 61-67-CPK031-G | tHMGR |
| | | (SEQ ID NO: 65) | (SEQ ID NO: 55) | |
| 2 | 100 ng each of TRP1^{-856 to -226} and | 61-67-CPK001-G | 61-67-CPK004-G | TRP1^{-856 to +548} |
| | TRP1^{-225 to +548} purified PCR products | (SEQ ID NO: 30) | (SEQ ID NO: 33) | |
| | 100 ng each of ERG20 and P_{GAL} | 61-67-CPK025-G | 61-67-CPK052-G | ERG20-P_{GAL} |
| | purified PCR products | (SEQ ID NO: 54) | (SEQ ID NO: 64) | |
| 3 | 100 ng each of ERG20-P_{GAL} and | 61-67-CPK025-G | 61-67-CPK031-G | ERG20-P_{GAL}- |
| | tHMGR purified PCR products | (SEQ ID NO: 54) | (SEQ ID NO: 55) | tHMGR |

Plasmid pAM491 was generated by inserting the ERG13-P_{GAL}-tHMGR insert of vector pAM472 into vector pAM467. Vector pAM472 was generated by inserting DNA fragment ERG13-P_{GAL}-tHMGR, which comprises the ORF of the ERG13 gene of Saccharomyces cerevisiae (ERG13 nucleotide positions 1 to 1626) (ERG13), the genomic locus containing the divergent GAL1 and GAL10 promoter of Saccharomyces cerevisiae (GAL1 nucleotide position −1 to −668) (P_{GAL}), and a truncated ORF of the HMG1 gene of Saccharomyces cerevisiae (HMG1 nucleotide position 1586 to 3323) (tHMGR), into the TOPO Zero Blunt II cloning vector. Vector pAM467 was generated by inserting DNA fragment URA3^{-723 to 701}, which comprises a segment of the wild-type URA3 locus of Saccharomyces cerevisiae that extends from nucleotide position −723 to position −224 and harbors a non-native internal XmaI restriction site between bases -224 and -223, into the TOPO TA pCR2.1 cloning vector. DNA fragments ERG13-P_{GAL}-tHMGR and URA3^{-723 to 701} were generated by PCR amplification as outlined in Table 2. FIG. 2B shows a map of the ERG13-P_{GAL}-tHMGR insert, and SEQ ID NO: 6 shows the nucleotide sequence of the DNA fragment. For the construction of pAM491, 400 ng of pAM472 and 100 ng of pAM467 were digested to completion using XmaI restriction enzyme, DNA fragments corresponding to the ERG13-P_{GAL}-tHMGR insert and the linearized pAM467 vector were gel purified, and 4 molar equivalents of the purified insert was ligated with 1 molar equivalent of the purified linearized vector, yielding pAM491.

TABLE 2
PCR amplifications performed to generate pAM491

| PCR | | | | |
|---|---|---|---|---|
| Round | Template | Primer 1 | Primer 2 | PCR Product |
| 1 | 100 ng of Y007 genomic DNA | 61-67-CPK005-G | 61-67-CPK006-G | URA3^{-723 to -224} |
| | | (SEQ ID NO: 34) | (SEQ ID NO: 35) | |
| | | 61-67-CPK007-G | 61-67-CPK008-G | URA3^{-223 to 701} |
| | | (SEQ ID NO: 36) | (SEQ ID NO: 37) | |
| | 100 ng of Y002 genomic DNA | 61-67-CPK032-G | 61-67-CPK054-G | ERG13 |
| | | (SEQ ID NO: 56) | (SEQ ID NO: 66) | |
| | | 61-67-CPK052-G | 61-67-CPK055-G | P_{GAL} |
| | | (SEQ ID NO: 64) | (SEQ ID NO: 67) | |
| | | 61-67-CPK031-G | 61-67-CPK053-G | tHMGR |
| | | (SEQ ID NO: 55) | (SEQ ID NO: 65) | |
| 2 | 100 ng each of URA3^{-723 to -224} and | 61-67-CPK005-G | 61-67-CPK008-G | URA3^{-723 to 701} |
| | URA3^{-223 to 701} purified PCR products | (SEQ ID NO: 34) | (SEQ ID NO: 37) | |
| | 100 ng each of ERG13 and P_{GAL} | 61-67-CPK032-G | 61-67-CPK052-G | ERG13-P_{GAL} |
| | purified PCR products | (SEQ ID NO: 56) | (SEQ ID NO: 64) | |
| 3 | 100 ng each of ERG13-P_{GAL} and | 61-67-CPK031-G | 61-67-CPK032-G | ERG13-P_{GAL}- |
| | tHMGR purified PCR products | (SEQ ID NO: 55) | (SEQ ID NO: 56) | tHMGR |

Plasmid pAM493 was generated by inserting the IDI1-P_{GAL}-tHMGR insert of vector pAM473 into vector pAM468. Vector pAM473 was generated by inserting DNA fragment IDI1-P_{GAL}-tHMGR, which comprises the ORF of the IDI1 gene of Saccharomyces cerevisiae (IDI1 nucleotide position 1 to 1017) (IDI1), the genomic locus containing the divergent GAL1 and GAL10 promoter of Saccharomyces cerevisiae (GAL1 nucleotide position −1 to −668) (P_{GAL}), and a truncated ORF of the HMG1 gene of Saccharomyces cerevisiae (HMG1 nucleotide positions 1586 to 3323) (tHMGR), into the TOPO Zero Blunt II cloning vector. Vector pAM468 was generated by inserting DNA fragment ADE1^{-825 to 653}, which comprises a segment of the wild-type ADE1 locus of Saccharomyces cerevisiae that extends from nucleotide position −225 to position 653 and harbors a non-native internal XmaI restriction site between bases −226 and −225, into the TOPO TA pCR2.1 cloning vector. DNA fragments IDI1-P_{GAL}-tHMGR and ADE1^{-825 to 653} were generated by PCR amplification as outlined in Table 3. FIG. 2C shows a map of the IDI1-P_{GAL}-tHMGR insert, and SEQ ID NO: 7 shows the nucleotide sequence of the DNA fragment. For the construction of pAM493, 400 ng of pAM473 and 100 ng of pAM468 were digested to completion using XmaI restriction enzyme, DNA fragments corresponding to the IDI1-P_{GAL}-tHMGR insert and the linearized pAM468 vector were gel purified, and 4 molar equivalents of the purified insert was ligated with 1 molar equivalent of the purified linearized vector, yielding vector pAM493.

TABLE 3
PCR amplifications performed to generate pAM493

| PCR | | | | |
|---|---|---|---|---|
| Round | Template | Primer 1 | Primer 2 | PCR Product |
| 1 | 100 ng of Y007 genomic DNA | 61-67-CPK009-G | 61-67-CPK010-G | ADE1^{-825 to -226} |
| | | (SEQ ID NO: 38) | (SEQ ID NO: 39) | |
| | | 61-67-CPK011-G | 61-67-CPK012-G | ADE1^{-225 to 653} |
| | | (SEQ ID NO: 40) | (SEQ ID NO: 41) | |
| | 100 ng of Y002 genomic DNA | 61-67-CPK047-G | 61-67-CPK064-G | IDI1 |
| | | (SEQ ID NO: 61) | (SEQ ID NO: 76) | |
| | | 61-67-CPK052-G | 61-67-CPK065-G | P_{GAL} |
| | | (SEQ ID NO: 64) | (SEQ ID NO: 77) | |
| | | 61-67-CPK031-G | 61-67-CPK053-G | tHMGR |
| | | (SEQ ID NO: 55) | (SEQ ID NO: 65) | |
| 2 | 100 ng each of ADE1^{-825 to -226} and | 61-67-CPK009-G | 61-67-CPK012-G | ADE1^{-825 to 653} |
| | ADE1^{-225 to 653} purified PCR products | (SEQ ID NO: 38) | (SEQ ID NO: 41) | |
| | 100 ng each of IDI1 and P_{GAL} purified | 61-67-CPK047-G | 61-67-CPK052-G | IDI1-P_{GAL} |
| | PCR products | (SEQ ID NO: 61) | (SEQ ID NO: 64) | |
| 3 | 100 ng each of IDI1-P_{GAL} and tHMGR | 61-67-CPK031-G | 61-67-CPK047-G | IDI1-P_{GAL}-tHMGR |
| | purified PCR products | (SEQ ID NO: 55) | (SEQ ID NO: 61) | |

Plasmid pAM495 was generated by inserting the ERG10-P_{GAL}-ERG12 insert of pAM474 into vector pAM469. Vector pAM474 was generated by inserting DNA fragment ERG10-P_{GAL}-ERG12, which comprises the ORF of the ERG10 gene of Saccharomyces cerevisiae (ERG10 nucleotide position 1 to 1347) (ERG10), the genomic locus containing the divergent GAL1 and GAL10 promoter of Saccharomyces cerevisiae (GAL1 nucleotide position −1 to −668) P_{GAL}), and the ORF of the ERG12 gene of Saccharomyces cerevisiae (ERG12 nucleotide position 1 to 1482) (ERG12), into the TOPO Zero Blunt II cloning vector. Vector pAM469 was generated by inserting DNA fragment HIS3^{-32 to -1000}-HISMX-HIS3^{504 to -}1103 which comprises two segments of the HIS locus of Saccharomyces cerevisiae that extend from nucleotide position −32 to position −1000 and from nucleotide position 504 to position 1103, a HISMX marker, and a non-native XmaI restriction site between the HIS3^{504 to -1103} sequence and the HISMX marker, into the TOPO TA pCR2.1 cloning vector. DNA fragments ERG10-P_{GAL}-ERG12 and HIS3^{-32 to -1000}-HISMX-HIS3^{504 to -1103} were generated by PCR amplification as outlined in Table 4. FIG. 2D shows a map of the ERG10-P_{GAL}-ERG12 insert, and SEQ ID NO: 8 shows the nucleotide sequence of the DNA fragment. For construction of pAM495, 400 ng of pAM474 and 100 ng of pAM469 were digested to completion using XmaI restriction enzyme, DNA fragments corresponding to the ERG10-P_{GAL}-ERG12 insert and the linearized pAM469 vector were gel purified, and 4 molar equivalents of the purified insert was ligated with 1 molar equivalent of the purified linearized vector, yielding vector pAM495.

TABLE 4
PCR reactions performed to generate pAM495

| PCR | | | | |
|---|---|---|---|---|
| Round | Template | Primer 1 | Primer 2 | PCR Product |
| 1 | 100 ng of Y007 genomic DNA | 61-67-CPK013-G | 61-67-CPK014alt-G | HIS3^{-32 to -1000} |
| | | (SEQ ID NO: 42) | (SEQ ID NO: 43) | |
| | | 61-67-CPK017-G | 61-67-CPK018-G | HIS3^{504 to -1103} |
| | | (SEQ ID NO: 46) | (SEQ ID NO: 47) | |
| | | 61-67-CPK035-G | 61-67-CPK056-G | ERG10 |
| | | (SEQ ID NO: 57) | (SEQ ID NO: 68) | |
| | | 61-67-CPK57-G | 61-67-CPK058-G | P_{GAL} |
| | | (SEQ ID NO: 69) | (SEQ ID NO: 70) | |
| | | 61-67-CPK040-G | 61-67-CPK059-G | ERG12 |
| | | (SEQ ID NO: 58) | (SEQ ID NO: 71) | |
| | 10 ng of plasmid pAM330 DNA** | 61-67-CPK015alt-G | 61-67-CPK016-G | HISMX |
| | | (SEQ ID NO: 44) | (SEQ ID NO: 45) | |
| 2 | 100 ng each of HIS3^{504 to -1103} and | 61-67-CPK015alt-G | 61-67-CPK018-G | HISMX-HIS3^{504 to -1103} |
| | HISMX PCR purified products | (SEQ ID NO: 44) | (SEQ ID NO: 47) | |
| | 100 ng each of ERG10 and P_{GAL} | 61-67-CPK035-G | 61-67-CPK058-G | ERG10-P_{GAL} |
| | purified PCR products | (SEQ ID NO: 57) | (SEQ ID NO: 70) | |
| 3 | 100 ng each of HIS3^{-32 to -1000} and | 61-67-CPK013-G | 61-67-CPK018-G | HIS3^{-32 to -1000} |
| | HISMX-HIS3^{504 to -1103} purified PCR | (SEQ ID NO: 42) | (SEQ ID NO: 47) | HISMX-HIS3^{504 to -1103} |
| | products | | | |
| | 100 ng each of ERG10-P_{GAL} and | 61-67-CPK035-G | 61-67-CPK040-G | ERG10-P_{GAL}- |
| | ERG12 purified PCR products | (SEQ ID NO: 57) | (SEQ ID NO: 58) | ERG12 |

**The HISMX marker in pAM330 originated from pFA6a-HISMX6-PGAL1 as described by van Dijken et al. ((2000) Enzyme Microb. Technol. 26 (9-10): 706-714).

Plasmid pAM497 was generated by inserting the ERG8-P_{GAL}-ERG19 insert of pAM475 into vector pAM470. Vector pAM475 was generated by inserting DNA fragment ERG8-P_{GAL}-ERG19, which comprises the ORF of the ERGS gene of Saccharomyces cerevisiae (ERG8 nucleotide position 1 to 1512) (ERG8), the genomic locus containing the divergent GAL1 and GAL10 promoter of Saccharomyces cerevisiae (GAL1 nucleotide position −1 to −668) (P_{GAL}), and the ORF of the ERG19 gene of Saccharomyces cerevisiae (ERG19 nucleotide position 1 to 1341) (ERG19), into the TOPO Zero Blunt II cloning vector. Vector pAM470 was generated by inserting DNA fragment LEU2^{-100 to 450}-HISMX-LEU2^{1096 to 1770}, which comprises two segments of the LEU2 locus of Saccharomyces cerevisiae that extend from nucleotide position −100 to position 450 and from nucleotide position 1096 to position 1770, a HISMX marker, and a non-native XmaI restriction site between the LEU2^{1096 to 1770} sequence and the HISMX marker, into the TOPO TA pCR2.1 cloning vector. DNA fragments ERG8-P_{GAL}-ERG19 and LEU2^{-100 to 450}-HISMX-LEU2^{1096 to 1770} were generated by PCR amplification as outlined in Table 5. FIG. 2E for a map of the ERG8-P_{GAL}-ERG19 insert, and SEQ ID NO: 9 shows the nucleotide sequence of the DNA fragment. For the construction of pAM497, 400 ng of pAM475 and 100 ng of pAM470 were digested to completion using XmaI restriction enzyme, DNA fragments corresponding to the ERG8-P_{GAL}-ERG19 insert and the linearized pAM470 vector were purified, and 4 molar equivalents of the purified insert was ligated with 1 molar equivalent of the purified linearized vector, yielding vector pAM497.

TABLE 5
PCR reactions performed to generate pAM497

| PCR | | | | |
|---|---|---|---|---|
| Round | Template | Primer 1 | Primer 2 | PCR Product |
| 1 | 100 ng of Y007 genomic DNA | 61-67-CPK019-G | 61-67-CPK020-G | LEU2^{-100 to 450} |
| | | (SEQ ID NO: 48) | (SEQ ID NO: 49) | |
| | | 61-67-CPK023-G | 61-67-CPK024-G | LEU2^{1096 to 1770} |
| | | (SEQ ID NO: 52) | (SEQ ID NO: 53) | |
| | 10 ng of plasmid pAM330 DNA** | 61-67-CPK021-G | 61-67-CPK022-G | HISMX |
| | | (SEQ ID NO: 50) | (SEQ ID NO: 51) | |
| | 100 ng of Y002 genomic DNA | 61-67-CPK041-G | 61-67-CPK060-G | ERG8 |
| | | (SEQ ID NO: 59) | (SEQ ID NO: 72) | |
| | | 61-67-CPK061-G | 61-67-CPK062-G | P_{GAL} |
| | | (SEQ ID NO: 73) | (SEQ ID NO: 74) | |
| | | 61-67-CPK046-G | 61-67-CPK063-G | ERG19 |
| | | (SEQ ID NO: 60) | (SEQ ID NO: 75) | |
| 2 | 100 ng each of LEU2^{1096 to 1770} and | 61-67-CPK021-G | 61-67-CPK024-G | HISMX-LEU2^{1096 to 1770} |
| | HISMX purified PCR products | (SEQ ID NO: 50) | (SEQ ID NO: 53) | |
| | 100 ng each of ERG8 and P_{GAL} purified | 61-67-CPK041-G | 61-67-CPK062-G | ERG8-P_{GAL} |
| | PCR products | (SEQ ID NO: 59) | (SEQ ID NO: 74) | |
| 3 | 100 ng of LEU2^{-100 to 450} and HISMX- | 61-67-CPK019-G | 61-67-CPK024-G | LEU2^{-100 to 450} |
| | LEU2^{1096 to 1770} purified PCR products | (SEQ ID NO: 31) | (SEQ ID NO: 36) | HISMX-LEU2^{1096 to 1770} |
| | 100 ng each of ERG8-P_{GAL}and ERG19 | 61-67-CPK041-G | 61-67-CPK046-G | ERG8-P_{GAL}-ERG19 |
| | purified PCR products | (SEQ ID NO: 42) | (SEQ ID NO: 43) | |

**The HISMX marker in pAM330 originated from pFA6a-HISMX6-PGAL1 as described by van Dijken et al. ((2000) Enzyme Microb. Technol. 26 (9-10): 706-714).

### Example 2

This example describes methods for making expression plasmids for the introduction of extrachromosomal heterologous nucleic acids comprising galactose-inducible promoters operably linked to protein coding sequences into Saccharomyces cerevisiae.

Expression plasmid pAM353 was generated by inserting a nucleotide sequence encoding a β-farnesene synthase into the pRS425-Gal1 vector (Mumberg et. al. (1994) Nucl. Acids. Res. 22(25): 5767-5768). The nucleotide sequence insert was generated synthetically, using as a template the coding sequence of the β-farnesene synthase gene of Artemisia annua (GenBank accession number AY835398) codon-optimized for expression in Saccharomyces cerevisiae (SEQ ID NO: 10). The synthetically generated nucleotide sequence was flanked by 5′ BamHI and 3′ XhoI restriction sites, and could thus be cloned into compatible restriction sites of a cloning vector such as a standard pUC or pACYC origin vector. The synthetically generated nucleotide sequence was isolated by digesting to completion the DNA synthesis construct using BamHI and XhoI restriction enzymes. The reaction mixture was resolved by gel electrophoresis, the approximately 1.7 kb DNA fragment comprising the β-farnesene synthase coding sequence was gel extracted, and the isolated DNA fragment was ligated into the BamHI XhoI restriction site of the pRS425-Gal1 vector, yielding expression plasmid pAM353.

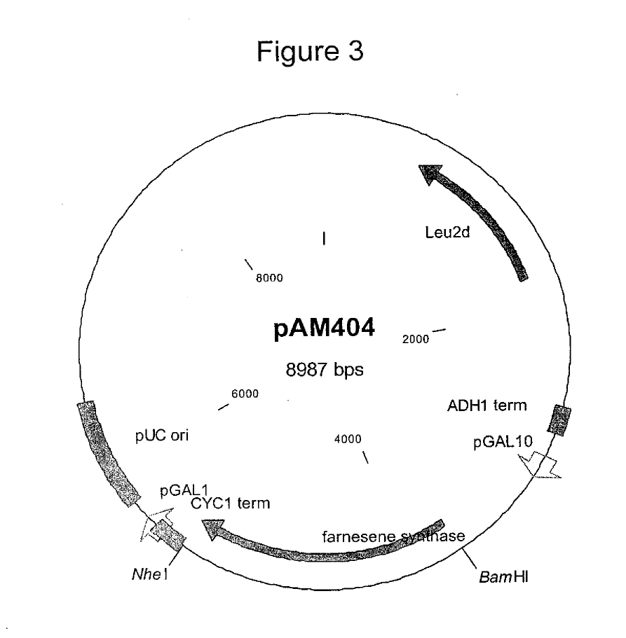

Expression plasmid pAM404 was generated by inserting a nucleotide sequence encoding the β-farnesene synthase of Artemisia annua (GenBank accession number AY835398), codon-optimized for expression in Saccharomyces cerevisiae, into vector pAM178 (SEQ ID NO: 11). The nucleotide sequence encoding the β-farnesene synthase was PCR amplified from pAM353 using primers 52-84 pAM326 BamHI (SEQ ID NO: 108) and 52-84 pAM326 NheI (SEQ ID NO: 109). The resulting PCR product was digested to completion using BamHI and NheI restriction enzymes, the reaction mixture was resolved by gel electrophoresis, the approximately 1.7 kb DNA fragment comprising the β-farnesene synthase coding sequence was gel extracted, and the isolated DNA fragment was ligated into the BamHI NheI restriction site of vector pAM178, yielding expression plasmid pAM404 (see FIG. 3 for a plasmid map).

### Example 3

This example describes methods for making vectors and DNA fragments for the targeted disruption of the gal7/10/1 chromosomal locus of Saccharomyces cerevisiae.

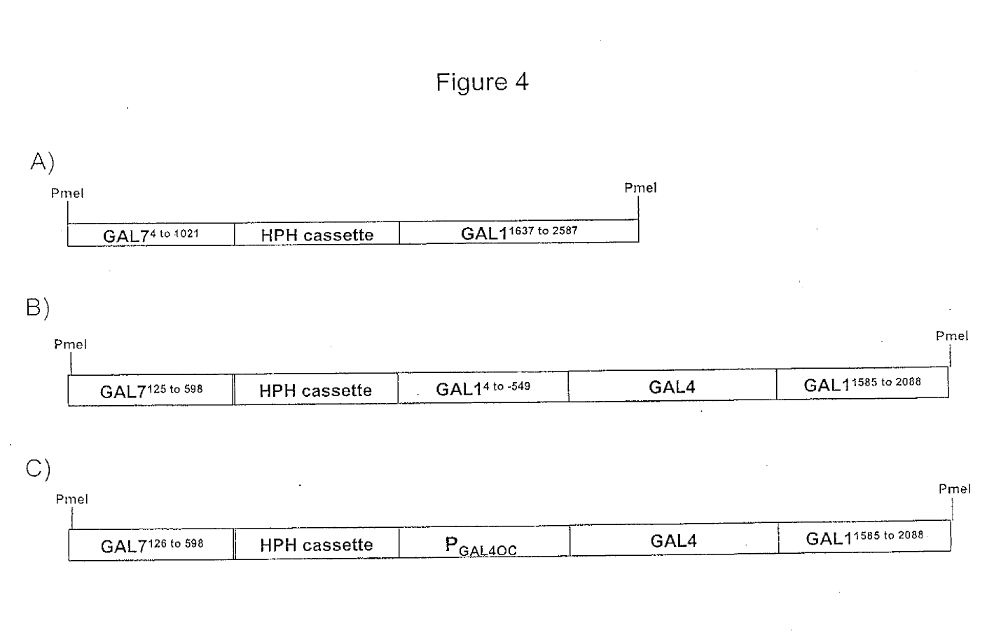

Plasmid pAM584 was generated by inserting DNA fragment GAL7^{4 to 1021}-HPH-GAL1^{1637 to 2587} into the TOPO ZERO Blunt II cloning vector Ivitrogen, Carlsbad, Calif.). DNA fragment GAL7^{4 to 1021}-HPH-GAL1^{1637 to 2587} comprises a segment of the ORF of the GAL7 gene of Saccharomyces cerevisiae (GAL7 nucleotide positions 4 to 1021) (GAL7^{4 to 1021}), the hygromycin resistance cassette (MPH), and a segment of the 3′ untranslated region (U)R of the GAL1 gene of Saccharomyces cerevisiae (GAL1 nucleotide positions 1637 to 2587). The DNA fragment was generated by PCR amplification as outlined in Table 6. FIG. 4A shows a map and SEQ ID NO: 12 the nucleotide sequence of DNA fragment GAL7^{4 to 1021}-HPH-GAL1^{637 to 2587}.

TABLE 6
PCR reactions performed to generate pAM584

| PCR | | | | |
|---|---|---|---|---|
| Round | Template | Primer 1 | Primer 2 | PCR Product |
| 1 | 100 ng of Y002 genomic DNA | 91-014-CPK236-G | 91-014-CPK237-G | GAL7^{4 to 1021} |
| | | (SEQ ID NO: 83) | (SEQ ID NO: 84) | |
| | | 91-014-CPK232-G | 91-014-CPK233-G | GAL1^{1637 to 2587} |
| | | (SEQ ID NO: 81) | (SEQ ID NO: 82) | |
| | 10 ng of plasmid pAM547 DNA** | 91-014-CPK231-G | 91-014-CPK238-G | HPH |
| | | (SEQ ID NO: 80) | (SEQ ID NO: 85) | |
| 2 | 100 ng each of GAL7^{4 to 1021} and HPH | 91-014-CPK231-G | 91-014-CPK236-G | GAL7^{4 to 1021}-HPH |
| | purified PCR products | (SEQ ID NO: 80) | (SEQ ID NO: 83) | |
| 3 | 100 ng of each GAL1^{1637 to 2587} and | 91-014-CPK233-G | 91-014-CPK236-G | GAL7^{4 to 1021}-HPH- |
| | GAL7^{4 to 1021}-HPH purified PCR | (SEQ ID NO: 82) | (SEQ ID NO: 83) | GAL1^{1637 to 2587} |
| | products | | | |

**Plasmid pAM547 was generated synthetically, and comprises the HPH cassette, which consists of the coding sequence for the hygromycin B phosphotransferase of Escherichia coli flanked by the promoter and terminator of the Tef1 gene of Kluyveromyces lactis.

Plasmid pAM610 was generated by inserting DNA fragment GAL7125 to 598-PH-GAL1^{4 to -549}-GAL4-GAL1^{1585 to 2088} into the TOPO ZERO Blunt TI cloning vector (Invitrogen, Carlsbad, Calif.). DNA fragment GAL7^{125 to 598}-HPH-GAL1^{4 to -549} GAL4-GAL1^{1585 to 2058} comprises a segment of the ORF of the GAL7 gene of Saccharomyces cerevisiae (GAL7 nucleotide positions 125 to 598) (GAL7125 to 598), the hygromycin resistance cassette (HPH), a segment of the 5′ UTR of the GAL1 gene of Saccharomyces cerevisiae (GAL1 nucleotide positions 4 to −549) (GAL1^{4 to -}549), the ORF of the GAL4 gene of Saccharomyces cerevisiae (GAL4), and a segment of the 3′ UTR of the GAL1 gene of Saccharomyces cerevisiae (GAL1^{1585 to 2088}). The DNA fragment was generated by PCR amplification as outlined in Table 7. FIG. 4B shows a map and SEQ ID NO: 13 the nucleotide sequence of DNA fragment GAL7^{125 to 598}-HPH-GAL1^{4 to 549}-GAL4-GAL1^{1585 to 2088}.

TABLE 7
PCR amplifications performed to generate pAM610

| PCR | | | | |
|---|---|---|---|---|
| Round | Template | Primer 1 | Primer 2 | PCR Product |
| 1 | 100 ng of Y002 genomic DNA | 91-035-CPK277-G | 91-035-CPK278-G | GAL7^{125 to 598} |
| | | (SEQ ID NO: 86) | (SEQ ID NO: 87) | |
| | | 91-093-CPK285 | 91-093-CPK286 | GAL1^{1585 to 2088} |
| | | (SEQ ID NO: 104) | (SEQ ID NO: 105) | |
| | | 91-035-CPK281-G | 91-035-CPK282-G | GAL1^{4 to -549} |
| | | (SEQ ID NO: 90) | (SEQ ID NO: 91) | |
| | | 91-035-CPK283-G | 91-035-CPK284-G | GAL4 |
| | | (SEQ ID NO: 92) | (SEQ ID NO: 93) | |
| | 10 ng of pAM547 plasmid DNA** | 91-035-CPK279-G | 91-035-CPK280-G | HPH |
| | | (SEQ ID NO: 88) | (SEQ ID NO: 89) | |
| 2 | 50 ng each of the purified GAL7^{125 to 598}, | 91-035-CPK277-G | 91-093-CPK286 | GAL7^{125 to 598}-HPH- |
| | HPH, GAL1^{4 to -549}, GAL4, and | (SEQ ID NO: 86) | (SEQ ID NO: 105) | GAL1^{4 to -549}-GAL4- |
| | GAL1^{1585 to 2088} purified PCR products | | | GAL1^{1585 to 2088} |

**Plasmid pAM547 was generated synthetically, and comprises the HPH cassette, which consists of the coding sequence for the hygromycin B phosphotransferase of Escherichia coli flanked by the promoter and terminator of the Tef1 gene of Kluyveromyces lactis.

DNA fragment GAL7^{126 to 598}-HPH-P_{GAL4OC}-GAL4-GAL1^{1585 to 2088}, which comprises a segment of the ORE of the GAL7 gene of Saccharomyces cerevisiae (GAL7 nucleotide positions 126 to 598) (GAL7^{126 to 598}), the hygromycin resistance cassette (HPH), the ORF of the GAL4 gene of Saccharomyces cerevisiae under the control of an “coperative constitutive” version of its native promoter (Griggs & Johnston (1991) PNAS 88(19):8597-8601) (P_{Gal4OC}-GAL4), and a segment of the 3′ UTR of the Gal1 gene of Saccharomyces cerevisiae (GAL1 nucleotide positions 1585 to 2088) (GAL1^{1585 to 2088}), was generated by PCR amplification as outlined in Table 8. FIG. 4C shows a map and SEQ ID NO: 14 the nucleotide sequence of DNA fragment GAL7^{126 to 598}-HPH-P_{GAL4OC}-GAL4-GAL1^{1585 to 2088}.

TABLE 8
PCR amplifications performed to generate DNA fragment GAL7^{126 to 598}-HPH-P_{GAL4OC}-GAL4-GAL1^{1585 to 2088}

| PCR | | | | |
|---|---|---|---|---|
| Round | Template | Primer 1 | Primer 2 | PCR Product |
| 1 | 100 ng of pAM610 plasmid DNA | 91-093-CPK285 | 91-093-CPK286 | GAL1^{1585 to 2088} |
| | | (SEQ ID NO: 104) | (SEQ ID NO: 105) | |
| | | 91-093-CPK277 | 91-093-CPK421-G | GAL7^{126 to 598}-HPH |
| | | (SEQ ID NO: 102) | (SEQ ID NO: 106) | |
| | 100 ng of pAM629 plasmid DNA** | 91-093-CPK422-G | 91-093-CPK284-G | P_{GAL4OC}-GAL4 |
| | | (SEQ ID NO: 107) | (SEQ ID NO: 103) | |
| 2 | 50 ng of GAL1^{1585 to 2088}, 200 ng of | 91-093-CPK277 | 91-093-CPK286 | GAL7^{126 to 598}-HPH- |
| | GAL7^{126 to 598}-HPH, and 241 ng of | (SEQ ID NO: 102) | (SEQ ID NO: 105) | P_{GAL4OC}-GAL4- |
| | P_{GAL4OC}-GAL4 purified PCR product | | | GAL1^{1585 to 2088} |

**The insert of plasmid pAM629 was stitched together from DNA fragments that were PCR amplified from Y002 genomic DNA using primer pairs 100-30-KB011-G (SEQ ID NO: 18) and 100-30-KB012-G (SEQ ID NO: 19), and 100-30-KB013-G (SEQ ID NO: 20) and 100-30-KB014-G (SEQ ID NO: 21).

### Example 4

This example describes methods for making DNA fragments for the targeted integration into specific chromosomal locations of Saccharomyces cerevisiae of nucleic acids encoding lactases and lactose transporters.

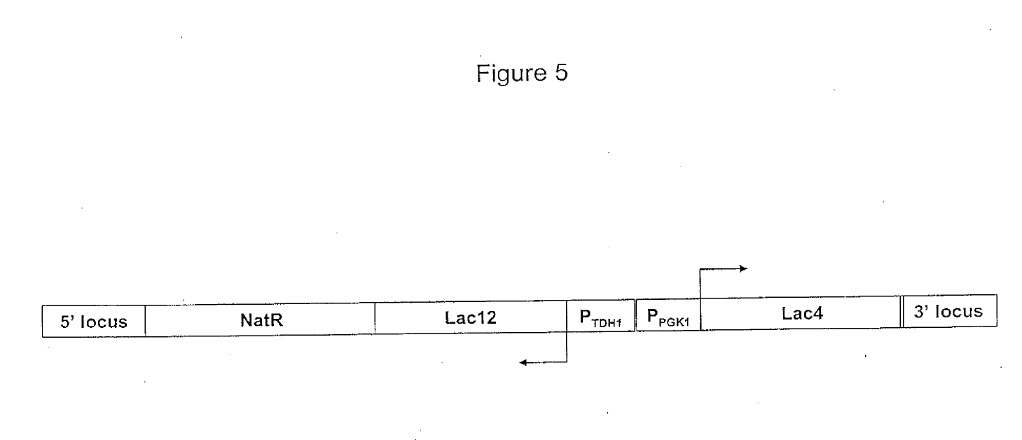

DNA fragment 5′ locus-NatR-LAC12-P_{TDH1}-P_{PGK1}-LAC4-3′ locus, which comprises a segment of the 5′ UTR of the ERG9 gene (3′ locus), the nourseothricin resistance selectable marker gene of Streptomyces noursei NatR), the ORF of the LAC12 gene of Kluyveromyces lactis (X06997 REGION: 1616 . . . 3379) (LAC 12) operably linked to the promoter of the TDH1 gene of Saccharomyces cerevisiae (P_{TDH1}), the ORF of the LAC4 gene of Kluyveromyces lactis (M84410 REGION: 43 . . . 3382) (LAC4) operably linked to the promoter of the PGK1 promoter of Saccharomyces cerevisiae (P_{PGK1}), and the MET3 promoter region (5′ locus) of plasmid pAM625, is generated by PCR amplification as outlined in Table 9. FIG. 5 shows a map and SEQ ID NO: 15 the nucleotide sequence of DNA fragment 5′ locus-NatR-LAC12-P_{TDH1}-P_{PGK1}-LAC₄-3′ locus.

TABLE 9
PCR amplifications performed to generate DNA fragment 5′ locus-NatR-LAC12-P_{TDH1}-P_{PGK1}-LAC4-3′ locus

| PCR | | | | |
|---|---|---|---|---|
| Round | Template | Primer 1 | Primer 2 | PCR Product |
| 1 | 6.25 ng of Kluyveromyces lactis | LAC4-1 | LAC4-2 | LAC4 |
| | genomic DNA (ATCC catalog# 8585D- | (SEQ ID NO: 112) | (SEQ ID NO: 113) | |
| | 5, Lot# 7495280) | LAC12-1 | LAC12-2 | LAC12 |
| | | (SEQ ID NO: 110) | (SEQ ID NO: 111) | |
| | 6.25 ng of Y002 genomic DNA | P_{PGK1}-1 | P_{PGK1}-2 | P_{PGK1} |
| | | (SEQ ID NO: 116) | (SEQ ID NO: 117) | |
| | | P_{TDH1}-1 | P_{TDH1}-2 | P_{TDH1} |
| | | (SEQ ID NO: 22) | (SEQ ID NO: 23) | |
| | 400 ug of pAM625 plasmid DNA^{a)} | 5′ locus-1 | 5′ locus-2 | 5′ locus |
| | | (SEQ ID NO: 26) | (SEQ ID NO: 27) | |
| | | 3′ locus-1 | 3′ locus-2 | 3′ locus |
| | | (SEQ ID NO: 24) | (SEQ ID NO: 25) | |
| | 400 ug of pAM700 plasmid DNA^{b)} | NatR-1 (SEQ ID NO: | NatR-2 (SEQ ID NO: | NatR |
| | | 114) | 115) | |
| 2 | 0.15 pM of each of LAC4, LAC12, | 5′ locus-1 (SEQ ID | 3′ locus-2(SEQ ID | 5′ locus-NatR- |
| | P_{PGK1}, P_{TDH1}, 5′ locus, 3′ locus, and | NO: 26) | NO: 25) | LAC12-P_{TDH1}- |
| | NatR purified PCR products | | | P_{PGK1}-LAC4-3′ |
| | | | | locus |

^{a)}Plasmid pAM625 was generated by inserting DNA fragment ERG9^{-1 to -800}-DsdA-P_{MET3}^{-1 to -683}-ERG9^{1 to 811} (see Example 5) into the TOPO ZERO Blunt II cloning vector.
^{b)}Plasmid pAM700 comprises a nucleotide sequence that encodes the nourseothricin acetyltransferase of Streptomyces noursei (GenBank accession X73149 REGION: 179 . . . 748) flanked by the promoter and terminator of the Tef1 gene of Kluyveromyces lactis.

### Example 5

This example describes the generation of Saccharomyces cerevisiae strains useful in the invention.

Saccharomyces cerevisiae strains CEN.PK2-1C (Y002) (MATA; ura3-52; tup1-289; leu2-3, 112; his3661; MAL2-8C; SUC2) and CEN.PK2-1D (Y003) (MATalpha; ura3-52; trp1-289; leu2-3, 112; his3Δ1; MAL2-8C; SUC2) (van Dijken et al (2000) Enzyme Microb. Technol 26(9-10):706-714) were prepared for introduction of inducible MEV pathway genes by replacing the ERG9 promoter with the Saccharomyces cerevisiae MET3 promoter, and the ADE1 ORE with the Candida glabrata LEU2 gene (CgLEU2). This was done by PCR amplifying the KanMX-P_{MET3} region of vector pAM328 (SEQ ID NO: 16) using primers 50-56-pw100-G (SEQ ID NO: 28) and 50-56-pw101-G (SEQ ID NO: 29), which include 45 base pairs of homology to the native ERG9 promoter, transforming 10 ug of the resulting PCR product into exponentially growing Y002 and Y003 cells using 40% w/w Polyethelene Glycol 3350 (Sigma-Aldrich, St. Louis, Mo.), 100 mM Lithium Acetate (Sigma-Aldrich, St. Louis, Mo.), and 10 ug Salmon Sperm DNA (Invitrogen Corp., Carlsbad, Calif.), and incubating the cells at 30° C. for 30 minutes followed by heat shocking them at 42° C. for 30 minutes (Schiestl and Gietz. (1989) Curr. Genet. 16, 339-346). Positive recombinants were identified by their ability to grow on rich medium containing 0.5 ug/ml Geneticin (Tavitrogen Corp., Carlsbad, Calif.), and selected colonies were confirmed by diagnostic PCR. The resultant clones were given the designation Y93 WAT A) and Y94 (MAT alpha). The 3.5 kb CgLEU2 genomic locus was then amplified from Candida glabrata genomic DNA (ATCC, Manassas, Va.) using primers 61-67-CPK066-G (SEQ ID NO: 78) and 61-67-CPK067-G (SEQ ID NO: 79), which contain 50 base pairs of flanking homology to the ADE1 ORF, and 10 ug of the resulting PCR product were transformed into exponentially growing Y93 and Y94 cells, positive recombinants were selected for growth in the absence of leucine supplementation, and selected clones were confirmed by diagnostic PCR. The resultant clones were given the designation Y176 (MAT A) and Y177 (MAT alpha).

Strain Y188 was then generated by digesting 2 ug of pAM491 and pAM495 plasmid DNA to completion using PmeI restriction enzyme (New England Biolabs, Beverly, Mass.), and introducing the purified DNA inserts into exponentially growing Y176 cells. Positive recombinants were selected for by growth on medium lacking uracil and histidine, and integration into the correct genomic locus was confirmed by diagnostic PCR.

Strain Y189 was next generated by digesting 2 ug of pAM489 and pAM497 plasmid DNA to completion using Pmelrestriction enzyme, and introducing the purified DNA inserts into exponentially growing Y177 cells. Positive recombinants were selected for by growth on medium lacking tryptophan and histidine, and integration into the correct genomic locus was confirmed by diagnostic PCR.

Approximately 1×10⁷ cells from strains Y188 and Y189 were mixed on a YPD medium plate for 6 hours at room temperature to allow for mating. The mixed cell culture was plated to medium lacking histidine, uracil, and trptophan to select for growth of diploid cells. Strain Y238 was generated by transforming the diploid cells using 2 ug of pAM493 plasmid DNA that had been digested to completion using Pmel restriction enzyme, and introducing the purified DNA insert into the exponentially growing diploid cells. Positive recombinants were selected for by growth on medium lacking adenine, and integration into the correct genomic locus was confirmed by diagnostic PCR.

Haploid strain Y211 (MAT alpha) was generated by sporulating strain Y238 in 2% Potassium Acetate and 0.02% Raffinose liquid medium, isolating approximately 200 genetic tetrads using a Singer Instruments MSM300 series micromanipulator (Singer Instrument LTD, Somerset, UK), identifying independent genetic isolates containing the appropriate complement of introduced genetic material by their ability to grow in the absence of adenine, histidine, uracil, and tryptophan, and confirming the integration of all introduced DNA by diagnostic PCR.

Strain Y381 was generated from strain Y211 by removing 69 nucleotides of the native ERG9 locus between the engineered MET3 promoter and start of the ERG9 coding sequence, thus rendering expression of ERG9 more methionine repressible, and by replacing the Kar marker at this site with another selectable marker. To this end, exponentially growing Y211 cells were transformed with 100 ug of DNA fragment ERG9^{-1 to -800}-DsdA-P_{MET3}-ERG9^{1 to 811} DNA fragment ERG9^{-1 to -800}-DsdA-P_{MET3}-ERG9^{1 to 811} (SEQ ID NO: 17) comprises a segment of the 5′ UTR of the ERG9 gene of Saccharomyces cerevisiae (ERG9 nucleotide positions −1 to −800) (ERG9^{-1 to -800}), the DsdA selectable marker (DsdA), the promoter region of the MET3 gene of Saccharomyces cerevisiae (MET3 nucleotide positions −2 to −687) (P_{MET3}), and a segment of the ORF of the ERG9 gene (ERG9 nucleotide positions 1 to 811) (ERG9^{1 to 811}). The DNA fragment was generated by PCR amplification as outlined in Table 10. Host cell transformants were selected on synthetic defined media containing 2% glucose and D-serine, and integration into the correct genomic locus was confirmed by diagnostic PCR.

TABLE 10
PCR amplifications performed to generate DNA fragment ERG9^{-1 to -800}-DsdA-P_{MET3}-ERG9^{1 to 811}

| PCR | | | | |
|---|---|---|---|---|
| Round | Template | Primer 1 | Primer 2 | PCR Product |
| 1 | 100 ng of Y002 genomic DNA | 91-044-CPK320-G | 91-044-CPK321-G | ERG9^{-1 to -800} |
| | | (SEQ ID NO: 94) | (SEQ ID NO: 95) | |
| | | 91-044-CPK324-G | 91-044-CPK325-G | P_{MET3} |
| | | (SEQ ID NO: 98) | (SEQ ID NO: 99) | |
| | | 91-044-CPK326-G | 91-044-CPK327-G | ERG9^{1 to 811} |
| | | (SEQ ID NO: 100) | (SEQ ID NO: 101) | |
| | 10 ng of pAM577 plasmid DNA** | 91-044-CPK322-G | 91-044-CPK323-G | DsdA |
| | | (SEQ ID NO: 96) | (SEQ ID NO: 97) | |
| 2 | 100 ng each of ERG9^{-1 to -800}, DsdA, | 91-044-CPK320-G | 91-044-CPK327-G | ERG9^{-1 to -800}-DsdA- |
| | P_{MET3}, and ERG9^{1 to 811} purified PCR | (SEQ ID NO: 94) | (SEQ ID NO: 101) | P_{MET3}-ERG9^{1 to 811} |
| | products | | | |

**Plasmid pAM577 was generated synthetically, and comprises a nucleotide sequence that encodes the D-serine deaminase of Saccharomyces cerevisiae.

Strain Y435 was generated from strain Y381 by rendering the strain unable to catabolize galactose, able to express higher levels of GAL4p in the presence of glucose (i.e., able to more efficiently drive expression off galactose-inducible promoters in the presence of glucose, as well as assure that there is enough Gal4p transcription factor to drive expression from all the galactose-inducible promoters in the cell), and able to produce β-farnesene synthase in the presence of galactose. To this end, exponentially growing Y381 cells were first transformed with 850 ng of gel purified DNA fragment GAL7^{126 to 598}-HPH-P_{GAL4OC}-GAL4-GAL1^{1585 to 2088}. Host cell transformants were selected on YPD agar containing 200 ug/mL hygromycin B, single colonies were picked, and integration into the correct genomic locus was confirmed by diagnostic PCR. Positive colonies were re-streaked on YPD agar containing 200 ug/uL hygromycin B to obtain single colonies for stock preparation. One such positive transforannt strain was then transformed with expression plasmid pAM404, yielding strain Y435. Host cell transformants were selected on synthetic defined media, containing 2% glucose and all amino acids except leucine and methionine (SM-leu-met). Single colonies were transferred to culture vials containing 5 mL of liquid SM-leu-met, and the cultures were incubated by shaking at 30° C. until growth reached stationary phase. The cells were stored at −80° C. in cryo-vials in 1 mL frozen aliquots made up of 400 uL 50% sterile glycerol and 600 uL liquid culture.

Strain Y596 was generated from strain Y435 by rendering the strain capable of producing a lactase and a lactose transporter. To this end, exponentially growing Y435 cells were transformed with 4 ug of gel purified DNA fragment 5′ locus-NatR-LAC12-P_{TDH1}-P_{PGK1}-LAC4-3′ locus. Positive recombinants were selected for by growth on YPD medium comprising 200 ug nourseothricin, and integration into the correct genomic locus was confirmed by diagnostic PCR. Single colonies were transferred to culture vials containing 5 mL of liquid YPD, and the cultures were incubated by shaking at 30° C. until growth reached stationary phase. The cells were stored at −80° C. in cryo-vials in 1 mL frozen aliquots made up of 400 uL 50% sterile glycerol and 600 uL liquid culture.

### Example 6

This example describes the production of β-farnesene in Saccharomyces cerevisiae host strains grown in the presence of lactose.

Seed cultures of host strains Y435 and Y596 were established by adding stock aliquots to a 125 mL flask containing 25 mL Bird's Production media, and growing the cultures overnight. Each seed culture was used to inoculate at an initial OD₆₀₀ of approximately 0.05 each of two 20 mL baffled flasks containing 40 mL of Bird's Production media containing 2% glucose and either 5.0 g/L galactose, or 9.6 g/L, 6.0 g/L, or 2.4 g/L lactose. The cultures were overlain with 8 mL methyl oleate, and incubated at 30° C. on a rotary shaker at 200 rpm. Triplicate samples were taken every 24 hours up to 72 hours by transferring 2 uL to 10 uL of the organic overlay to a clean glass vial containing 500 uL ethyl acetate spiked with beta- or trans-caryophyllene as an internal standard.

The ethyl acetate samples were analyzed on an Agilent 6890N gas chromatograph equipped with a flame ionization detector (Agilent Technologies Inc., Palo Alto, Calif.). Compounds in a 1 μL aliquot of each sample were separated using a DB-1MS column (Agilent Technologies, Inc., Palo Alto, Calif.), helium carrier gas, and the following temperature program: 200° C. hold for 1 minute, increasing temperature at 10° C./minute to a temperature of 230° C., increasing temperature at 40° C./minute to a temperature of 300° C., and a hold at 300° C. for 1 minute. Using this protocol, β-farnesene had previously been shown to have a retention time of approximately 2 minutes. Farnesene titers were calculated by comparing generated peak areas against a quantitative calibration curve of purified O-farnesene (Sigma-Aldrich Chemical Company, St. Louis, Mo.) in trans-caryophyllene-spiked ethyl acetate.

Lactose was analyzed on an Agilent 1200 high performance liquid chromatograph using a refractive index detector (Agilent Technologies Inc., Palo Alto, Calif.). Samples were prepared by taking a 500 μL aliquot of clarified fermentation broth and diluting it with an equal volume of 30 mM sulfuric acid. Compounds in a 10 μL aliquot of each sample were separated using a Waters IC-Pak column with 15 mM sulfuric acid as the mobile phase at a flow rate of 0.6 mL/min. Lactose levels were measured by comparing generated peak areas against a quantitative calibration curve of authentic compound.

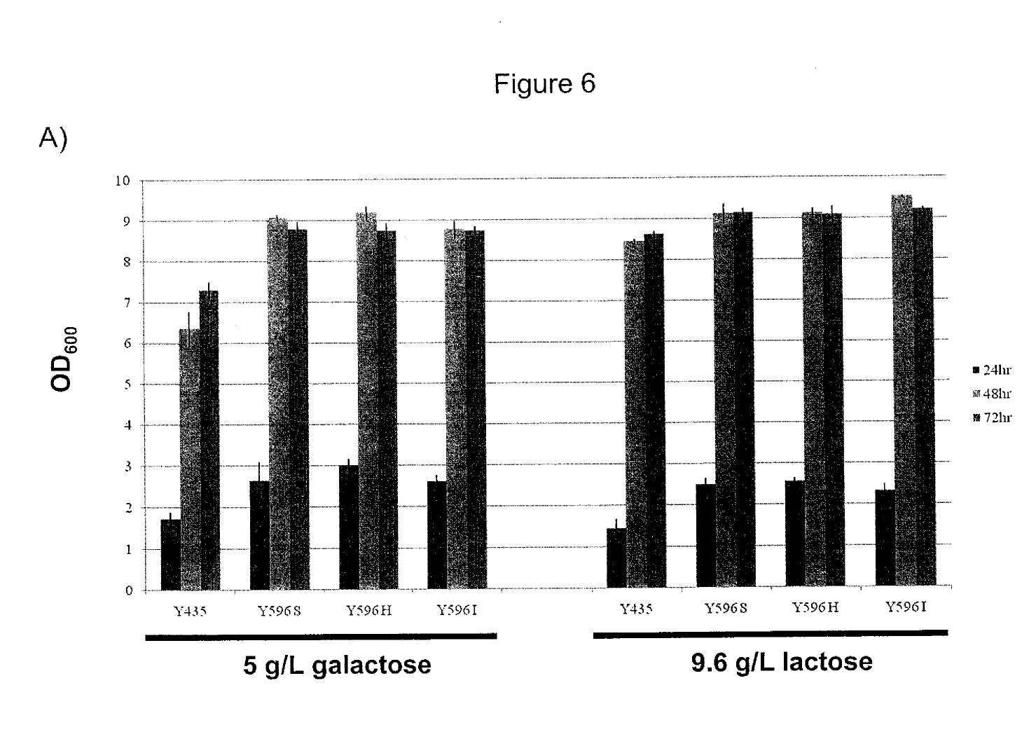

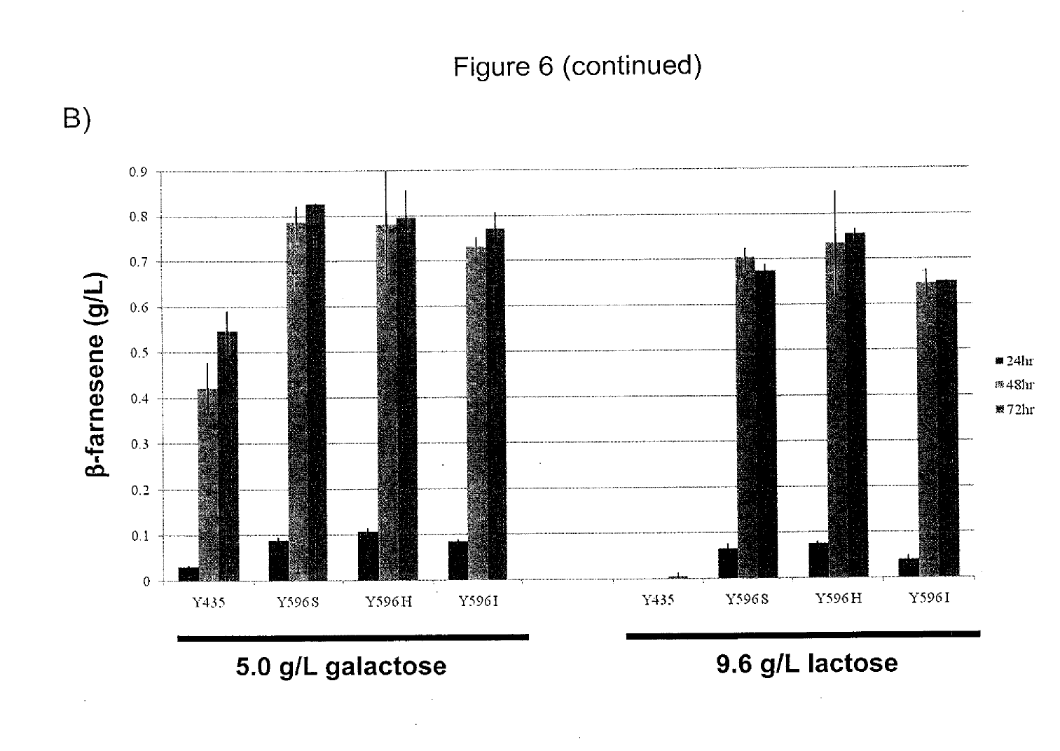

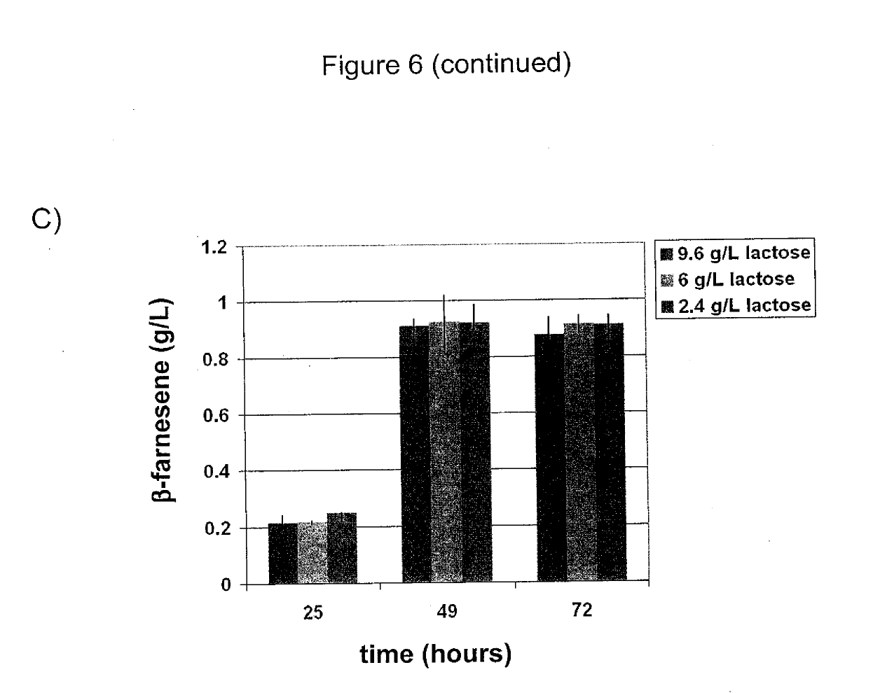

As shown in FIG. 6A, culture growth was similar for each of the two strains regardless of whether the culture medium contained galactose or lactose. As shown in FIG. 6B, strain Y596 produced more than 0.6 g/L β-farnesene both in the presence of galactose and in the presence of lactose whereas control strain Y435 produced β-farnesene only in the presence of inducer galactose but not in the presence of lactose. As shown in FIG. 6C, no more than 2.4 g/L lactose was needed to induce production of β-farnesene by strain Y596.

While the invention has been described with respect to a limited number of embodiments, the specific features of one embodiment should not be attributed to other embodiments of the invention. No single embodiment is representative of all aspects of the claimed subject matter. In some embodiments, the compositions or methods may include numerous compounds or steps not mentioned herein. In other embodiments, the compositions or methods do not include, or are substantially free of, any compounds or steps not enumerated herein. Variations and modifications from the described embodiments exist. It should be noted that the application of the jet fuel compositions disclosed herein is not limited to jet engines; they can be used in any equipment which requires a jet fuel. Although there are specifications for most jet fuels, not all jet fuel compositions disclosed herein need to meet all requirements in the specifications. It is noted that the methods for making and using the jet fuel compositions disclosed herein are described with reference to a number of steps. These steps can be practiced in any sequence. One or more steps may be omitted or combined but still achieve substantially the same results. The appended claims intend to cover all such variations and modifications as falling within the scope of the invention.

All publications and patent applications mentioned in this specification are herein incorporated by reference to the same extent as if each individual publication or patent application was specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. (canceled)

2. (canceled)

3. A method of expressing a heterologous sequence in a host cell, comprising: culturing said host cell in a medium and under conditions such that said heterologous sequence is expressed, wherein said heterologous sequence is operably linked to a galactose-inducible regulatory element, and expression of said heterologous sequence is induced upon addition of lactose to said medium.

4. The method of claim 3, wherein expression of said heterologous sequence is induced upon supplementing lactose and to a level comparable to that obtained by culturing said host cell in a galactose-supplemented medium, wherein quantities of the supplemented galactose and lactose are comparable as measured in moles.

5. The method of claim 3, wherein said heterologous sequence encodes a proteinaceous product.

6. The method of claim 3, wherein said heterologous sequence produces a product selected from the group consisting of: antisense molecules, siRNA, miRNA, EGS, aptamers, and ribozymes.

7. The method of claim 3 wherein the method produces an isoprenoid in a host cell and the host cell expresses one or more heterologous sequences encoding one or more enzymes in a mevalonate-independent deoxyxylulose 5-phosphate (DXP) pathway or mevalonate (MEV) pathway.

8. The method of claim 7, the expression of said one or more heterologous sequences is induced in the presence of lactose.

9. The method of claim 7, wherein said isoprenoid is a C₅-C₂₀ isoprenoid.

10. The method of claim 7, wherein said isoprenoid is a C₂₀₊ isoprenoid.

11. The method of claim 7, wherein said host cell further comprises an exogenous sequence encoding a prenyltransferase and an isoprenoid synthase.

12. The method of claim 7, wherein said medium comprises lactose and lactase.

13. The method of claim 7, wherein said host cell comprises a galactose transporter or biologically active fragment thereof.

14. The method of claim 7, wherein said host cell comprises GAL2 galactose transporter or biologically active fragment thereof.

15. The method of claim 7, wherein said host cell comprises a lactose transporter or biologically active fragment thereof.

16. The method of claim 7, wherein said host cell comprises a galactose transporter that is GAL2.

17. The method of claim 7, wherein said galactose-inducible regulatory element is episomal.

18. The method of claim 7, wherein said galactose-inducible regulatory element is integrated into the genome of said host cell.

19. The method of claim 7, wherein said galactose-inducible regulatory element comprises a galactose-inducible promoter selected from the group consisting of a GAL7, GAL2, GAL1, GAL10, GAL3, GCY1, and GAL80 promoter.

20. The method of claim 7, wherein said host cell comprises a lactase or biologically active fragment thereof.

21. The method of claim 7, wherein said host cell comprises an exogenous sequence encoding a lactase enzyme.

22. The method of claim 7, wherein said host cell comprises an exogenous sequence encoding a secretable lactase.

23. The method of claim 7, wherein said host cell exhibits a reduced capability to catabolize galactose.

24. The method of claim 7, wherein said host cell lacks a functional GAL1, GAL7, and/or GAL10 protein.

25. The method of claim 7, wherein said host cell expresses GAL4 protein.

26. The method of claim 25, wherein said host cell expresses GAL4 protein under the control of a constitutive promoter.

27. The method of claim 7, wherein said host cell is a prokaryotic cell.

28. The method of claim 7, wherein said host cell is a eukaryotic cell.

29. The method of claim 7, wherein said host cell is a fungal cell.

30. A host cell for expressing a heterologous sequence of claim 3.

31. The host cell of claim 30, wherein expression of said heterologous sequence is induced by a non-galactose sugar and to a level comparable to that obtained by culturing said host cell in a galactose-supplemented medium, wherein quantities of the supplemented galactose and non-galactose sugar are comparable as measured in moles.

32. A host cell of claim 30, wherein the heterologous sequence is operably linked to a galactose-inducible regulatory element, and wherein expression of said heterologous sequence is induced in the presence of lactose.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. The host cell of claim 30 or 32 that produces an isoprenoid via deoxyxylulose 5-phosphate (DXP) pathway, wherein the heterologous sequence encodes one or more enzymes in mevalonate-independent deoxyxylulose 5-phosphate (DXP) pathway.

53. The host cell of claim 30 or 32 that produces an isoprenoid via mevalonate (MEV) pathway, wherein the heterologous sequence encodes one or more enzymes in the MEV pathway.

54. The host cell of claim 53, wherein said isoprenoid is a C₅-C₂₀ isoprenoid.

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. A cell culture comprising a host cell of claim 30 or 32.

72. The method of claim 7, wherein the isoprenoid is sesquiterpene.

73. The host cell of claim 52, wherein the isoprenoid is sesquiterpene.
